SEQUENCE LISTING (51 sequences)

SEQ ID NO: 1 - RNA, 2887 nt, synthetic construct
  source 1..2887: mol_type = other RNA
taatacgactcactatagggccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggaagatgccaagaacatcaagaagggccctgctccattctaccctctggaagatggaacagccggcgagcagctgcacaaggccatgaagagatatgccctggtgcctggcacaatcgccttcacagatgcccacatcgaggtggacatcacctacgccgagtacttcgagatgtctgtgcggctggccgaagctatgaagcgctacggcctgaacaccaaccaccggatcgtcgtgtgcagcgagaacagcctgcagttcttcatgcctgtgctgggcgccctgttcatcggagttgctgtggcccctgccaacgacatctacaacgagcgcgagctgctgaacagcatgggcatctctcagcccaccgtggtgttcgtgtccaagaagggactgcagaaaatcctgaacgtgcagaagaagctgcccatcatccagaaaatcatcatcatggacagcaagaccgactaccagggcttccagagcatgtacaccttcgtgaccagccatctgccacctggcttcaacgagtacgacttcgtgcccgagagcttcgaccgggacaagacaatcgccctgatcatgaacagcagcggctctaccggactgcccaaaggtgttgccctgcctcatagaactgcctgcgtgcggtttagccacgccagagatcccatcttcggcaaccagatcatccccgacacagccatcctgagcgtggtgccttttcaccacggcttcggcatgttcaccacactgggctacctgatctgcggcttcagagtggtgctgatgtaccgcttcgaggaagaactgttcctgcggagcctgcaggactacaagatccagtctgctctgctggtgcctactctgttcagcttctttgccaagagcaccctgatcgataagtacgacctgagcaacctgcacgagatcgcctctggcggagcccctctgtctaaagaagtgggagaagccgtcgccaagcggttccatctgcctggcatcagacaaggctatggcctgaccgagacaaccagcgccattctgattacccctgagggcgacgataagcctggcgctgtgggaaaagtggtgccattcttcgaggccaaggtggtggatctggacaccggcaaaacactgggcgttaaccagaggggcgagctgtgtgttagaggccctatgatcatgagcggctacgtgaacaaccccgaggccacaaacgctctgatcgacaaggatggatggctgcacagcggcgacattgcctactgggacgaagatgagcacttcttcatcgtggaccggctgaagtccctgatcaagtacaagggctaccaggtggccccagccgagctggaatctattctgctgcaacaccccaacatcttcgatgccggcgttgcaggactgcccgatgatgatgctggcgaactgccagctgctgtggtggtgctggaacacggcaagaccatgaccgagaaagaaatcgtggactacgtggccagccaagtgaccaccgccaagaaactgagaggcggcgtggtgtttgtggacgaggtgccaaaaggcctgacaggcaagctggacgcccggaagatcagagagatcctgattaaggccaagaaaggcggcaagatcgccgtgtgaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcata

SEQ ID NO: 2 - RNA, 2711 nt, synthetic construct
  source 1..2711: mol_type = other RNA
taatacgactcactatagggccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgggccctatgatcatgagcggctacgtgaacaaccccgaggccacaaacgctctgatcgacaaggatggatggctgcacagcggcgacattgcctactgggacgaagatgagcacttcttcatcgtggaccggctgaagtccctgatcaagtacaagggctaccaggtggccccagccgagctggaatctattctgctgcaacaccccaacatcttcgatgccggcgttgcaggactgcccgatgatgatgctggcgaactgccagctgctgtggtggtgctggaacacggcaagaccatgaccgagaaagaaatcgtggactacgtggccagccaagtgaccaccgccaagaaactgagaggcggcgtggtgtttgtggacgaggtgccaaaaggcctgacaggcaagctggacgcccggaagatcagagagatcctgattaaggccaagaaaggcggcaagatcgccgtgtgattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggaagatgccaagaacatcaagaagggccctgctccattctaccctctggaagatggaacagccggcgagcagctgcacaaggccatgaagagatatgccctggtgcctggcacaatcgccttcacagatgcccacatcgaggtggacatcacctacgccgagtacttcgagatgtctgtgcggctggccgaagctatgaagcgctacggcctgaacaccaaccaccggatcgtcgtgtgcagcgagaacagcctgcagttcttcatgcctgtgctgggcgccctgttcatcggagttgctgtggcccctgccaacgacatctacaacgagcgcgagctgctgaacagcatgggcatctctcagcccaccgtggtgttcgtgtccaagaagggactgcagaaaatcctgaacgtgcagaagaagctgcccatcatccagaaaatcatcatcatggacagcaagaccgactaccagggcttccagagcatgtacaccttcgtgaccagccatctgccacctggcttcaacgagtacgacttcgtgcccgagagcttcgaccgggacaagacaatcgccctgatcatgaacagcagcggctctaccggactgcccaaaggtgttgccctgcctcatagaactgcctgcgtgcggtttagccacgccagagatcccatcttcggcaaccagatcatccccgacacagccatcctgagcgtggtgccttttcaccacggcttcggcatgttcaccacactgggctacctgatctgcggcttcagagtggtgctgatgtaccgcttcgaggaagaactgttcctgcggagcctgcaggactacaagatccagtctgctctgctggtgcctactctgttcagcttctttgccaagagcaccctgatcgataagtacgacctgagcaacctgcacgagatcgcctctggcggagcccctctgtctaaagaagtgggagaagccgtcgccaagcggttccatctgcctggcatcagacaaggctatggcctgaccgagacaaccagcgccattctgattacccctgagggcgacgataagcctggcgctgtgggaaaagtggtgccattcttcgaggccaaggtggtggatctggacaccggcaaaacactgggcgttaaccagaggggcgagctgtgtgttagaaaataatttctccttaaagaagaaattctttaagtggatgctctcaaactcagggaaacctaaatctagttatagacaaggcaatcctgagccaagccgaagtagtaattagtaagaccagtggacaatcgacggataacagcata

SEQ ID NO: 3 - RNA, 2711 nt, synthetic construct
  source 1..2711: mol_type = other RNA
taatacgactcactatagggccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgcatcatggacagcaagaccgactaccagggcttccagagcatgtacaccttcgtgaccagccatctgccacctggcttcaacgagtacgacttcgtgcccgagagcttcgaccgggacaagacaatcgccctgatcatgaacagcagcggctctaccggactgcccaaaggtgttgccctgcctcatagaactgcctgcgtgcggtttagccacgccagagatcccatcttcggcaaccagatcatccccgacacagccatcctgagcgtggtgccttttcaccacggcttcggcatgttcaccacactgggctacctgatctgcggcttcagagtggtgctgatgtaccgcttcgaggaagaactgttcctgcggagcctgcaggactacaagatccagtctgctctgctggtgcctactctgttcagcttctttgccaagagcaccctgatcgataagtacgacctgagcaacctgcacgagatcgcctctggcggagcccctctgtctaaagaagtgggagaagccgtcgccaagcggttccatctgcctggcatcagacaaggctatggcctgaccgagacaaccagcgccattctgattacccctgagggcgacgataagcctggcgctgtgggaaaagtggtgccattcttcgaggccaaggtggtggatctggacaccggcaaaacactgggcgttaaccagaggggcgagctgtgtgttagaggccctatgatcatgagcggctacgtgaacaaccccgaggccacaaacgctctgatcgacaaggatggatggctgcacagcggcgacattgcctactgggacgaagatgagcacttcttcatcgtggaccggctgaagtccctgatcaagtacaagggctaccaggtggccccagccgagctggaatctattctgctgcaacaccccaacatcttcgatgccggcgttgcaggactgcccgatgatgatgctggcgaactgccagctgctgtggtggtgctggaacacggcaagaccatgaccgagaaagaaatcgtggactacgtggccagccaagtgaccaccgccaagaaactgagaggcggcgtggtgtttgtggacgaggtgccaaaaggcctgacaggcaagctggacgcccggaagatcagagagatcctgattaaggccaagaaaggcggcaagatcgccgtgtgattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggaagatgccaagaacatcaagaagggccctgctccattctaccctctggaagatggaacagccggcgagcagctgcacaaggccatgaagagatatgccctggtgcctggcacaatcgccttcacagatgcccacatcgaggtggacatcacctacgccgagtacttcgagatgtctgtgcggctggccgaagctatgaagcgctacggcctgaacaccaaccaccggatcgtcgtgtgcagcgagaacagcctgcagttcttcatgcctgtgctgggcgccctgttcatcggagttgctgtggcccctgccaacgacatctacaacgagcgcgagctgctgaacagcatgggcatctctcagcccaccgtggtgttcgtgtccaagaagggactgcagaaaatcctgaacgtgcagaagaagctgcccatcatccagaaaatcataaataattgtgccttaaagaagaaattctttaagtggatgctctcaaactcagggaaacctaaatctagttatagacaaggcaatcctgagccaagccgaagtagtaattagtaagaccagtggacaatcgacggataacagcata

SEQ ID NO: 4 - RNA, 2711 nt, synthetic construct
  source 1..2711: mol_type = other RNA
taatacgactcactatagggccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgtttcaccacggcttcggcatgttcaccacactgggctacctgatctgcggcttcagagtggtgctgatgtaccgcttcgaggaagaactgttcctgcggagcctgcaggactacaagatccagtctgctctgctggtgcctactctgttcagcttctttgccaagagcaccctgatcgataagtacgacctgagcaacctgcacgagatcgcctctggcggagcccctctgtctaaagaagtgggagaagccgtcgccaagcggttccatctgcctggcatcagacaaggctatggcctgaccgagacaaccagcgccattctgattacccctgagggcgacgataagcctggcgctgtgggaaaagtggtgccattcttcgaggccaaggtggtggatctggacaccggcaaaacactgggcgttaaccagaggggcgagctgtgtgttagaggccctatgatcatgagcggctacgtgaacaaccccgaggccacaaacgctctgatcgacaaggatggatggctgcacagcggcgacattgcctactgggacgaagatgagcacttcttcatcgtggaccggctgaagtccctgatcaagtacaagggctaccaggtggccccagccgagctggaatctattctgctgcaacaccccaacatcttcgatgccggcgttgcaggactgcccgatgatgatgctggcgaactgccagctgctgtggtggtgctggaacacggcaagaccatgaccgagaaagaaatcgtggactacgtggccagccaagtgaccaccgccaagaaactgagaggcggcgtggtgtttgtggacgaggtgccaaaaggcctgacaggcaagctggacgcccggaagatcagagagatcctgattaaggccaagaaaggcggcaagatcgccgtgtgattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggaagatgccaagaacatcaagaagggccctgctccattctaccctctggaagatggaacagccggcgagcagctgcacaaggccatgaagagatatgccctggtgcctggcacaatcgccttcacagatgcccacatcgaggtggacatcacctacgccgagtacttcgagatgtctgtgcggctggccgaagctatgaagcgctacggcctgaacaccaaccaccggatcgtcgtgtgcagcgagaacagcctgcagttcttcatgcctgtgctgggcgccctgttcatcggagttgctgtggcccctgccaacgacatctacaacgagcgcgagctgctgaacagcatgggcatctctcagcccaccgtggtgttcgtgtccaagaagggactgcagaaaatcctgaacgtgcagaagaagctgcccatcatccagaaaatcatcatcatggacagcaagaccgactaccagggcttccagagcatgtacaccttcgtgaccagccatctgccacctggcttcaacgagtacgacttcgtgcccgagagcttcgaccgggacaagacaatcgccctgatcatgaacagcagcggctctaccggactgcccaaaggtgttgccctgcctcatagaactgcctgcgtgcggtttagccacgccagagatcccatcttcggcaaccagatcatccccgacacagccatcctgagcgtggtgcctaaataattgggccttaaagaagaaattctttaagtggatgctctcaaactcagggaaacctaaatctagttatagacaaggcaatcctgagccaagccgaagtagtaattagtaagaccagtggacaatcgacggataacagcata

SEQ ID NO: 5 - RNA, 2809 nt, synthetic construct
  source 1..2809: mol_type = other RNA
ccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatcgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggaagatgccaagaacatcaagaagggccctgctccattctaccctctggaagatggaacagccggcgagcagctgcacaaggccatgaagagatatgccctggtgcctggcacaatcgccttcacagatgcccacatcgaggtggacatcacctacgccgagtacttcgagatgtctgtgcggctggccgaagctatgaagcgctacggcctgaacaccaaccaccggatcgtcgtgtgcagcgagaacagcctgcagttcttcatgcctgtgctgggcgccctgttcatcggagttgctgtggcccctgccaacgacatctacaacgagcgcgagctgctgaacagcatgggcatctctcagcccaccgtggtgttcgtgtccaagaagggactgcagaaaatcctgaacgtgcagaagaagctgcccatcatccagaaaatcatcatcatggacagcaagaccgactaccagggcttccagagcatgtacaccttcgtgaccagccatctgccacctggcttcaacgagtacgacttcgtgcccgagagcttcgaccgggacaagacaatcgccctgatcatgaacagcagcggctctaccggactgcccaaaggtgttgccctgcctcatagaactgcctgcgtgcggtttagccacgccagagatcccatcttcggcaaccagatcatccccgacacagccatcctgagcgtggtgccttttcaccacggcttcggcatgttcaccacactgggctacctgatctgcggcttcagagtggtgctgatgtaccgcttcgaggaagaactgttcctgcggagcctgcaggactacaagatccagtctgctctgctggtgcctactctgttcagcttctttgccaagagcaccctgatcgataagtacgacctgagcaacctgcacgagatcgcctctggcggagcccctctgtctaaagaagtgggagaagccgtcgccaagcggttccatctgcctggcatcagacaaggctatggcctgaccgagacaaccagcgccattctgattacccctgagggcgacgataagcctggcgctgtgggaaaagtggtgccattcttcgaggccaaggtggtggatctggacaccggcaaaacactgggcgttaaccagaggggcgagctgtgtgttagaggccctatgatcatgagcggctacgtgaacaaccccgaggccacaaacgctctgatcgacaaggatggatggctgcacagcggcgacattgcctactgggacgaagatgagcacttcttcatcgtggaccggctgaagtccctgatcaagtacaagggctaccaggtggccccagccgagctggaatctattctgctgcaacaccccaacatcttcgatgccggcgttgcaggactgcccgatgatgatgctggcgaactgccagctgctgtggtggtgctggaacacggcaagaccatgaccgagaaagaaatcgtggactacgtggccagccaagtgaccaccgccaagaaactgagaggcggcgtggtgtttgtggacgaggtgccaaaaggcctgacaggcaagctggacgcccggaagatcagagagatcctgattaaggccaagaaaggcggcaagatcgccgtgtgaaaaaaacaaaaaacaaaacggctattatgcgttaccggcgcttaaataattgagccttaaagaagaaattctttaagtggatgctctcaaactcagggaaacctaaatctagttatagacaaggcaatcctgagccaagccgaagtagtaattagtaagaccagtggacaatcgacggataacagcata

SEQ ID NO: 6 - RNA, 2014 nt, synthetic construct
  source 1..2014: mol_type = other RNA
taatacgactcactatagggccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggctcgctttcttgctgtccaatttctattaaaggttcctttgttccctaagtccaactactaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcata

SEQ ID NO: 7 - RNA, 1838 nt, synthetic construct
  source 1..1838: mol_type = other RNA
taatacgactcactatagggccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggctcgctttcttgctgtccaatttctattaaaggttcctttgttccctaagtccaactactaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtgagcaagggcgaggagctgttcaccggggtggtgcccatcctggtcgagctggacggcgacgtaaataattgcgccttaaagaagaaattctttaagtggatgctctcaaactcagggaaacctaaatctagttatagacaaggcaatcctgagccaagccgaagtagtaattagtaagaccagtggacaatcgacggataacagcata

SEQ ID NO: 8 - RNA, 50 nt, synthetic construct
  source 1..50: mol_type = other RNA
ggagtgctagaggatgaccttaaatcagggacgatattataattgactaa

SEQ ID NO: 9 - RNA, 51 nt, synthetic construct
  source 1..51: mol_type = other RNA
actcactatttgttttcgcgcccagttgcaaaaagtgtcgtaattgactaa

SEQ ID NO: 10 - RNA, 56 nt, synthetic construct
  source 1..56: mol_type = other RNA
ccggcggccggcggccggcggccggcggccggcggccggcggccggcggccggcgg

SEQ ID NO: 11 - RNA, 54 nt, synthetic construct
  source 1..54: mol_type = other RNA
ccggcgggtccggcgggtccggcgggtccggcgggtccggcgggtccggcgggt

SEQ ID NO: 12 - RNA, 56 nt, synthetic construct
  source 1..56: mol_type = other RNA
tactccctactccctactccctactccctactccctactccctactccctactccc

SEQ ID NO: 13 - RNA, 100 nt, synthetic construct
  source 1..100: mol_type = other RNA
cattctgtggtctgatcatcctgtggtttcgtcgccgccatcctcgtcgcgacacgctgttttcggttctcggcccgacgagccatcgccatcctacagc

SEQ ID NO: 14 - RNA, 100 nt, synthetic construct
  source 1..100: mol_type = other RNA
cactcgcgctgccatcactcttccgccgtcttcgccgccatcctcggcgcgactcgcttctttcggttctaccaggtagagtccgccgccatcctccacc

SEQ ID NO: 15 - RNA, 100 nt, synthetic construct
  source 1..100: mol_type = other RNA
cttgtctcgctccggggaacgctcggaaactcccggccgccgccacccgcgtctgttctgttacacaagggaagaaaagccgctgccgcactccgagtgt

SEQ ID NO: 16 - RNA, 40 nt, synthetic construct
  source 1..40: mol_type = other RNA
aaaaaaaaaaaaaaaaaaaaaaaaaaaaaaacccccccca

SEQ ID NO: 17 - RNA, 50 nt, synthetic construct
  source 1..50: mol_type = other RNA
aaaaaaaaaaaaaaaaaaaaaaaaaaaaaaaaaaaaaaaaacccccccca

SEQ ID NO: 18 - RNA, 60 nt, synthetic construct
  source 1..60: mol_type = other RNA
aaaaaaaaaaaaaaaaaaaaaaaaaaaaaaaaaaaaaaaaaaaaaaaaaaacccccccca

SEQ ID NO: 19 - RNA, 70 nt, synthetic construct
  source 1..70: mol_type = other RNA
aaaaaaaaaaaaaaaaaaaaaaaaaaaaaaaaaaaaaaaaaaaaaaaaaaaaaaaaaaaaacccccccca

SEQ ID NO: 20 - RNA, 80 nt, synthetic construct
  source 1..80: mol_type = other RNA
aaaaaaaaaaaaaaaaaaaaaaaaaaaaaaaaaaaaaaaaaaaaaaaaaaaaaaaaaaaaaaaaaaaaaaacccccccca

SEQ ID NO: 21 - RNA, 90 nt, synthetic construct
  source 1..90: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaacccccccca

SEQ ID NO: 22 - RNA, 50 nt, synthetic construct
  source 1..50: mol_type = other RNA
aacggaacgtatattcaacgcaatgaagccggaggattggcgtgggaatc

SEQ ID NO: 23 - RNA, 50 nt, synthetic construct
  source 1..50: mol_type = other RNA
taatatttatatatttatatttttaaaatatttatttatttatttattta

SEQ ID NO: 24 - RNA, 64 nt, synthetic construct
  source 1..64: mol_type = other RNA
cctcgccccggacctgccctcccgccaggtgcacccacctgcaataaatgcagcgaagccggga

SEQ ID NO: 25 - RNA, 102 nt, synthetic construct
  source 1..102: mol_type = other RNA
gaccactttggccttcatcttggttacagaaaagggaatagaaatgaaacaaattatctctcatcttttgactatttcaagtctaataaattcttaattaac

SEQ ID NO: 26 - RNA, 210 nt, synthetic construct
  source 1..210: mol_type = other RNA
gacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaaccaggagctccc

SEQ ID NO: 27 - RNA, 101 nt, synthetic construct
  source 1..101: mol_type = other RNA
gctggagcctcggtggccatgcttcttgccccttgggcctccccccagcccctcctccccttcctgcacccgtacccccgtggtctttgaataaagtctga

SEQ ID NO: 28 - RNA, 107 nt, synthetic construct
  source 1..107: mol_type = other RNA
gctcgctttcttgctgtccaatttctattaaaggttcctttgttccctaagtccaactactaaactgggggatattatgaagggccttgagcatctggattctgcct

SEQ ID NO: 29 - RNA, 56 nt, synthetic construct; identical to SEQ ID NO: 12
  source 1..56: mol_type = other RNA

SEQ ID NO: 30 - RNA, 100 nt, synthetic construct; identical to SEQ ID NO: 14
  source 1..100: mol_type = other RNA

SEQ ID NO: 31 - RNA, 20 nt, synthetic construct
  source 1..20: mol_type = other RNA
aaaaaaaaaaacccccccca

SEQ ID NO: 32 - RNA, 24 nt, synthetic construct
  source 1..24: mol_type = other RNA
aaaaaaaaaaaaaaacccccccca

SEQ ID NO: 33 - RNA, 30 nt, synthetic construct
  source 1..30: mol_type = other RNA
aaaaaaaaaaaaaaaaaaaaacccccccca

SEQ ID NO: 34 - RNA, 40 nt, synthetic construct; identical to SEQ ID NO: 16
  source 1..40: mol_type = other RNA

SEQ ID NO: 35 - RNA, 50 nt, synthetic construct; identical to SEQ ID NO: 17
  source 1..50: mol_type = other RNA

SEQ ID NO: 36 - RNA, 80 nt, synthetic construct; identical to SEQ ID NO: 20
  source 1..80: mol_type = other RNA

SEQ ID NO: 37 - RNA, 2872 nt, synthetic construct
  source 1..2872: mol_type = other RNA
taatacgactcactatagggccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaagggaaaaaaaaaaaaaaaaaaaaaaaaaaaaaaaaaaaaaaaaaaaaaaaaaaaaaaaaaaaaaccccccccattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggaagatgccaagaacatcaagaagggccctgctccattctaccctctggaagatggaacagccggcgagcagctgcacaaggccatgaagagatatgccctggtgcctggcacaatcgccttcacagatgcccacatcgaggtggacatcacctacgccgagtacttcgagatgtctgtgcggctggccgaagctatgaagcgctacggcctgaacaccaaccaccggatcgtcgtgtgcagcgagaacagcctgcagttcttcatgcctgtgctgggcgccctgttcatcggagttgctgtggcccctgccaacgacatctacaacgagcgcgagctgctgaacagcatgggcatctctcagcccaccgtggtgttcgtgtccaagaagggactgcagaaaatcctgaacgtgcagaagaagctgcccatcatccagaaaatcatcatcatggacagcaagaccgactaccagggcttccagagcatgtacaccttcgtgaccagccatctgccacctggcttcaacgagtacgacttcgtgcccgagagcttcgaccgggacaagacaatcgccctgatcatgaacagcagcggctctaccggactgcccaaaggtgttgccctgcctcatagaactgcctgcgtgcggtttagccacgccagagatcccatcttcggcaaccagatcatccccgacacagccatcctgagcgtggtgccttttcaccacggcttcggcatgttcaccacactgggctacctgatctgcggcttcagagtggtgctgatgtaccgcttcgaggaagaactgttcctgcggagcctgcaggactacaagatccagtctgctctgctggtgcctactctgttcagcttctttgccaagagcaccctgatcgataagtacgacctgagcaacctgcacgagatcgcctctggcggagcccctctgtctaaagaagtgggagaagccgtcgccaagcggttccatctgcctggcatcagacaaggctatggcctgaccgagacaaccagcgccattctgattacccctgagggcgacgataagcctggcgctgtgggaaaagtggtgccattcttcgaggccaaggtggtggatctggacaccggcaaaacactgggcgttaaccagaggggcgagctgtgtgttagaggccctatgatcatgagcggctacgtgaacaaccccgaggccacaaacgctctgatcgacaaggatggatggctgcacagcggcgacattgcctactgggacgaagatgagcacttcttcatcgtggaccggctgaagtccctgatcaagtacaagggctaccaggtggccccagccgagctggaatctattctgctgcaacaccccaacatcttcgatgccggcgttgcaggactgcccgatgatgatgctggcgaactgccagctgctgtggtggtgctggaacacggcaagaccatgaccgagaaagaaatcgtggactacgtggccagccaagtgaccaccgccaagaaactgagaggcggcgtggtgtttgtggacgaggtgccaaaaggcctgacaggcaagctggacgcccggaagatcagagagatcctgattaaggccaagaaaggcggcaagatcgccgtgtgaaaaaaaaaaaaaaaaaaaaaaaaaaaaaaaaaaaaaaaaaaaaaaaaaaaaaaaaaaaaaaaaaaaaaaaaccccccccaccccttaaataattgagccttaaagaagaaattctttaagtggatgctctcaaactcagggaaacctaaatctagttatagacaaggcaatcctgagccaagccgaagtagtaattagtaagaccagtggacaatcgacggataacagcata

SEQ ID NO: 38 - RNA, 5075 nt, synthetic construct
  source 1..5075: mol_type = other RNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagagaattcgagctcggtacctcgcgaatacatctagataatacgactcactatagggccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggaagatgccaagaacatcaagaagggccctgctccattctaccctctggaagatggaacagccggcgagcagctgcacaaggccatgaagagatatgccctggtgcctggcacaatcgccttcacagatgcccacatcgaggtggacatcacctacgccgagtacttcgagatgtctgtgcggctggccgaagctatgaagcgctacggcctgaacaccaaccaccggatcgtcgtgtgcagcgagaacagcctgcagttcttcatgcctgtgctgggcgccctgttcatcggagttgctgtggcccctgccaacgacatctacaacgagcgcgagctgctgaacagcatgggcatctctcagcccaccgtggtgttcgtgtccaagaagggactgcagaaaatcctgaacgtgcagaagaagctgcccatcatccagaaaatcatcatcatggacagcaagaccgactaccagggcttccagagcatgtacaccttcgtgaccagccatctgccacctggcttcaacgagtacgacttcgtgcccgagagcttcgaccgggacaagacaatcgccctgatcatgaacagcagcggctctaccggactgcccaaaggtgttgccctgcctcatagaactgcctgcgtgcggtttagccacgccagagatcccatcttcggcaaccagatcatccccgacacagccatcctgagcgtggtgccttttcaccacggcttcggcatgttcaccacactgggctacctgatctgcggcttcagagtggtgctgatgtaccgcttcgaggaagaactgttcctgcggagcctgcaggactacaagatccagtctgctctgctggtgcctactctgttcagcttctttgccaagagcaccctgatcgataagtacgacctgagcaacctgcacgagatcgcctctggcggagcccctctgtctaaagaagtgggagaagccgtcgccaagcggttccatctgcctggcatcagacaaggctatggcctgaccgagacaaccagcgccattctgattacccctgagggcgacgataagcctggcgctgtgggaaaagtggtgccattcttcgaggccaaggtggtggatctggacaccggcaaaacactgggcgttaaccagaggggcgagctgtgtgttagaggccctatgatcatgagcggctacgtgaacaaccccgaggccacaaacgctctgatcgacaaggatggatggctgcacagcggcgacattgcctactgggacgaagatgagcacttcttcatcgtggaccggctgaagtccctgatcaagtacaagggctaccaggtggccccagccgagctggaatctattctgctgcaacaccccaacatcttcgatgccggcgttgcaggactgcccgatgatgatgctggcgaactgccagctgctgtggtggtgctggaacacggcaagaccatgaccgagaaagaaatcgtggactacgtggccagccaagtgaccaccgccaagaaactgagaggcggcgtggtgtttgtggacgaggtgccaaaaggcctgacaggcaagctggacgcccggaagatcagagagatcctgattaaggccaagaaaggcggcaagatcgccgtgtgaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcataagaagagcacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 39 - RNA, 27 nt, synthetic construct
  source 1..27: mol_type = other RNA
cggaaggtgcagagactcgacgggagc

SEQ ID NO: 40 - RNA, 28 nt, synthetic construct
  source 1..28: mol_type = other RNA
acgtcaagacgagggtaaagagagagtc

SEQ ID NO: 41 - RNA, 29 nt, synthetic construct
  source 1..29: mol_type = other RNA
caaggcaatcctgagccaagccgaagtag

SEQ ID NO: 42 - RNA, 28 nt, synthetic construct
  source 1..28: mol_type = other RNA
tgctctcaaactcagggaaacctaaatc

SEQ ID NO: 43 - RNA, 264 nt, synthetic construct
  source 1..264: mol_type = other RNA
acggacttaaataattgagccttaaagaagaaattctttaagtggatgctctcaaactcagggaaacctaaatctagttatagacaaggcaatcctgagccaagccgaagtagtaattagtaagaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgt

SEQ ID NO: 44 - RNA, 131 nt, synthetic construct
  source 1..131: mol_type = other RNA
aacaatagatgacttacaactaatcggaaggtgcagagactcgacgggagctaccctaacgtcaagacgagggtaaagagagagtccaattctcaaagccaataggcagtagcgaaagctgcaagagaatg

SEQ ID NO: 45 - RNA, 115 nt, synthetic construct
  source 1..115: mol_type = other RNA
aaataattgagccttaaagaagaaattctttaagtggatgctctcaaactcagggaaacctaaatctagttatagacaaggcaatcctgagccaagccgaagtagtaattagtaa

SEQ ID NO: 46 - DNA, 20 bp, synthetic construct
  source 1..20: mol_type = other DNA
gtttcattttattcctatac

SEQ ID NO: 47 - DNA, 28 bp, synthetic construct
  source 1..28: mol_type = other DNA
caattgagagatcgttaccatatagcta

SEQ ID NO: 48 - DNA, 12 bp, synthetic construct
  source 1..12: mol_type = other DNA
attttattccta

SEQ ID NO: 49 - DNA, 18 bp, synthetic construct
  source 1..18: mol_type = other DNA
ccggcgggtccggcgggt

SEQ ID NO: 50 - DNA, 45 bp, synthetic construct
  source 1..45: mol_type = other DNA
ccggcgggtccggcgggtccggcgggtccggcgggtccggcgggt

SEQ ID NO: 51 - DNA, 40 bp, synthetic construct
  source 1..40: mol_type = other DNA
gtttcattttattcctatacgtttcattttattcctatac

(19) United States
(12) Patent Application Publication
(10) Pub. No.: US 2026/0357287 A1
(43) Pub. Date: Nov. 12, 2026

(54) UNIVERSAL SCAR-FREE RNA CIRCULARIZATION METHOD

(71) Applicant: PROXYBIO THERAPEUTICS CO., LTD., Shenzhen (CN)

(72) Inventors: Jiaqi LIN, Shenzhen (CN); Bangda FAN, Shenzhen (CN)

(73) Assignee: PROXYBIO THERAPEUTICS CO., LTD., Shenzhen (CN)

(21) Appl. No.: 19/682,381

(22) Filed: May 19, 2026

Related U.S. Application Data
(63) Continuation of application No. PCT/CN2025/071777, filed on Jan. 10, 2025.

(30) Foreign Application Priority Data
Jan. 11, 2024 (CN) 202410046331.2

(51) Int. Cl. C12N 15/85; C07K 14/435; C12N 9/02; C12N 15/113

(52) U.S. Cl. CPC C12N 15/85; C07K 14/43595; C12N 9/0069; C12N 15/113; C12Y 113/12007; C12N 2310/12; C12N 2310/532; C12N 2830/50; C12N 2840/203

(57) ABSTRACT

Provided is a universal scar-free RNA circularization method, including a recombinant nucleic acid molecule for preparing a circular RNA, in a 5′ to 3′ direction, the recombinant nucleic acid molecule includes elements arranged in the following order: an optional 5′ homology arm, a 3′ half-intron fragment, a circularization fragment, a 5′ half-intron fragment, and an optional 3′ homology arm; and the circularization fragment includes a 3′ circularization recognition segment at a 5′ and a 5′ circularization recognition segment at a 3′.

### CROSS-REFERENCE TO RELATED APPLICATIONS

This application is a continuation of International Application No. PCT/CN2025/071777, filed on Jan. 10, 2025, which claims priority to Chinese Patent Application No. 202410046331.2, filed on Jan. 11, 2024, the entire contents of each of which are incorporated herein by reference.

### SEQUENCE LISTING

The instant application contains a Sequence Listing which is submitted electronically in XML format and is hereby incorporated by reference in its entirety. The XML copy, created on_Jul. 16, 2026, is named “2026-07-16-Sequence Listing-65622-H062US00” and is 73,506 bytes in size.

### TECHNICAL FIELD

The present disclosure belongs to the field of biochemistry, and relates to a universal scar-free RNA circularization method.

### BACKGROUND

In recent years, researchers have found that circular RNA prepared by the Permuted Intron-Exon (PIE) method have strong immunogenicity. For example, Howard Y Chang et al. found in 2017 and 2019 that circular RNA prepared by the PIE method had strong immunogenicity in cells. Through the determination of cytokines such as RIG-I, it was found that the immunogenicity intensity induced by circular RNA prepared by the PIE method was comparable to that of unmodified linear messenger RNA (mRNA). It was found that the immunogenicity was caused by an unavoidable insertion of a foreign exon sequence into the RNA during the preparation of the circular RNA using the PIE method. In addition, Ling-Ling Chen et al. also found a same phenomenon and that a source of the immunogenicity was a “scar” sequence introduced during the preparation by using the PIE method. Further studies indicated that the immunogenicity was induced by an unconventional duplex structure formed by the “scar” sequence. Therefore, from the above research, although the PIE method has a high circularization efficiency, it has a disadvantage of inducing strong immunogenicity, which greatly hinders the application of the circular RNA. The immunogenicity is mainly caused by the “scar” sequence. In addition, more and more literature has found that the circular RNA has a regulatory function and can serve as a potential therapeutic molecule. For the synthesis of a therapeutic circular RNA molecule, if the PIE method is used, an extra sequence of nearly 200 nt including a “scar” fragment and a spacer sequence is introduced. This obviously affects the biological activity of such a therapeutic circular RNA molecule.

Therefore, it is desirable to provide a universal scar-free circularization method, which can maintain excellent properties of the PIE method, avoid high immunogenicity, and also serve as a method for preparing therapeutic circular RNA in vitro. In a subsequent design, this system is designed as a universal system, and different sequences to be circularized can be used without special design, which is more convenient.

### SUMMARY

One or more embodiments of the present disclosure provide a recombinant nucleic acid molecule for preparing circular RNA, wherein in a 5′ to 3′ direction, the recombinant nucleic acid molecule comprises elements operably linked in sequence, the elements comprising an optional 5′ homology arm, a 3′ half-intron fragment, a circularization fragment, a 5′ half-intron fragment, and an optional 3′ homology arm; wherein a 5′ of the circularization fragment comprises a 3′ circularization recognition segment, and a 3′ of the circularization fragment comprises a 5′ circularization recognition segment; the 5′ half-intron fragment and the 3′ half-intron fragment are derived from a group I intron, and the group I intron is derived from Anabaena, specifically, Anabaena tRNA^{leu}; the 5′ half-intron fragment and the 3′ half-intron fragment form an intron sequence in the 5′ to 3′ direction; a nucleotide sequence of the 5′ half-intron fragment comprises a part of the intron sequence proximal to the 5′, a nucleotide sequence of the 3′ half-intron fragment comprises a remaining part of the intron sequence proximal to the 3′, and the 5′ half-intron fragment comprises an internal guide (IG) sequence of the intron sequence; wherein (a) the IG sequence of the intron sequence is 3 nt of 5′-GNN-3′, the 5′ circularization recognition segment is 3 nt of 5′-NNY-3′, wherein Y is C or T/U; N is any nucleotide, and the IG sequence and bases corresponding to the 5′ circularization recognition segment have strict complementary base pairing or GU wobble pairing; (b) the 5′ of the circularization fragment comprises at least 2 nt of the 3′ circularization recognition segment, and the 3′ circularization recognition segment does not have significant homology with the 5′ circularization recognition segment and adjacent nucleotide sequences; and (c) an adjacent nucleotide sequence of the 5′ circularization recognition segment and an adjacent nucleotide sequence of the 3′ circularization recognition segment comprise at least 3 nt of an internal homologous sequence; wherein the significant homology refers to complementary hase pairing between two or more nucleotides.

One or more embodiments of the present disclosure provide a recombinant expression vector, wherein the recombinant expression vector comprises the recombinant nucleic acid molecule for preparing circular RNA.

One or more embodiments of the present disclosure provide a method for preparing circular RNA in vitro, comprising: 1) transcribing a recombinant expression vector to form a circularization precursor nucleic acid molecule; 2) subjecting the circularization precursor nucleic acid molecule to a circularization reaction to obtain a circular RNA; and 3) purifying the circular RNA.

One or more embodiments of the present disclosure provide a composition, wherein the composition comprises the recombinant nucleic acid molecule for preparing circular RNA, the recombinant expression vector comprising the recombinant nucleic acid molecule, or the circular RNA prepared based on the recombinant nucleic acid molecule; and one or more pharmaceutically acceptable carriers; wherein the pharmaceutically acceptable carriers include at least one of a lipid, a polymer, or a lipid-polymer complex.

One or more embodiments of the present disclosure provide a method for preventing or treating a disease, wherein the method comprises administering to a subject the circular RNA, or a composition comprising the circular RNA.

One or more embodiments of the present disclosure provide a design method for a recombinant nucleic acid molecule for preparing circular RNA, wherein the method comprises: 1) providing a candidate target polypeptide coding region or a non-coding region, a translation initiation element, and an optional 5′ spacer sequence and an optional 3′ spacer sequence; 2) searching or setting a 5′ circularization recognition segment, a 3′ circularization recognition segment, and an internal homologous sequence in the target polypeptide coding region or the non-coding region, the translation initiation element, or the 5′ spacer sequence and the 3′ spacer sequence, thereby designing a circularization fragment; wherein a process of the searching includes: S-1) searching for a 5′ internal homologous sequence-5′ circularization recognition segment-3′ circularization recognition segment-3′ internal homologous sequence in the target polypeptide coding region or the non-coding region, the translation initiation element, or the 5′ spacer sequence and the 3′ spacer sequence; S-2) cleaving a corresponding element at a junction between the 5′ circularization recognition segment and the 3′ circularization recognition segment according to a search result to form a circularization sequence; a process of the setting includes: setting, at two ends of a sequence: 5′ spacer sequence-translation initiation element-target polypeptide coding region or non-coding region-3′ spacer sequence, a 5′ internal homologous sequence-5′ circularization recognition segment sequence and a 3′ circularization recognition segment-3′ internal homologous sequence to form the circularization fragment; wherein A) the 5′ internal homologous sequence and the 3′ internal homologous sequence are reverse complementary sequences comprising at least 3 nt; B) the 3′ circularization recognition segment is a sequence of at least 2 nt, and does not have significant homology with a sequence adjacent to the 5′ circularization recognition segment; C) the 5′ circularization recognition segment is a 3 nt sequence, and is NNY, wherein N is any base, Y is C or T/U; 3) modifying, according to the circularization fragment, an internal guide sequence in an intron sequence to be used, wherein the internal guide sequence is 3 nt of “GNN”, and has strict complementary base pairing or GU wobble pairing with a nucleotide corresponding to the 5′ circularization recognition segment; 4) splitting the modified intron sequence into a 3′ half-intron fragment and a 5′ half-intron fragment, and operably linking an optional 5′ homology arm, the 3′ half-intron fragment, the circularization fragment, the 5′ half-intron fragment, and an optional 3′ homology arm in order to form a recombinant nucleic acid molecule for preparing circular RNA; wherein a 5′ of the circularization fragment comprises the 3′ circularization recognition segment, and a 3′ comprises the 5′ circularization recognition segment; to form the recombinant nucleic acid molecule for preparing circular RNA; the setting operation is: inserting 3 nucleotides at a third or fourth nucleotide position of the 5′ spacer sequence as the internal homologous sequence, and using two or three nucleotides of the 5′ spacer sequence itself as the 3′ circularization recognition segment; inserting 6 nucleotides at a 3′ of the 3′ spacer sequence, wherein the 1st-3rd nucleotides are complementary to the nucleotides inserted into the 5′ spacer sequence, and the 4th-6th sequence is NNY and has strict complementary base pairing or GU wobble pairing with a nucleotide corresponding to the internal guide sequence; the setting operation is: inserting 5 nucleotides at a 5′ of the 5′ spacer sequence, wherein the 1st-2nd nucleotides serve as the 3′ circularization recognition segment, the 3rd-5th nucleotides serve as the internal homologous sequence, and using two or three nucleotides of the 5′ spacer sequence itself as the 3′ circularization recognition segment; inserting 6 nucleotides at a 3′ of the 3′ spacer sequence, wherein the 1st-3rd nucleotides are complementary to the nucleotides inserted into the 5′ spacer sequence, and the 4th-6th sequence is NNY and has strict complementary base pairing or GU wobble pairing with a nucleotide corresponding to the internal guide sequence.

One or more embodiments of the present disclosure provide an internal ribosome entry site (IRES) enhancer element for insertion into a CVB3 IRES sequence to enhance translation efficiency, wherein the IRES enhancer element is inserted between domain I and domain II of an IRES sequence (designated IRES-1), at a stem-loop structure of domain II of the IRES sequence (designated IRES-2), at a distal loop of domain IV of the IRES sequence (designated IRES-3), or at a proximal loop of domain IV of the IRES sequence (designated IRES-4); and the IRES enhancer element is a sequence set forth below:

| Serial | |
|---|---|
| No. | |
| NO: | IRES sequence |
| A1 | CCGGCGGGT |
| A2 | GTTTCATTTTATTCCTATAC |
| | (SEQ ID NO: 46) |
| A3 | CAATTGAGAGATCGTTACCA |
| | TATAGCTA (SEQ ID NO: 47) |
| A4 | ATTTTATTCCTA (SEQ ID NO: 48) |

or a repeat sequence of the above sequence:

| Serial | IRES repeat sequence |
|---|---|
| A1-X2 | CCGGCGGGTCCGGCGGGT |
| | (SEQ ID NO: 49) |
| A1-X5 | CCGGCGGGTCCGGCGGGTCCGGC |
| | GGGTCCGGCGGGTCCGGCGGGT |
| | (SEQ ID NO: 50) |
| A2-X2 | GTTTCATTTTATTCCTATACGTT |
| | TCATTTTATTCCTATAC |
| | (SEQ ID NO: 51) |

One or more embodiments of the present disclosure provide a mutant or a fragment combination derived from the mutant having self-splicing activity, wherein the mutant is derived from an Anabaena tRNA^{leu} group I intron, and the mutant comprises at least one of the following mutations: 1) a second base of an original IG sequence 5′-GAG-3′ is mutated to a base other than A; and/or 2) a third base (3′ G) of the original IG sequence 5′-GAG-3′ is mutated to a base other than G; wherein the mutant comprises at least the mutation of the third base (3′ G); and the mutant is a combination of SEQ ID NOs: 44 and 45 having the mutations.

### Technical Problem Solved by the Present Disclosure

Through investigation of the molecular mechanism of the Anabaena-derived group I ribozyme, it is found that scar-free circularization can be achieved by modifying the IG sequence, while the IG sequence and the splice sites need to satisfy the following requirements:
(1) The IG sequence needs to satisfy the sequence feature of “GNN”.
(2) When the IG sequence recognizes the 5′ splice site, it must ensure complementary base pairing or “GU” wobble pairing.
(3) The 3′ splice site is not conserved, but complementary base pairing with the sequence near the 5′ splice site significantly affects circularization efficiency.
(4) The flanking splice sites need a homology arm of at least 3 nt in length to stabilize the P1 duplex.

Based on the above design principles, Firefly Luciferase (1653 nt) and eGFP (780 nt) are successfully circularized to construct a scar-free circular RNA containing only “IRES+ORF”. Separation by 2% EX-gel electrophoresis demonstrated that a high circularization efficiency is retained, enabling effective circularization of large RNA fragments. Both cell experiments and animal experiments confirm that the constructed scar-free circular RNA is successfully expressed in vivo. This demonstrates the universality and feasibility of the scar-free circularization method.

Furthermore, because circularization of different open reading frames (ORFs) requires selection of splice sites and modification of the IG sequence, this process is inconvenient. Meanwhile, experiments reveal that certain spacer sequences not only improve circularization efficiency but also enhance the translation of the circular RNA, and these spacer sequences are referred to as untranslated region (UTR) sequences. The UTR sequences are screened, and a sequence providing the best enhancement of circular RNA translation efficiency is identified, achieving a 3.7-fold increase compared to the circular RNA sequence obtained by the original PIE method. By modifying the IG sequence using the previously described scar-free circularization method, a scar-free universal circularization system containing only “IRES+ORF+UTR” is constructed. The circular RNA constructed by this method exhibits a circularization efficiency comparable to the PIE method and superior translation ability, which has been verified at both cellular and animal levels.

The present disclosure also modifies the CVB3 IRES sequence. First, the optimal position for inserting an IRES element into the CVB3 IRES sequence is investigated. Eleven positions for inserting a gene sequence are designed, and experiments show that four insertion positions yield a translation effect. The four specific insertion positions are as follows: inserting an IRES element between domain I and domain II (designated IRES-1), inserting an IRES element at a stem-loop structure of domain II (designated IRES-2), inserting an IRES element at a distal loop of domain IV (designated IRES-3), and inserting an IRES element at a proximal loop of domain IV (designated IRES-4). The IRES element can enhance the binding of the CVB3 IRES sequence to the ribosome, thereby promoting circular RNA translation.

The IRES element within the circular RNA folds into a structure similar to the initial tRNA, recruits more ribosomes, and binds translation regulatory factors such as IRES trans-acting factors (ITAFs), thereby directing the ribosome to the internal region of the circular RNA to bind and initiate protein translation. The ribosome is a highly diverse protein structure found in all cells. Under the action of translation regulatory factors such as ITAFs, the ribosome is directed to the internal structure of the circular RNA to bind and initiate translation. Second, four IRES elements are screened, and the effect of different IRES element sequences with equivalent enhancement effects on circular RNA translation efficiency is explored. Each of these four IRES elements has a ribosome recruiting function, thereby affecting circular RNA translation. The four screened IRES element sequences are individually cloned into a deoxyribonucleic acid (DNA) template. The composition of the DNA template is the same as above. Screening for the optimal sequence is performed to obtain a gene sequence for high protein expression. Finally, based on the optimal position for the IRES element and the screening of the IRES elements, a series of repeat sequences of the IRES elements are designed to obtain sequences for achieving the IRES element function. Among the screened sequences, IRES-1-A1-X2 shows an approximately 2.3-fold increase compared to the original sequence.

Finally, conditions for the in vitro transcription (IVT) reaction of circular RNA are optimized, achieving a circularization rate of greater than or equal to 75% and a naked RNA content of less than or equal to 3%; a purification method for circular nucleic acids is improved, ensuring a circular nucleic acid purity of greater than or equal to 95%, with the content of aggregates, linear precursors, introns, and isomers of less than 1%, a total purification yield of greater than or equal to 30%, and a theoretical total yield of circular nucleic acids of greater than or equal to 70%, thereby ensuring that the IVT reaction and purification of circular RNA are suitable for industrial scale-up production.

### Beneficial Technical Effects

1. Screening and modification of the Anabaena group I intron sequence. It is verified that circularization without exogenous sequences can be achieved by modifying the IG sequence to recognize the circularization sequence. The original IG sequence is “GAG”, wherein the first “G” is strictly conserved and can only recognize the 5′ splice site through “GC” pairing or “GU wobble” pairing; the second A and the third G are not conserved and can be modified according to the specific nucleotides of the circularization sequence, as long as a strict complementary base pairing relationship or wobble pairing relationship is ensured. In summary, the IG sequence of Anabaena needs to satisfy “GNN” and can be modified according to the desired circularization sequence. Furthermore, the 3′ splice site of the circularization sequence is not conserved. The Anabaena ribozyme system differs from other group I ribozyme systems in that its IG sequence does not recognize the 3′ splice site. The present disclosure also reaches the same conclusion, but further finds that the 3′ splice site must have at least 3 nt of nucleotides that are non-homologous to the 5′ splice site; otherwise, the circularization efficiency is affected. Additionally, since the Anabaena ribozyme is derived from tRNA, its original exon sequence is a “cloverleaf” stem structure, naturally having a homology of 5 nt in length. When performing circularization without exogenous sequences, it is also necessary to retain homology of a segment of sequence around the splice site. The present disclosure finds that a homology length as short as 3 nt does not affect the circularization efficiency.

The present disclosure also screens UTR sequences. Here, UTR refers to the spacer sequence in the original PIE technology, whose main function is to provide a “spacer” effect between the ribozyme and the IRES, thereby improving circularization efficiency. The present disclosure finds that UTR sequences also have the effect of both “spacing” and improving translation efficiency. In the present disclosure, ten sequences are screened for each of the 5′ UTR and the 3′ UTR. It is found that 41A8CA and 51A8CA in the 5′ UTR could improve translation efficiency, and 15A8CA in the 3′ UTR could also improve translation efficiency. It is also found that the improvement in translation efficiency is proportional to the number of A residues. Therefore, after increasing the number of A residues, the translation efficiency is further enhanced, leading to the identification of the optimal sequences: 5′61A8CA and 3′71A8CA. The present disclosure screens different 5′ and 3′ UTRs and combining the optimal UTRs at both ends improves the translation efficiency of the circular RNA by approximately 5.7-fold.

Furthermore, the present disclosure also modifies the CVB3 IRES sequence. First, the optimal position for inserting an IRES enhancer element into the CVB3 IRES sequence is investigated. Eleven positions for inserting a gene sequence are designed, and experiments show that four insertion positions yield a translation effect. The four specific insertion positions are as follows: inserting an IRES enhancer element between domain I and domain II (designated IRES-1), inserting an IRES enhancer element at a stem-loop structure of domain II (designated IRES-2), inserting an IRES enhancer element at a distal loop of domain IV (designated IRES-3), and inserting an IRES enhancer element at a proximal loop of domain IV (designated IRES-4). The IRES enhancer element can enhance the binding of the CVB3 IRES sequence to the ribosome, thereby promoting circular RNA translation. The IRES enhancer element within the circular RNA folds into a structure similar to the initial tRNA, recruits more ribosomes, and binds translation regulatory factors such as ITAFs, thereby directing the ribosome to the internal region of the circular RNA to bind and initiate protein translation. The ribosome is a highly diverse protein structure found in all cells. Under the action of translation regulatory factors such as ITAFs, the ribosome is directed to the internal structure of the circular RNA to bind and initiate translation. Second, four IRES enhancer elements are screened, and the effect of different IRES enhancer element sequences with equivalent enhancement effects on circular RNA translation efficiency is explored. Each of these four IRES enhancer elements has a ribosome recruiting function, thereby affecting circular RNA translation. The four screened IRES enhancer element sequences are individually cloned into a DNA template. The composition of the DNA template is the same as above. Screening for the optimal sequence is performed to obtain a gene sequence for high protein expression. Finally, based on the optimal position for the IRES enhancer element and the screening of the IRES enhancer elements, a series of repeat sequences of the IRES enhancer elements are designed to obtain sequences for achieving the IRES enhancer element function. Among the screened sequences, IRES-1-A1-X2 shows an approximately 2.3-fold increase compared to the original sequence.

### BRIEF DESCRIPTION OF THE DRAWINGS

FIGS. 1A-1E are design diagrams according to Example 1 of the present disclosure (FIG. 1A: main elements of the PIE method and the structure of Design1; FIG. 1B: Design2 with added “GU” wobble pairing; FIG. 1C: analysis of circular RNA generation from Design2 by 2% EX-gel electrophoresis; FIG. 1D: expression of circular RNA produced from Design2 introduced into HeLa cells; and FIG. 1E: reverse transcription Polymerase Chain Reaction (PCR) sequencing of the product from Design2);

FIGS. 2A-2D are schematic diagrams illustrating the effect of the number of bases of the 3′ circularization recognition segment on circularization efficiency according to some embodiments of the present disclosure (FIG. 2A: Trunc-Exon; FIG. 2B: reverse transcription PCR sequencing of the Trunc-Exon product; FIG. 2C: fluorescence electrophoresis of products after single nucleotide deletion of the 5 nt sequence; and FIG. 2D: cellular expression of products after single nucleotide deletion of the 5 nt sequence);

FIGS. 3A-3G are schematic diagrams illustrating the conservation test of the 2 nt sequence of the 3′ circularization recognition segment according to some embodiments of the present disclosure (FIGS. 3A-3B: construction of possible combinations based on the 2 nt structure; FIGS. 3C-3D: almost all 2 nt sequences can be successfully circularized without a decrease in circularization efficiency; FIG. 3F: homology between the 2 nt sequence and the adjacent sequence of the 5′ circularization recognition segment); and FIGS. 3E-3G: validation of the effect of homology between the sequences adjacent to the 3′ and 5′ splice sites on cyclization efficiency;

FIGS. 4A-4E are schematic diagrams illustrating that the IG sequence is partially conserved and recognizes the 5′ circularization recognition segment through base pairing or “GU” wobble pairing according to some embodiments of the present disclosure (FIG. 4A: original Exon1 sequence; FIG. 4B: mutated Exon1 sequence; FIG. 4C: IG sequence needs to satisfy the sequence feature of “GNN”; FIG. 4D: effect of “GU” wobble pairing on splicing; and FIG. 4E: failure to satisfy base pairing completely disrupts splicing);

FIGS. 5A-5F are schematic diagrams illustrating the effect of internal homology according to some embodiments of the present disclosure (FIG. 5A: internal homologous sequence of the original Anabaena Exon; FIG. 5B: homology arm of 19 nt in length in the study sequence; FIG. 5C: effect of removing the 19 nt; FIGS. 5D-5F: effect of homologous sequences with different GC ratios and lengths on circularization efficiency);

FIGS. 6A-6B are schematic diagrams illustrating the overall design principle of the present disclosure;

FIGS. 7A-7E are schematic diagrams illustrating the design and results of circularizing IRES+Fluc according to some embodiments of the present disclosure (FIG. 7A: circular RNA containing only “IRES+Fluc”; FIG. 7B: splice site settings; FIG. 7C: circularization efficiency; FIG. 7D: translation efficiency; and FIG. 7E: in vitro reverse transcription PCR sequencing);

FIGS. 8A-8D are schematic diagrams illustrating the design and results of circularizing IRES+eGFP according to some embodiments of the present disclosure (FIG. 8A: settings of splice sites and internal homologous sequence; FIG. 8B: circularization efficiency; FIG. 8c: in vitro reverse transcription PCR sequencing; and FIG. 8D: translation efficiency);

FIGS. 9A-9G are schematic diagrams illustrating the effects of UTRs on circularization efficiency and translation efficiency according to some embodiments of the present disclosure (FIG. 9A: 5′ UTR screening; FIG. 9B: 3′ UTR screening; FIGS. 9C-9D: polyA sequence optimization; FIGS. 9E-9G: “IRES+ORF+UTR” scar-free circularization system);

FIG. 10 is a schematic diagram illustrating a position for inserting CVB3 IRES according to some embodiments of the present disclosure;

FIGS. 11A-11C are electropherograms of an IRES enhancer element according to some embodiments of the present disclosure;

FIGS. 12A-12J are schematic diagrams illustrating transfection effect of an IRES sequence inserted with an IRES enhancer element according to Example 4 of the present disclosure;

FIG. 13 is a schematic diagram illustrating a process for producing circular RNA according to some embodiments of the present disclosure;

FIG. 14 is a gel electropherogram illustrating a Gaussia luciferase circular RNA (Gluc-cRNA) production process according to some embodiments of the present disclosure;

FIG. 15 is a schematic diagram illustrating a first step NHS affinity purification of Gluc-cRNA according to some embodiments of the present disclosure;

FIG. 16 is a gel electropherogram illustrating affinity purification sampling of Gluc-cRNA according to some embodiments of the present disclosure (loading amount: 12.2 mg; total cRNA recovery: 10.23 mg; cRNA recovery rate: 83.85%; and purity: 88.2%);

FIG. 17 is a schematic diagram illustrating Gluc-cRNA purification via Core400 size exclusion chromatography according to some embodiments of the present disclosure;

FIG. 18 is an Agilent 5200 capillary electropherogram illustrating a two-step purification process of Gluc-cRNA according to some embodiments of the present disclosure (loading amount: 9.4 mg; total cRNA recovery: 8.01 mg; cRNA recovery rate: 85.21%; and purity: 98.9%).

### DETAILED DESCRIPTION

To address the current technical challenges in circular RNA (circRNA) research, the ribozyme from Anabaena is used as the research object, and the internal guide sequence (IG sequence) within the ribozyme is modified to recognize a desired circularization fragment, thereby achieving circRNA synthesis without exogenous sequence incorporation. Meanwhile, the present disclosure screens a plurality of UTR sequences. The screened UTR sequences have an enhancing effect on the translation of circRNA. The optimal combination improves the translation efficiency by 5.7-fold. Meanwhile, the IG sequence in the present disclosure also recognizes a UTR sequence, which enables the RNA circularization system to be a universal RNA circularization system. The universal RNA circularization system can screen and modify IRES sequences, such that for different circularization sequences, no other design is required.

Through research on the circularization mechanism of the PIE method, the present disclosure discovers that the occurrence of a circularization process is required to satisfy the following principles: 1) the 3 nucleotides of the IG sequence are required to satisfy the feature of “GNN”; 2) the IG sequence is required to ensure complementary base pairing or GU wobble pairing with the 3 nucleotides of the 5′ splice site; 3) the 3′ splice site should not have significant homology with the 5′ splice site and its adjacent nucleotide sequences, as such homology is not conducive to splicing and can greatly reduce circularization efficiency; and 4) at least 3 nucleotides adjacent to the 5′ splice site are required to have homology with at least 3 nucleotides adjacent to the 3′ splice site. In addition, the scar-free RNA circularization method described in the present disclosure, by modifying the IG sequence to recognize a UTR sequence, retains the advantage of high circularization efficiency. Moreover, due to recognition of the spacer sequence, the method is universal for different sequences to be circularized and requires no additional design.

Secondly, the present disclosure screens 4 IRES enhancer elements, and then explores the effect of IRES enhancer elements with different sequences with equivalent enhancing effects on the translation efficiency of circRNA. These 4 IRES enhancer elements all have a ribosome-recruiting function, thereby affecting the translation of circRNA. The 4 screened IRES enhancer element sequences are respectively cloned into a DNA template, the composition of which is the same as described above, for screening an optimal sequence to obtain a gene sequence with high protein expression. Finally, based on the optimal position of the IRES enhancer element and on screening the IRES enhancer element, the present disclosure performs a series of repeat sequence designs for the IRES enhancer element to obtain sequences that enable the IRES enhancer element to achieve its function. Among the screened sequences, IRES-1-A1-X2 improves by about 2.3-fold compared to an original sequence.

### TECHNICAL TERMS

When used in conjunction with the term “comprising” in the claims and/or the present disclosure, the words “a” or “an” may refer to “one”, but may also refer to “one or more” and “at least one”.

As used in the claims and the present disclosure, the terms “comprising”, “having”, “including”, or “containing” refer to inclusive or open-ended, and do not exclude additional, unrecited elements or method steps.

Throughout the present disclosure, the term “about” indicates that: a value includes the standard deviation of an error of a device or a method used to determine the value.

Although the present disclosure supports a definition of the term “or” as being merely an alternative and “and/or”, unless it is explicitly indicated as being merely an alternative or that the alternatives are mutually exclusive, the term “or” in the claims refers to “and/or”.

The terms “polypeptide”, “peptide”, and “protein” are used interchangeably in the present disclosure and refer to amino acid polymers of any length. The polymer may be linear or branched, it may comprise modified amino acids, and it may be interrupted by non-amino acid spacers and linkers.

In embodiments of the present disclosure, the nucleic acid or peptide sequence of the present disclosure comprises sequences with greater than 90% homology or sequence identity, preferably greater than 95%, more preferably 96%, 97%, 98%, or 99% homology or sequence identity. Methods for determining sequence homology or identity, which are well known to those of ordinary skill in the art, include, but are not limited to: Computational Molecular Biology, edited by Lesk, A. M., Oxford University Press, New York, 1988; Biocomputing: Informatics and Genome Projects, edited by Smith, D. W., Academic Press, New York, 1993; Computer Analysis of Sequence Data, Part I, edited by Griffin, A. M. and Griffin, H. G., Humana Press, New Jersey, 1994; Sequence Analysis in Molecular Biology, von Heinje, G., Academic Press, 1987; and Sequence Analysis Primer, edited by Gribskov, M. and Devereux, J., M Stockton Press, New York, 1991; and Carillo, H. and Lipman, D., SIAM J. Applied Math., 48:1073 (1988). A preferred method for determining identity is to obtain a maximal match between the tested sequences. Methods for determining identity are compiled in publicly available computer programs. Preferred computer program methods for determining identity between two sequences include, but are not limited to: the GCG package (Devereux, J., et al., 1984), BLASTP, BLASTN, and FASTA (Altschul, S. E, et al., 1990). The BLASTX program is publicly available from National Center for Biotechnology Information (NCBI) and other sources (BLAST Manual, Altschul, S., et al., NCBI NLM NIH Bethesda, Md. 20894; Altschul, S., et al., 1990). The well-known Smith-Waterman algorithm may also be used to determine identity.

The present disclosure provides a recombinant nucleic acid molecule for preparing circRNA, wherein in a 5′ to 3′ direction, the recombinant nucleic acid molecule comprises elements operably connected in sequence, the elements comprising an optional 5′ homology arm, a 3′ half-intron fragment, a circularization fragment, a 5′ half-intron fragment, and an optional 3′ homology arm; wherein a 5′ of the circularization fragment comprises a 3′ circularization recognition segment, and a 3′ of the circularization fragment comprises a 5′ circularization recognition segment; the 5′ half-intron fragment and the 3′ half-intron fragment are derived from a group I intron, and the group I intron is derived from Anabaena, specifically, Anabaena tRNA^{leu}; the 5′ half-intron fragment and the 3′ half-intron fragment form an intron sequence in the 5′ to 3′ direction; a nucleotide sequence of the 5′ half-intron fragment comprises a part of the intron sequence proximal to the 5′, a nucleotide sequence of the 3′ half-intron fragment comprises a remaining part of the intron sequence proximal to the 3′, and the 5′ half-intron fragment comprises an IG sequence of the intron sequence; wherein (a) the IG sequence of the intron sequence is 3 nt of 5′-GNN-3′, and the 5′ circularization recognition segment is 3 nt of 5′-NNY-3′, wherein Y is C or T/U; N is any nucleotide, and the IG sequence and bases corresponding to the 5′ circularization recognition segment have strict complementary base pairing or GU wobble pairing; (b) a 5′ of the circularization fragment comprises at least 2 nt of the 3′ circularization recognition segment, and the 3′ circularization recognition segment does not have significant homology with the 5′ circularization recognition segment and adjacent nucleotide sequences; and (c) an adjacent nucleotide sequence of the 5′ circularization recognition segment and an adjacent nucleotide sequence of the 3′ circularization recognition segment comprise at least 3 nt of an internal homologous sequence, wherein the significant homology refers to complementary base pairing between two or more nucleotides.

The recombinant nucleic acid molecule refers to a polynucleotide having sequences that are not linked together in nature. The polynucleotide may be contained in a suitable vector, and the vector may be used to transform a suitable host cell. The polynucleotide may be expressed in a recombinant host cell to produce, for example, a “recombinant polypeptide”, a “recombinant protein”, a “fusion protein”; or an RNA molecule may be obtained through in vivo or in vitro reverse transcription.

The host cell refers to a cell into which an exogenous polynucleotide has been introduced. In some embodiments, the host cell may include progeny of the cell into which the exogenous polynucleotide has been introduced. The host cell includes a ‘transformant’ and a ‘transformed cell’, which includes a primary transformed cell and progeny derived therefrom. The host cell may be any type of cell system that can be used to produce an antibody molecule of the present disclosure. The host cell may include a eukaryotic cell and a prokaryotic cell. For example, the eukaryotic cell may include a mammalian cell, an insect cell, and a yeast cell; and the prokaryotic cell may include an Escherichia coli (E. coli) cell. The host cell may include a cultured cell and may also include a cell within a transgenic animal, a transgenic plant, or a cultured plant tissue or animal tissue.

The recombinant host cell covers a host cell that, after being introduced with a recombinant nucleic acid molecule, a recombinant expression vector, or a circRNA, is different from a parental cell. The recombinant host cell is specifically obtained through transformation. The host cell of the present disclosure may be a prokaryotic cell or a eukaryotic cell, as long as it is a cell that can be introduced with a recombinant nucleic acid molecule, a recombinant expression vector, or a circRNA, or the like of the present disclosure.

The homology arm may also be referred to as a homologous sequence. The homology arm includes a 5′ homology arm located at a 5′ of a recombinant nucleic acid molecule, and a 3′ homology arm located at a 3′ of the recombinant nucleic acid molecule. The nucleic acid sequence of the 5′ homology arm is complementary to the nucleotide sequence of the 3′ homology arm.

The group I ribozyme refers to a “group I intron”. In some embodiments, the group I ribozyme or the group I intron has a self-splicing system that performs self-splicing and circularization in the presence of Guanosine Triphosphate (GTP) and Mg²⁺. The group I intron is preferably a group I intron from Anabaena, preferably derived from Anabaena tRNA^{leu}

In some embodiments, a group I intron is a large class of ribozymes that can undergo a self-splicing reaction and is generally widely present in a plurality of species. The group I intron mainly participates in catalyzing the excision of precursors of mRNA, transfer RNA (tRNA), and ribosomal RNA (rRNA).

In some embodiments, a group I intron may be divided into 10 domains according to a secondary structure and are respectively named P1 to P10. A catalytic center of the group I intron is located at a junction of P4-P6 (P4, P5, P6) and P3-P9 (P3, P7, P8, P9) domains, and P1 and P10 are respectively a 5′ splice site and a 3′ splice site. Other sequences surrounding the conserved catalytic center are responsible for maintaining the stability and proper folding of the ribozyme. A core secondary structure of the group I intron includes a pairing region (P) and a corresponding loop region (L) and splicing of the group I intron is performed through two successive transesterification reactions.

In some embodiments, an exogenous guanosine or guanosine nucleotide (G) first docks at an active G-binding site located at P7. A 3′-OH of the exogenous guanosine or G aligns and attacks a phosphodiester bond at a 5′ splice site located at P1, breaking the phosphodiester bond there, thereby generating a free 3′-OH group located at an upstream exon, and the G-binding site of the exogenous guanosine is connected to a 5′ of the intron. A terminal G (omega G) of the intron exchanges the exogenous G and occupies the G-binding site, organizing a second transesterification reaction: a 3′-OH group of an upstream exon of P1 aligns and attacks a 3′ splice site P10, causing adjacent upstream and downstream exons to ligate (circularize) and release of the catalytic intron.

In some embodiments, an intron fragment I and an intron fragment II, or a 5′ half-intron fragment and a 3′ half-intron fragment, have the same meaning, and are both derived from a group I intron, and respectively comprise a part of a sequence forming the group I intron proximal to a 5′ direction, and a part of the sequence forming the group I intron proximal to a 3′ direction.

In some embodiments, an internal split site of the group I intron may be determined by those of ordinary skill in the art. For example, those skilled in the art can refer to the following references to determine the internal split site of the group I intron: Puttaraju M, et al., (1992). group I permuted intron-exon (PIE) sequences self-splice to produce circular exons; Puttaraju M, et al., (1996). Circular ribozymes generated in E. coli using group I self-splicing PIE sequences.

Merely by way of example, for a group I intron, particularly for an Anabaena group I intron, it may generally be cleaved at a specific site within a P6 region of the group I intron to form a PIE system.

In some embodiments, a 3′ part of a natural group I intron (e.g., an Anabaena group I intron) is a part from a specific site within a P6 region of the natural group I intron to the 3′. In some embodiments, a 5′ part of a natural group I intron is a part from a specific site within a P6 region of the natural group I intron to the 5′. For a group I intron, particularly for an Anabaena group I intron, a splice site may also be located within a P2, P5, P8, or P9 region thereof to form a PIE system, as shown in WO 2021236855 A1, and the disclosure is incorporated herein by reference. Merely by way of example, the group I intron may also comprise various truncated bodies or other mutants having self-splicing activity (e.g., corresponding variants disclosed in WO 2020/237227 A1 (Tables 4 and 5), WO 2023046153 A1, or CN 115997018 A), and these disclosures are incorporated herein by reference.

In some embodiments, a self-splicing intron is the group I intron from cyanobacterium Anabaena. For example, the group I intron from the pre-tRNA-Leu gene of cyanobacterium Anabaena. Accordingly, in some embodiments, the 3′ self-splicing intron fragment (the 3′ group I intron fragment) and the 5′ self-splicing intron fragment (the 5′ group I intron fragment) are derived from the group I intron of cyanobacterium Anabaena, e.g., the group I intron from the pre-tRNA-Leu gene of cyanobacterium Anabaena.

In some embodiments, the natural group I intron from the pre-tRNA-Leu gene of cyanobacterium Anabaena comprises the nucleotide sequence set forth in SEQ ID NO: 43. The P6 region corresponds to the positions 98-157 of SEQ ID NO: 43. The splice site may be any position between positions 122-138 of SEQ ID NO: 43.

In some embodiments, a 3′ self-splicing intron fragment (the 3′ group I intron fragment) is derived from the group I intron of the pre-tRNA-Leu gene of cyanobacterium Anabaena, and comprises the nucleotide sequence of SEQ ID NO: 44 or a nucleotide sequence having at least 75% identity to SEQ ID NO: 44. For example, the 3′ self-splicing intron fragment comprises a nucleotide sequence having at least 80%, at least 85%, at least 90%, at least 95%, or 100% identity to SEQ ID NO: 44, or consists of SEQ ID NO: 44. In some embodiments, the 5′ self-splicing intron fragment (the 5′ group I intron fragment) is derived from the group I intron of the pre-tRNA-Leu gene of cyanobacterium Anabaena, and comprises the nucleotide sequence of SEQ ID NO: 45 or a nucleotide sequence having at least 75% identity to SEQ ID NO:45. For example, the 5′ self-splicing intron fragment comprises a nucleotide sequence having at least 80%, at least 85%, at least 90%, at least 95%, or 100% identity to SEQ ID NO: 45, or consists of SEQ ID NO: 45.

The IG sequence generally refers to a nucleotide sequence in the group I intron that is complementary to a corresponding exon sequence via Watson-Crick pairing or wobble pairing. In some embodiments, the IG sequence is generally located in a P1 stem of the group I intron, and the original IG sequence of the pre-tRNA-Leu gene is ‘GAG’.

The circularization fragment refers to an RNA molecule fragment that is eventually circularized or a corresponding DNA fragment thereof. The circularization fragment comprises a translation initiation element, a target polypeptide coding region or a non-coding region, and other optional regulatory and insertion elements (e.g., 5′ UTR or 3′ UTR), and also comprises a spacer fragment (i.e., a spacer sequence) between the intron fragments.

In some embodiments, the circularization fragment comprises a 5′ part of a target polypeptide coding region or a non-coding region, a translation initiation element, and a 3′ part of the target polypeptide coding region or the non-coding region. It is to be understood that the 5′ circularization recognition segment and the 3′ circularization recognition segment divide the target polypeptide coding region or the non-coding region into two parts, and after intron splicing, the two parts are re-ligated into a complete target polypeptide coding region or a non-coding region.

In some embodiments, the circularization fragment comprises a 5′ part of a translation initiation element, a target polypeptide coding region or a non-coding region, and a 3′ part of the translation initiation element. It is to be understood that the 5′ circularization recognition segment and the 3′ circularization recognition segment divide the translation initiation element into two parts, and after intron splicing, the 5′ part of the translation initiation element and the 3′ part of the translation initiation element are ligated into a complete translation initiation element region.

The 5′ circularization recognition segment is derived from an exon sequence (Exon 1, E1) linked to a 5′ of the group I intron, and the 3′ circularization recognition segment is derived from an exon sequence (Exon 2, E2) linked to a 3′ of the group I intron. In some embodiments, the IG sequence is configured to recognize the 5′ circularization recognition segment via Watson-Crick pairing or wobble pairing.

In some embodiments, E1 is an adjacent exon sequence adjacent to a 5′ splice site, wherein the adjacent exon sequence has a length of at least one nucleotide (e.g., the length of the adjacent exon sequence is at least 5 nucleotides, at least 10 nucleotides, at least 15 nucleotides, at least 20 nucleotides, at least 25 nucleotides, or at least 50 nucleotides); and E2 is an adjacent exon sequence adjacent to a 3′ splice site, wherein the adjacent exon sequence has a length of at least one nucleotide (e.g., the length of the adjacent exon sequence is at least 5 nucleotides, at least 10 nucleotides, at least 15 nucleotides, at least 20 nucleotides, at least 25 nucleotides, or at least 50 nucleotides). Merely by way of example, an optional E1 fragment of the group I intron from the pre-tRNA-Leu gene is CUU or CUC; and an optional E2 fragment is AAAA, AA, UUUU, CAAA, or GAAA.

It should be noted that E1 and E2 (including optimized E1 and E2) are retained in a circularization sequence to form a 5′-E1-E2-3′ “scar”, also referred to as a residual circularization sequence. In the present disclosure, due to the modification of the IG sequence, a selection of the 5′ circularization recognition segment is expanded, and a corresponding 5′ circularization recognition segment may be set in a translation initiation element, a coding region, or a spacer sequence. Similarly, the present disclosure also optimizes a number of bases and a base type of the 3′ circularization recognition segment, thereby expanding the selectability of the 3′ circularization recognition segment.

In the present disclosure, the 3′ circularization recognition segment does not have significant homology with the 5′ circularization recognition segment and adjacent nucleotide sequences thereof, thereby avoiding reducing circularization efficiency, and the significant homology refers to complementary base pairing between two or more (e.g., three) nucleotides.

The internal homologous sequence refers to a reverse complementary sequence existing between the nucleotide sequences adjacent to the 5′ circularization recognition segment and the 3′ circularization recognition segment in the circularization fragment. The internal homology refers to a reverse complementary relationship existing between the nucleotide sequences adjacent to the 5′ circularization recognition segment and the 3′ circularization recognition segment in the circularization fragment.

In some embodiments, the significant homology refers to complementary base pairing between two or more nucleotides.

Preferably, a length of the internal homologous sequence is 3 nucleotides (nt), 4 nt, or 5 nt; and/or a length of the 3′ circularization recognition segment is 2 nt.

In some embodiments, in E1 and E2, or in the nucleotides adjacent to the 5′ circularization recognition segment and the 3′ circularization recognition segment in an artificially inserted circularization fragment, there need to be a plurality of complementary fragments to form a “splice bubble”. For example, at least three nucleotides adjacent to the 5′ circularization recognition segment need to have homology with at least three nucleotides adjacent to the 3′ circularization recognition segment.

The splice bubble refers to a region between a homology arm and the internal homologous sequence, which comprises a splice ribozyme.

In some embodiments, the internal homology is provided by a spacer sequence.

The adjacent region or the adjacent sequence refers to a region or sequence of 1 to 20 bases that is linked to a described fragment, e.g., the 5′ circularization recognition segment or the 3′ circularization recognition segment and is located upstream or downstream thereof in the recombinant nucleic acid molecule. For example, the adjacent region or the adjacent sequence may be a region or sequence of 1 to 10 bases. In some embodiments, the adjacent sequence or the adjacent region of the 5′ circularization recognition segment or the 3′ circularization recognition segment refers to a sequence or region that is adjacent to the aforementioned circularization recognition segment and is located in the circularization fragment, i.e., it is retained with the circularization fragment in a final circRNA molecule.

In some embodiments, a 5′ of the circularization fragment has a 3′ circularization recognition segment-internal homologous sequence segment, and a 3′ thereof has an internal homologous sequence segment-5′ circularization recognition segment, with optionally 0, 1, 2, 3, 4, or 5 unpaired bases between the circularization recognition segment and the internal homologous sequence. Preferably, a length of the internal homologous sequence is 3 nt, 4 nt, or 5 nt; and/or a length of the 3′ circularization recognition segment is 2 nt.

In some embodiments, the circularization fragment includes the following elements operably linked in sequence: a first part of a target polypeptide coding region or a non-coding region comprising the 3′ circularization recognition segment, a translation initiation element, and a second part of a target polypeptide coding region or a non-coding region comprising the 5′ circularization recognition segment; wherein the target polypeptide coding region or the non-coding region is cleaved as follows: the second part containing the 5′ circularization recognition segment | the first part containing the 3′ circularization recognition segment.

The target polypeptide coding region or the non-coding region is not a naturally occurring nucleotide sequence. In some embodiments, the target polypeptide coding region encodes a natural or synthetic protein. In some embodiments, the coding or non-coding region may be a natural or synthetic sequence. In some embodiments, the coding region may encode a chimeric antigen receptor, an antibody, an immunoregulatory protein, and/or a transcription factor. In some embodiments, the non-coding region may encode a sequence that can alter cell behavior (e.g., lymphocyte behavior). In some embodiments, a non-coding sequence is antisense to a cellular RNA sequence.

In some embodiments, the circularization fragment comprises a 5′ UTR, a target polypeptide coding region, and a 3′ UTR, and the 5′ circularization recognition segment and the 3′ circularization recognition segment are respectively located in the 5′ UTR and the 3′ UTR.

The coding region refers to a gene sequence that is capable of transcribing mRNA and eventually translating into a target polypeptide or a protein.

In the present disclosure, the antibody is used in its broadest sense, referring to a protein comprising an antigen-binding site, and encompassing natural antibodies and artificial antibodies of various structures, including, but not limited to, a monoclonal antibody, a polyclonal antibody, a multi-specific antibody (e.g., a bispecific antibody), a single-chain antibody, an intact antibody, and an antibody fragment.

The present disclosure does not limit a type of a target polypeptide, and the target polypeptide may be a human protein or a non-human protein. Illustratively, the target polypeptide includes, but is not limited to, an antigen, an antibody, an antigen-binding fragment, a fluorescent protein, a protein having disease therapeutic activity, a protein having gene editing activity, or the like.

The antigen-binding fragment is a part or a segment of a complete or intact antibody that has fewer amino acid residues than the complete or intact antibody, which can bind to an antigen or compete with the complete antibody (i.e., the complete antibody from which the antigen-binding fragment is derived) for binding to the antigen. The antigen-binding fragment may be prepared by recombinant DNA technology, or by enzymatic or chemical cleavage of the complete antibody. The antigen-binding fragment includes, but is not limited to, Fragment variable (Fv), Fragment antigen-binding (Fab), Fragment antigen-binding prime (Fab′), Fragment antigen-binding prime with free sulfhydryl group (Fab′-SH), Dimeric fragment antigen-binding prime (F(ab′)₂); diabodies; linear antibodies; single chain antibodies (e.g., single-chain variable fragment (scFv)); single-domain antibodies; bivalent or bispecific antibodies or fragments thereof; camelid antibodies (heavy chain antibodies); and bispecific antibodies or multi-specific antibodies formed from antibody fragments.

In the present disclosure, the protein having disease-treating activity may include, but is not limited to, enzyme replacement proteins, supplemental proteins, protein vaccines, antigens (e.g., tumor antigens, viruses, bacteria), hormones, cytokines, antibodies, immunotherapies (e.g., cancer), cell reprogramming/trans differentiation factors, transcription factors, chimeric antigen receptors, transposases or nucleases, immune effectors (e.g., affecting susceptibility to immune responses/signals), regulated cell death effector proteins (e.g., inducers of apoptosis or necrosis), non-lytic inhibitors of tumors (e.g., oncoprotein inhibitors), epigenetic modifiers, epigenetic enzymes, transcription factors, DNA or protein modifying enzymes, DNA intercalators, efflux pump inhibitors, nuclear receptor activators or inhibitors, proteasome inhibitors, enzyme competitive inhibitors, protein synthesis effectors or inhibitors, nucleases, protein fragments or domains, ligands or receptors, and Clustered Regularly Interspaced Short Palindromic Repeats (CRISPR) systems or components thereof, or the like.

The translation initiation element may be any type of element that can initiate translation of a target polypeptide. In some embodiments, the translation initiation element comprises at least one of an IRES sequence, a 5′ UTR sequence, a Kozak sequence, a sequence containing an m⁶A modification (N⁶-methyladenosine modification), or a complementary sequence of ribosomal 18S rRNA. In some embodiments, the translation initiation element may also be any other type of cap-independent translation initiation element.

In some embodiments, the translation initiation element is an IRES sequence; the IRES sequence is derived from one of Taura syndrome virus, Triatoma virus, Theiler's encephalomyelitis virus, simian virus 40, Solenopsis invicta virus 1, Rhopalosiphum padi virus, reticuloendotheliosis virus, Poliovirus type 1 (Mahoney), Chrysodeixis includens virus, Kashmir bee virus, human rhinovirus 2, Homalodisca coagulata virus-1, human immunodeficiency virus type 1, Homalodisca coagulata virus-1, Himetobi P virus, hepatitis C virus, hepatitis A virus, GB hepatitis virus, foot-and-mouth disease virus, human enterovirus 71, equine rhinitis virus, Ectropis obliqua picorna-like virus, encephalomyocarditis virus (EMCV), Drosophila C virus, crucifer-infecting tobamovirus, cricket paralysis virus, bovine viral diarrhea virus 1, black queen cell virus, aphid lethal paralysis virus, avian encephalomyelitis virus, acute bee paralysis virus, hibiscus chlorotic ringspot virus, classical swine fever virus, human FGF2, human SFTPA1, human AMLl/RUNXl, Drosophila antennapedia, human AQP4, human AT1R, human BAG-1, human BCL2, human BiP, human c-IAPI, human c-myc, human eIF4G, mouse NDST4L, human LEF1, mouse HIF1α, human n-myc, mouse Gtx, human p27kip1, human PDGF2/c-sis, human p53, human Pim-1, mouse Rbm3, Drosophila reaper, canine Scamper, Drosophila Ubx, human UNR, mouse UtrA, human VEGF-A, human XIAP, salivirus, coxsackievirus, parechovirus, Drosophila hairless, Saccharomyces cerevisiae TFIID, Saccharomyces cerevisiae YAP1, human c-src, human FGF-1, simian picornavirus, turnip crinkle virus, aptamer of eIF4G, coxsackievirus B3 (CVB3), or coxsackievirus A (CVB1/2). Further preferably, the IRES sequence is an IRES sequence of Coxsackievirus B3 (CVB3); more preferably, the IRES sequence is an IRES sequence of Encephalomyocarditis virus (EMCV).

In some embodiments, the circularization fragment comprises the following elements operably linked in sequence: a first part of a translation initiation element comprising the 3′ circularization recognition segment, a first part of a target polypeptide coding region or a non-coding region, and a second part of the translation initiation element comprising the 5′ circularization recognition segment; and the translation initiation element is cleaved as follows: the second part containing the 5′ circularization recognition segment I the first part containing the 3′ circularization recognition segment.

In some embodiments, the circularization fragment comprises the following elements operably linked in sequence: a 5′ spacer sequence, an optional translation initiation element, a target polypeptide coding region or a non-coding region, and a 3′ spacer sequence; preferably, a total number of exogenously introduced bases in the 5′ spacer sequence and the 3′ spacer sequence being less than 10; preferably, inserting 3 nucleotides at a third or fourth nucleotide position of the 5′ spacer sequence as the internal homologous sequence, using two or three nucleotides carried by the 5′ spacer sequence as the 3′ circularization recognition segment, and inserting 6 nucleotides at a 3′ of the 3′ spacer sequence, wherein the 1st-3rd nucleotides are complementary to the nucleotides inserted in the 5′ spacer sequence, and the 4th-6th sequences are NNY, and NNY has strict complementary base pairing or GU wobble pairing with nucleotides corresponding to the IG sequence; more preferably, inserting 5 nucleotides at a 5′ of the 5′ spacer sequence, wherein the 1st-2nd nucleotides serve as the 3′ circularization recognition segment, the 3rd-5th nucleotides serve as the internal homologous sequence, using two or three nucleotides carried by the 5′ spacer sequence as the 3′ circularization recognition segment; inserting 6 nucleotides at a 3′ of the 3′ spacer sequence, wherein the 1st-3rd nucleotides are complementary to the nucleotides inserted in the 5′ spacer sequence, and the 4th-6th sequences are NNY

The spacer sequence refers to any continuous nucleotide sequence. For example, the spacer sequence may include 1) a nucleotide sequence predicted to avoid interference with proximal structures (e.g., from an IRES, a coding or non-coding region, or an intron), 2) a nucleotide sequence having at least 7 nucleotides (optionally no more than 100 nucleotides), 3) a nucleotide sequence located downstream and proximal to the 3′ intron fragment and/or upstream and proximal to the 5′ intron fragment, and/or 4) a nucleotide sequence comprising one or more of the following conditions: a) an unstructured region of at least 3 nt in length, b) a region predicted to form base pairing of at least 3 nt in length with a distal (i.e., non-adjacent) sequence, which includes another spacer sequence, and/or c) a structured region of at least 7 nt in length, the scope of which is limited to the spacer sequence.

In some embodiments, a length of the spacer sequence may be at least 10 nucleotides, at least 15 nucleotides, or at least 30 nucleotides. In some embodiments, the length of the spacer sequence is at least 7, 8, 9, 10, 11, 12, 13, 14, 15, 16, 17, 18, 19, 20, 25, or 30 nucleotides. In some embodiments, the length of the spacer sequence is no more than 100, 90, 80, 70, 60, 50, 45, 40, 35, or 30 nucleotides. In some embodiments, the length of the spacer sequence is 20 to 50 nucleotides. In some embodiments, the length of the spacer sequence is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides.

In some embodiments, the spacer sequence may be a polyA sequence, a polyA-C sequence, a polyC sequence, or a poly-U sequence, or may be specifically modified according to the IRES. The spacer sequence described in the present disclosure can have two functions: (1) facilitating circularization and (2) enhancing translation efficiency. More specifically, the spacer sequence described in the present disclosure is designed to have the following functions: 1) being inert to the folding of proximal intron and IRES structures; 2) adequately separating intron and IRES secondary structures; 3) carrying a splice site; 4) comprising a spacer sequence-spacer sequence complementary region to facilitate the formation of a ‘splice bubble’; and 5) enhancing translation efficiency.

Merely by way of example, the 5′ spacer sequence may be one of the following three types: the first type is a protein binding sequence, which can enhance translation efficiency by binding and recruiting proteins, wherein two sequences are 31A8CA and 41A8CA, respectively; the second type is an IRES enhancer sequence, wherein the IRES binds to 18S rRNA, eIF4G, and eIF4A; and the third type is a full-length UTR sequence.

Merely by way of example, the 5′ spacer sequence and the 3′ spacer sequence may be used together. For example, the 5′ spacer sequence and the 3′ spacer sequence may both be a combination structure of polyA+8CA, wherein a polyA segment may contain 10 to 100 A or 60 to 80 A; merely by way of example, the 5′ of the polyA segment may be 61 A and the 3′ may be 71 A.

In some embodiments, splice sites and internal homologous sequences are set in the 5′ spacer sequence and the 3′ spacer sequence, thereby forming a universal circularization system.

In some embodiments, the 5′ spacer sequence is selected from the following:

| | |
|---|---|
| 5′random | GGAGTGCTAGAGGATGACCTTAAATCAGGGACGATATTATAATTGACT |
| | AA (SEQ ID NO: 8) |
| 5′Apt- | ACTCACTATTTGTTTTCGCGCCCAGTTGCAAAAAGTGTCGTAATTGAC |
| eIF4G | TAA (SEQ ID NO: 9) |
| 5′Gtx56 | CCGGCGGCCGGCGGCCGGCGGCCGGCGGCCGGCGGCCGGCGGCCG |
| | GCGGCCGGCGG (SEQ ID NO: 10) |
| 5′ Gtx54 | CCGGCGGGTCCGGCGGGTCCGGCGGGTCCGGCGGGTCCGGCGGGTC |
| | CGGCGGGT (SEQ ID NO: 11) |
| 5′OR4F17 | TACTCCCTACTCCCTACTCCCTACTCCCTACTCCCTACTCCCTACTCCC |
| | TACTCCC (SEQ ID NO: 12) |
| 5′NeoUTR | CATTCTGTGGTCTGATCATCCTGTGGTTTCGTCGCCGCCATCCTCGTC |
| 1 | GCGACACGCTGTTTTCGGTTCTCGGCCCGACGAGCCATCGCCATCCT |
| | ACAGC (SEQ ID NO: 13) |
| 5′NeoUTR | CACTCGCGCTGCCATCACTCTTCCGCCGTCTTCGCCGCCATCCTCGGC |
| 2 | GCGACTCGCTTCTTTCGGTTCTACCAGGTAGAGTCCGCCGCCATCCTC |
| | CACC (SEQ ID NO: 14) |
| 5′NeoUTR | CTTGTCTCGCTCCGGGGAACGCTCGGAAACTCCCGGCCGCCGCCACC |
| 3 | CGCGTCTGTTCTGTTACACAAGGGAAGAAAAGCCGCTGCCGCACTCC |
| | GAGTGT (SEQ ID NO: 15) |
| 5′31A8CA | AAAAAAAAAAAAAAAAAAAAAAAAAAAAAAACCCCCCCCA (SEQ ID |
| | NO: 16) |
| 5′41A8CA | AAAAAAAAAAAAAAAAAAAAAAAAAAAAAAAAAAAAAAAAACCC |
| | CCCCCA (SEQ ID NO: 17) |
| 5′51A8CA | AAAAAAAAAAAAAAAAAAAAAAAAAAAAAAAAAAAAAAAAAAAA |
| | AAAAAAACCCCCCCCA (SEQ ID NO: 18) |
| 5′61A8CA | AAAAAAAAAAAAAAAAAAAAAAAAAAAAAAAAAAAAAAAAAAAA |
| | AAAAAAAAAAAAAAAAACCCCCCCCA (SEQ ID NO: 19) |
| 5′71A8CA | AAAAAAAAAAAAAAAAAAAAAAAAAAAAAAAAAAAAAAAAAAAA |
| | AAAAAAAAAAAAAAAAAAAAAAAAAAACCCCCCCCA |
| | (SEQ ID NO: 20) |
| 5′81A8CA | AAAAAAAAAAAAAAAAAAAAAAAAAAAAAAAAAAAAAAAAAAAA |
| | AAAAAAAAAAAAAAAAAAAAAAAAAAAAAAAAAAAAACCCCCCCC |
| | A (SEQ ID NO: 21) |

The 3′ spacer sequence is selected from the following:

| 3′ARE | TAATATTTATATATTTATATTTTTAAAATATTTATTTATTTATTTATTTA |
|---|---|
| | (SEQ ID NO: 23) |
| 3′CYBA | CCTCGCCCCGGACCTGCCCTCCCGCCAGGTGCACCCACCTGCAATAA |
| | ATGCAGCGAAGCCGGGA (SEQ ID NO: 24) |
| 3′DECR1 | GACCACTTTGGCCTTCATCTTGGTTACAGAAAAGGGAATAGAAATGA |
| | AACAAATTATCTCTCATCTTTTGACTATTTCAAGTCTAATAAATTCTTA |
| | ATTAAC (SEQ ID NO: 25) |
| 3′GADP | GACCCCTGGACCACCAGCCCCAGCAAGAGCACAAGAGGAAGAGAG |
| H | AGACCCTCACTGCTGGGGAGTCCCTGCCACACTCAGTCCCCCACCAC |
| | ACTGAATCTCCCCTCCTCACAGTTGCCATGTAGACCCCTTGAAGAGG |
| | GGAGGGGCCTAGGGAGCCGCACCTTGTCATGTACCATCAATAAAGTA |
| | CCCTGTGCTCAACCAGGAGCTCCC (SEQ ID NO: 26) |
| 3′ human | GCTGGAGCCTCGGTGGCCATGCTTCTTGCCCCTTGGGCCTCCCCCCA |
| α-globin | GCCCCTCCTCCCCTTCCTGCACCCGTACCCCCGTGGTCTTTGAATAAA |
| | GTCTGA (SEQ ID NO: 27) |
| 3′ human | GCTCGCTTTCTTGCTGTCCAATTTCTATTAAAGGTTCCTTTGTTCCCTA |
| β-globin | AGTCCAACTACTAAACTGGGGGATATTATGAAGGGCCTTGAGCATCTG |
| | GATTCTGCCT (SEQ ID NO: 28) |
| 3′OR4F1 | TACTCCCTACTCCCTACTCCCTACTCCCTACTCCCTACTCCCTACTCCC |
| 7 | TACTCCC (SEQ ID NO: 29) |
| 3′NeoUT | CACTCGCGCTGCCATCACTCTTCCGCCGTCTTCGCCGCCATCCTCGGC |
| R2 | GCGACTCGCTTCTTTCGGTTCTACCAGGTAGAGTCCGCCGCCATCCTC |
| | CACC (SEQ ID NO: 30) |
| 3′11A8C | AAAAAAAAAAACCCCCCCCA (SEQ ID NO: 31) |
| A | |
| 3′15A8C | AAAAAAAAAAAAAAACCCCCCCCA (SEQ ID NO: 32) |
| A | |
| 3′21A8C | AAAAAAAAAAAAAAAAAAAAACCCCCCCCA (SEQ ID NO: 33) |
| A | |
| 3′31A8C | AAAAAAAAAAAAAAAAAAAAAAAAAAAAAAACCCCCCCCA (SEQ ID |
| A | NO: 34) |
| 3′41A8C | AAAAAAAAAAAAAAAAAAAAAAAAAAAAAAAAAAAAAAAAACCC |
| A | CCCCCA (SEQ ID NO: 35) |
| 3′71A8C | AAAAAAAAAAAAAAAAAAAAAAAAAAAAAAAAAAAAAAAAAAAA |
| A | AAAAAAAAAAAAAAAAAAAAAAAAAAACCCCCCCCA (SEQ ID NO: |
| | 36) |

Preferably, the 5′ spacer sequence and the 3′ spacer sequence are used individually or in combination; preferably, the 5′ spacer sequence and the 3′ spacer sequence are both a combination structure of polyA+8CA, preferably, the polyA segment contains 10 to 100 A, more preferably, the polyA segment contains 60 to 80 A, most preferably, the 5′ of the polyA segment has 61 A and the 3′ of the polyA segment has 71 A; most preferably, the 5′ spacer sequence and the 3′ spacer sequence are a combination of 5′61A8CA and 3′71A8CA

In some embodiments, the translation initiation element is a CVB3 IRES sequence into which an IRES enhancer element is inserted: the IRES enhancer element is inserted between domain I and domain II of the IRES sequence (designated as IRES-1), at a stem-loop structure of the domain II of the IRES sequence (designated as IRES-2), at a distal loop of domain IV of the IRES sequence (designated as IRES-3), or at a proximal loop of domain IV of the IRES sequence (designated as IRES-4).

The IRES enhancer element is a sequence set forth below:

| Serial No. | IRES sequence |
|---|---|
| A1 | CCGGCGGGT |
| A2 | GTTTCATTTTATTCCTA |
| | TAC (SEQ ID NO: 46) |
| A3 | CAATTGAGAGATCGTTAC |
| | CATATAGCTA (SEQ ID NO: 47) |
| A4 | ATTTTATTCCTA (SEQ ID NO: 48) |

or repeat sequences of the above sequence:

| Serial | IRES Repeat Sequence |
|---|---|
| A1-X2 | CCGGCGGGTCCGGCGGGT |
| | (SEQ ID NO: 49) |
| A1-X5 | CCGGCGGGTCCGGCGGGTCCGG |
| | CGGGTCCGGCGGGTCCGGCGGGT |
| | (SEQ ID NO: 50) |
| A2-X2 | GTTTCATTTTATTCCTATACGTT |
| | TCATTTTATTCCTATAC |
| | (SEQ ID NO: 51) |

The IRES enhancer element refers to a short nucleotide sequence inserted into a specific domain of the CVB3 IRES sequence.

In some embodiments, the translation initiation element comprises at least one of an IRES sequence, a 5′ UTR sequence, a Kozak sequence, a sequence containing an m⁶A modification, or a complementary sequence to ribosomal 18S rRNA.

In some embodiments, the target polypeptide coding region or the non-coding region is a protein coding region encoding a human protein or a non-human protein; the protein coding region encodes an antibody; the human protein or the non-human protein is selected from a group consisting of hFIX, SP-B, VEGF-A, human methylmalonyl-CoA mutase (hMUT), CFTR, a cancer autoantigen, and a gene editing enzyme, wherein the gene editing enzyme includes Cpf1, a zinc finger nuclease (ZFN), and a transcription activator-like effector nuclease (TALEN); a protein is a protein for therapeutic use; the antibody is a human anti-HIV antibody; the antibody is a bispecific antibody; the bispecific antibody targets CD3 and CLDN6, or targets CD19 and CD22; the protein is a protein for diagnostic use; the protein coding region encodes Gaussia luciferase (Gluc), firefly luciferase (Fluc), enhanced green fluorescent protein (eGFP), human erythropoietin (hEPO), or Cas9 endonuclease; wherein the protein coding region comprises at least two coding regions, and any two adjacent coding regions are connected by a linker; and the linker is a polynucleotide encoding a 2A peptide.

In some embodiments, the recombinant nucleic acid molecule further comprises an insertion element, wherein the insertion element is located upstream of a translation initiation element; the insertion element is selected from at least one of a transcriptional regulatory element, a translational regulatory element, or a purification element; and the insertion element comprises at least one of a UTR sequence, a polyA sequence, an aptamer sequence, a riboswitch sequence, or a sequence that binds a transcriptional regulatory factor.

In some embodiments, the recombinant nucleic acid molecule comprises an insertion element, wherein the insertion element may regulate the transcription of the recombinant nucleic acid molecule, regulate the translation of the circRNA, facilitate specific expression of the circRNA among different tissues, or be used for purification of the circRNA, or the like. Merely by way of example, the insertion element is located between the coding element and the translation initiation element.

In some embodiments, the coding element in the recombinant nucleic acid molecule comprises at least one coding region. Merely by way of example, the coding element comprises 1, 2, 3, 4, 5, 10, 15, 20, or 25 coding regions. In some embodiments, the coding element comprises at least two coding regions, and each coding region independently encodes any type of target polypeptide.

In some embodiments, the coding element comprises two or more coding regions. The recombinant nucleic acid molecule comprises elements arranged in the following order in a 5′ to 3′ direction: an intron fragment II, a truncated fragment II of a translation initiation element, at least two coding regions, a truncated fragment I of the translation initiation element, and an intron fragment I. In some embodiments, the recombinant nucleic acid molecule is composed of the elements arranged in the above-mentioned order.

In some embodiments, the coding element further comprises a linker located between any two adjacent coding regions. By using the linker to separate the adjacent coding regions, the circRNA prepared by the recombinant nucleic acid molecule is allowed to achieve expression of two or more target polypeptides.

In some embodiments, the linker may be a polynucleotide encoding a 2A peptide, or another type of polynucleotide encoding a linking peptide configured for spacing target polypeptides. The 2A peptide is a short peptide derived from a virus (approximately 18-25 amino acids), and it is commonly referred to as “self-cleaving” peptides, which can cause a single transcript to produce multiple proteins. Merely by way of example, the 2A peptide includes P2A, T2A, E2A, F2A, or the like.

In some embodiments, the coding element comprises at least two coding regions, wherein the translation initiation element is linked between any two adjacent coding regions. Merely by way of example, the coding element comprises 1, 2, 3, 4, 5, 10, 15, 20, or 25 coding regions, or the like. Furthermore, within the coding element, the translation initiation element is arranged between any two adjacent coding regions. By the above-described approach, the translation initiation element may be linked upstream of each coding region in the circRNA prepared in vitro by the recombinant nucleic acid molecule, thereby achieving expression of two or more target polypeptides.

In some embodiments, the coding element comprises two or more coding regions. The recombinant nucleic acid molecule comprises elements arranged in the following order in a 5′ to 3′ direction: an intron fragment II, a truncated fragment II of a translation initiation element, at least two coding regions, a truncated fragment I of the translation initiation element, and an intron fragment I. The translation initiation element is linked between any two adjacent coding regions. In some embodiments, the recombinant nucleic acid molecule is composed of the elements arranged in the above-mentioned order.

In some embodiments, the translation initiation element may be any type of element that can initiate translation of a target polypeptide. In some embodiments, the translation initiation element comprises at least one of an IRES sequence, a 5′ UTR sequence, a Kozak sequence, a sequence containing an N6-methyladenosine (m6A) modification, or a complementary sequence to ribosomal 18S rRNA. In some embodiments, the translation initiation element may also be any other type of cap-independent translation initiation element.

In some embodiments, each coding region contained in the coding element independently encodes any type of target polypeptide. The target polypeptides encoded by any two coding regions may be the same or different.

In the circRNA prepared by the above-described recombinant nucleic acid molecule, the translation initiation element is correspondingly linked to the 5′ of each coding region, and by linking the plurality of coding regions in series via the plurality of translation initiation elements, expression of at least two target polypeptides is achieved.

In some embodiments, in a polyN sequence, N is selected from at least one of A, T, G, or C.

In some embodiments, the translational regulatory element comprises a UTR sequence, wherein the UTR sequence may be used to regulate the stability and immunogenicity of the circRNA, and efficiency of the circRNA in expressing a target polypeptide. The present disclosure does not specifically limit the UTR sequence, which may be selected from any type of sequence in the art that has properties such as regulating transcription, translation, intracellular stability, and immunogenicity of the circRNA, or the like. Furthermore, the UTR sequence is not limited to a 5′ UTR sequence or a 3′ UTR sequence.

In some embodiments, the untranslated region (UTR) sequence comprises one or more microRNA (miRNA) recognition sequences, for example, 1, 2, 3, 4, 5, 6, or 7 miRNA recognition sequences. By incorporating one or more miRNA recognition sequences, specific expression of the circRNA among different tissues and cells can be facilitated, thereby achieving targeted delivery of the circRNA molecule.

In some embodiments, the translational regulatory element comprises a polyN sequence, wherein the N may be at least one of A, T, G, or C. By adding the translational regulatory element comprising the polyN sequence, the efficiency of circRNA expressing a target polypeptide, immunogenicity, and stability can be improved, or it can be used to facilitate the purification of the circRNA. The present disclosure does not specifically limit the length of the polyN sequence, the type of N in the polyN sequence, or a composition manner, as long as it can facilitate improving the performance of the circRNA. Merely by way of example, the polyN sequence may be a polyA sequence, a polyAC sequence, or the like.

In some embodiments, the translational regulatory element comprises a riboswitch sequence. The riboswitch sequence refers to a type of untranslated region sequences that possess regulatory functions for transcription and translation of RNA. In the present disclosure, the riboswitch sequence can affect expression of the circRNA, including but not limited to transcriptional termination, translational initiation inhibition, mRNA self-cleavage, and changes in splice pathways in eukaryotes. Furthermore, the riboswitch sequence can also control expression of the circRNA by binding or removal of a trigger molecule. Merely by way of example, the riboswitch sequence may be a cobalamin riboswitch (also referred to as a B12-element), an FMN riboswitch (also referred to as an RFN element), a glmS riboswitch, a SAM riboswitch, a SAH riboswitch, a tetrahydrofolate riboswitch, a Moco riboswitch, or the like. The present disclosure does not restrictively limit the type and sequence of the riboswitch sequence, as long as it can achieve regulation of transcription and translation levels of a target polypeptide expressed by the circRNA.

In some embodiments, the translational regulatory element comprises an aptamer sequence. In the present disclosure, the aptamer sequence may be configured to regulate transcription and translation of the circRNA or configured to facilitate in vitro purification and preparation of the circRNA.

Expression includes any steps involved in polypeptide production, including but not limited to: transcription, post-transcriptional modification, translation, post-translational modification, and secretion.

In some embodiments, the target polypeptide coding region or the non-coding region is a protein coding region encoding a human protein or a non-human protein; and the recombinant nucleic acid molecule comprises homology arms, wherein each of the homology arms has a length of about 5 to 50 nucleotides, wherein each of the homology arms has a length of about 9 to 19 nucleotides.

The present disclosure provides a recombinant expression vector, and the recombinant expression vector comprises the recombinant nucleic acid molecule.

The recombinant expression vector refers to a DNA structure configured to express a polynucleotide encoding a desired polypeptide. The recombinant expression vector may include an expression cassette. For example, the expression cassette comprises i) a collection of genetic elements that possess regulatory effects on gene expression (e.g., a promoter and an enhancer); ii) a structure or coding sequence transcribed into mRNA and translated into a protein; and iii) a transcriptional subunit of appropriate transcription and translation initiation and termination sequences. The recombinant expression vector may be constructed in any suitable manner. The nature of the recombinant expression vector may include plasmids, viruses, phages, and transposons. Possible vectors for use in the present disclosure include but are not limited to chromosomal, non-chromosomal, and synthetic DNA sequences, e.g., viral plasmids, bacterial plasmids, phage DNA, yeast plasmids, and vectors derived from combinations of plasmids and phage DNA, DNA from viruses such as lentiviruses, retroviruses, vaccinia, adenoviruses, fowlpox, baculoviruses, Simian virus 40 (SV40), and pseudorabies.

The vector refers to a segment of DNA that is synthesized (e.g., using PCR) or extracted from a virus, a plasmid, or a cell of a higher organism, into which an exogenous DNA fragment may be or has been inserted for cloning and/or expression purposes. In some embodiments, the vector may be stably maintained in an organism. The vector may comprise, for example, an origin of replication, a selectable marker, or a reporter gene, such as antibiotic resistance or GFP, and/or an MCS. The term includes linear DNA fragments (e.g., Polymerase Chain Reaction (PCR) products, linear plasmid fragments), plasmid vectors, viral vectors, cosmids, bacterial artificial chromosomes (BACs), yeast artificial chromosomes (YACs), or the like.

In some embodiments, the vector can allow for production of translatable and/or biologically active circRNA within a eukaryotic cell.

In some embodiments, the recombinant nucleic acid molecule may exist as a part of the recombinant expression vector for preparing circRNA, and during an in vitro transcription and circularization process, a circRNA expressing a target polypeptide may be prepared.

In some embodiments, the recombinant nucleic acid molecule may also exist as a circularization precursor RNA molecule obtained after linearization and transcription reactions of the recombinant expression vector, or a part thereof. That is, the recombinant nucleic acid molecule only needs to undergo a circularization reaction to obtain a circRNA expressing a target polypeptide.

In some embodiments, circular RNA or circRNA has the same meaning, both referring to a scar-free RNA circular molecule obtained by the method of the present disclosure.

The present disclosure provides a composition, comprising: the recombinant nucleic acid molecule, a recombinant expression vector comprising the recombinant nucleic acid molecule, or a circRNA prepared based on the recombinant nucleic acid molecule; and one or more pharmaceutically acceptable carriers. The pharmaceutically acceptable carrier comprises at least one of a lipid, a polymer, or a lipid-polymer complex.

In some embodiments, the circRNA products produced using the vector and/or method described in the present disclosure may be provided in the form of a composition (e.g., a pharmaceutical composition).

Accordingly, the present disclosure also relates to a composition, e.g., a composition comprising circRNA (circRNA products) and a pharmaceutically acceptable carrier. In one aspect, the present disclosure provides a pharmaceutical composition comprising an effective amount of the circRNA described in the present disclosure and a pharmaceutically acceptable excipient. The pharmaceutical composition of the present disclosure may comprise the circRNA described in the present disclosure, and one or more pharmaceutically or physiologically acceptable carriers, excipients, or diluents. In some embodiments, the pharmaceutical composition of the present disclosure may comprise circRNA expressing cells, e.g., a plurality of circRNA expressing cells as described in the present disclosure, in combination with one or more pharmaceutically or physiologically acceptable carriers, excipients, or diluents.

In some embodiments, the pharmaceutically acceptable carrier may be any component in a pharmaceutical composition other than an active ingredient non-toxic to a subject.

The pharmaceutically acceptable carrier may include but is not limited to a buffering agent, an excipient, a stabilizer, or a preservative. Examples of pharmaceutically acceptable carriers include physiologically compatible solvents, dispersion media, coatings, antibacterial and antifungal agents, isotonic agents, and absorption delaying agents, such as salts, buffers, carbohydrates, antioxidants, aqueous or non-aqueous carriers, preservatives, wetting agents, surfactants, or emulsifiers, or combinations thereof. The amount of the pharmaceutically acceptable carrier in the pharmaceutical composition may be determined experimentally based on the activity of the carrier and the desired properties of the formulation, such as stability and/or minimal oxidation.

In some embodiments, such a composition may comprise a buffer, such as acetic acid, citric acid, histidine, boric acid, formic acid, succinic acid, phosphoric acid, carbonic acid, malic acid, aspartic acid, Tris(hydroxymethyl)aminomethane buffer (Tris buffer), HEPPSO, HEPES, neutral buffered saline, phosphate buffered saline; carbohydrates, such as glucose, sucrose, mannose, dextran, or mannitol; proteins; polypeptides or amino acids, such as glycine; antioxidants; chelating agents, such as EDTA or glutathione; adjuvants (e.g., aluminum hydroxide); antibacterial and antifungal agents; and preservatives.

In some embodiments, the composition of the present disclosure may be formulated for administration in a plurality of manners, including parenteral and non-parenteral routes. In one embodiment, the composition may be formulated for infusion or intravenous administration. The composition disclosed in the present disclosure may be provided, for example, as a sterile liquid preparation, e.g., an isotonic aqueous solution, an emulsion, a suspension, a dispersion, or a viscous composition, which may be buffered to a desired pH. Formulations suitable for oral administration may include liquid solutions, capsules, sachets, tablets, lozenges, and pastilles, liquid suspensions, powders, and emulsions in a suitable liquid.

The present disclosure provides an IRES enhancer element for insertion into a CVB3 IRES sequence to enhance a translation efficiency. The IRES enhancer element is inserted between domain I and domain II of an IRES sequence (designated IRES-1), at a stem-loop structure of domain II of the IRES sequence (designated IRES-2), at a distal loop of domain IV of the IRES sequence (designated IRES-3), or at a proximal loop of domain IV of the IRES sequence (designated IRES-4).

Unless otherwise defined or clearly indicated by context, all technical and scientific terms in the present disclosure have the same meaning as commonly understood by one of ordinary skill in the art to which the present disclosure belongs.

The present disclosure provides a universal scar-free RNA circularization method. The method includes, but is not limited to, a DNA construct for preparing circRNA, a recombinant expression vector comprising the DNA construct, a circularization precursor RNA molecule obtained by in vitro transcription using the recombinant expression vector, or the like.

The present disclosure first provides a recombinant nucleic acid molecule for preparing circRNA. Merely by way of example, the recombinant nucleic acid molecule may be the above-mentioned DNA construct for preparing circRNA or the circularization precursor RNA molecule.

As used in the present disclosure, the terms “complementary” or “hybridized” are used to refer to polynucleotides and oligonucleotides (which are interchangeable terms, referring to nucleotide sequences) related to base pairing rules. For example, the sequence “CAGT” is complementary to the sequence “GTCA”. The complementarity or hybridization may be “partial” or “total”. The ‘partial’ complementarity or hybridization refers to one or more nucleic acid bases being mismatched according to base pairing rules. The “total” or “complete” complementarity or hybridization between nucleic acids refers to each nucleic acid base being matched to another base according to base pairing rules. The degree of complementarity or hybridization between nucleic acid strands has a significant impact on the hybridization efficiency and strength between the nucleic acid strands. This is particularly important in amplification reactions and detection methods that depend on binding between nucleic acids.

As used in the present disclosure, the terms “individual”, “patient”, or “subject” include mammals. The mammals include, but are not limited to, domestic animals (e.g., cattle, sheep, cats, dogs, and horses), primates (e.g., humans and non-human primates such as monkeys), rabbits, and rodents (e.g., mice and rats).

As used in the present disclosure, the terms “transformation”, “transfection”, “transduction” have a meaning generally understood by a person of ordinary skill in the art, that is, a process of introducing exogenous DNA into a host. The methods of transformation, transfection, and transduction include any method of introducing a nucleic acid into a cell, and these methods include, but are not limited to, electroporation, calcium phosphate (CaPO₄) precipitation, calcium chloride (CaCl₂)) precipitation, microinjection, polyethylene glycol (PEG) method, DEAE-dextran method, cationic liposome method, and lithium acetate-Dimethyl sulfoxide (DMSO) method.

As used in the present disclosure, the term ‘treatment’ refers to exposing (e.g., administering) a subject to the circRNA, the circularization precursor RNA, the composition of the present disclosure, or the like, after suffering from a disease, thereby alleviating the symptoms of the disease as compared to when not exposed, which does not necessarily mean that the symptoms of the disease must be completely inhibited. The term “suffering from a disease” refers to a body having symptoms of a disease.

As used in the present disclosure, the term “effective amount” refers to an amount or dose of the recombinant nucleic acid molecule, the recombinant expression vector, the circularization precursor RNA, the circRNA, the vaccine, or the composition of the present disclosure, which, after being administered to a patient in a single or a plurality of doses, produces an anticipated effect in a patient in need of treatment or prevention. The effective amount may be readily determined by an attending physician of ordinary skill in the art by considering a plurality of factors, such as the species of the mammal; its size, age, and general health; the specific disease involved; the extent or severity of the disease; the response of the individual patient; the specific antibody administered; the mode of administration; the bioavailability characteristics of the administered formulation; the dosage regimen selected; and the use of any concomitant therapy.

As used in the present disclosure, the terms “individual”, “patient”, or “subject” include mammals. The mammals include, but are not limited to, domestic animals (e.g., cattle, sheep, cats, dogs, and horses), primates (e.g., humans and non-human primates such as monkeys), rabbits, and rodents (e.g., mice and rats).

Unless otherwise defined or clearly indicated by the context, all technical and scientific terms in the present disclosure have the same meaning as commonly understood by a person of ordinary skill in the art to which the present disclosure pertains.

### EXAMPLES

The sequence modification method used in the present disclosure is primarily based on a NEB kit, namely NEBuilder® HiFi DNA Assembly Master Mix, and operations are performed according to the kit instructions. Small-scale plasmid construction involving minor sequence modifications is performed using the Gibson method, and the kit used is NEBuilder® HiFi DNA Assembly Master Mix.

Linearized templates for in vitro transcription are prepared by enzymatic digestion and the restriction site is BspQI. After enzymatic digestion, the templates are recovered by a Sangon PCR purification kit and analyzed by agarose gel electrophoresis or capillary electrophoresis to confirm complete digestion.

The in vitro transcription kit used for in vitro transcription, including a T7 polymerase Mix, a reaction buffer, and the four nucleotides AGCU, purchased from Shanghai Zhaowei Company. The original plasmid template is purchased from GenScript Company.

The preparation of Lipid Nanoparticles (LNPs) is performed by encapsulation using cationic liposomes. Expression validation is performed in Hela cells or 293T cells, and each well of a 96-well plate is transfected with 100 ng of RNA. The electrophoretic separation of circRNA is performed by E-gel EX 2% Agarose from Thermo Fisher Company.

The RNA used in the present disclosure is prepared by in vitro transcription. The in vitro transcription templates used are purchased from GenScript Company. Template linearization is performed by BspQI. The in vitro transcription kit, including a T7 polymerase Mix, a reaction buffer, and the four nucleotides AGCU, is purchased from Shanghai Zhaowei Company. Sequence modification is mainly based on a NEB Q5@Site-Directed Mutagenesis Kit, while minor sequence modifications are performed by the Gibson method, and the kit used is NEBuilder® HiFi DNA Assembly Master Mix. The gel separation of circRNA is performed by E-gel EX 2% Agarose from Thermo Fisher Company.

### Example 1 Feasibility Experiment for Scar-Free Circularization by Modifying IG Sequence

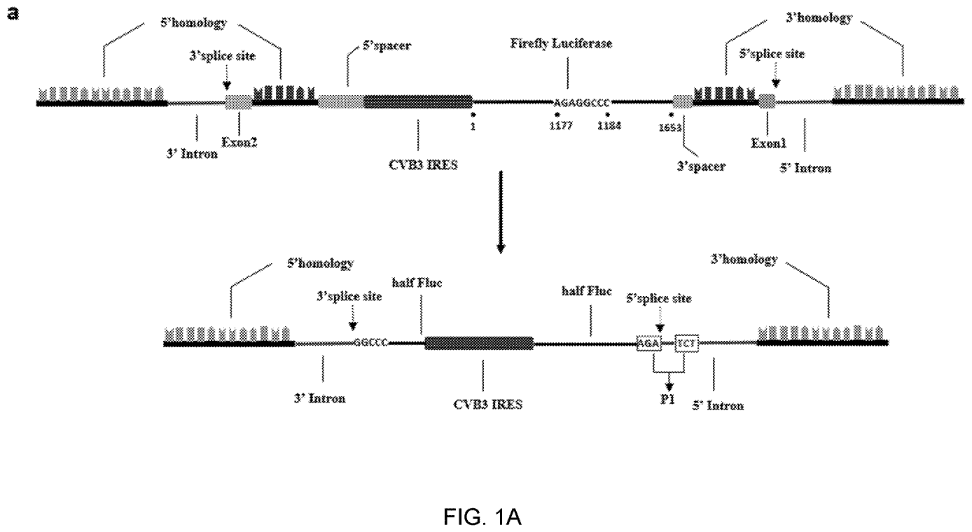

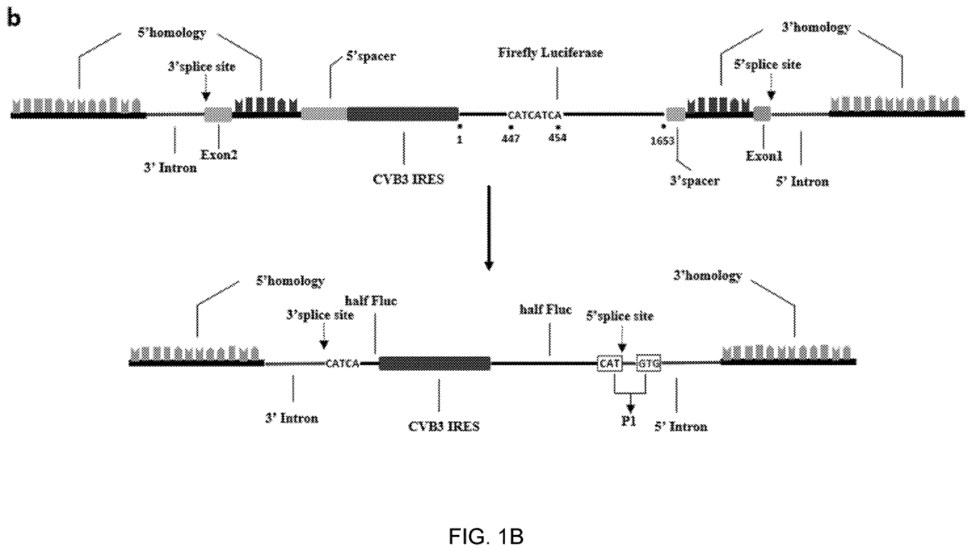

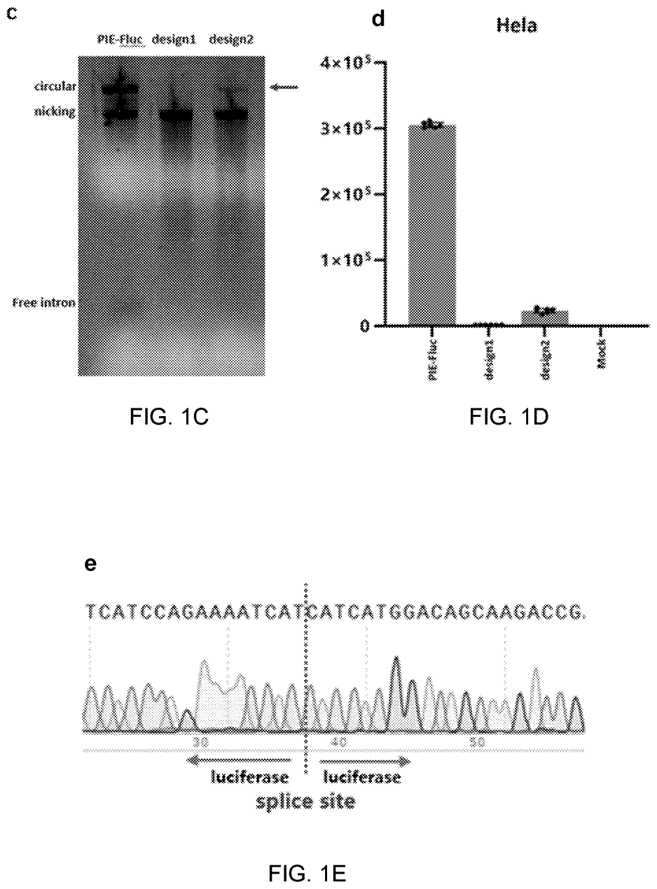

The main elements of the PIE method are shown in the upper part of FIG. 1A. Homology arms are added to both sides, thereby bringing the splice sites closer to each other, which is more conducive to splicing. Spacer sequences are added between an intron and an IRES sequence, thereby separating the two sequences with complex secondary structures, avoiding mutual influence, and thereby improving circularization efficiency. The intron sequences on both sides perform a ribozyme function, and splicing and circularization is performed by the IG sequence in the intron recognizing heterologous exon sequences. The IG sequence of a group I ribozyme from Anabaena tRNA^{leu} used in the present disclosure has a length of 3 nt, and this 3 nt sequence is responsible for recognizing and splicing the splice sites. Therefore, it is first planned to modify the IG sequence, thereby causing it to recognize a segment of sequence located in an ORF, and thus not depend on the heterologous exon sequences for a circularization reaction. Here, Firefly Luciferase (1653 nt) was used as a reporter gene in the experiments, and two designs were made. Design1 first performed secondary structure simulation to screen sites that are spatially close to each other as splice sites (FIG. 1A). This characteristic is considered to be conducive to circularization. Design2, based on design1, added the characteristic of ‘GU’ wobble pairing (FIG. 1). This characteristic is conserved in a plurality of other group I ribozymes, and thus is considered a relatively important characteristic for the occurrence of the splicing reaction.

After in vitro transcription and circularization, it is found that design1 does not produce circRNA, while design2 produces circRNA. However, it is found by 2% EX-gel electrophoresis (FIG. 1C) that, compared to the PIE method, the yield of circRNA produced by design2 is very low. After transfection into Hela cells for expression analysis, it is found (FIG. 1D) that the yield is only about 10% of that of the PIE method. In addition, reverse transcription PCR sequencing was performed on design2, and it is found that accurate splicing is performed (FIG. 1E). This indicates that design2 indeed produces circRNA and performs accurate splicing, but the yield is currently too low. This may be caused by an unclear understanding of the molecular mechanism of ribozyme action. Therefore, an exploration of the molecular mechanism is planned. It is found that in the PIE system, a polyAC spacer can effectively prevent an intron from forming a complex secondary structure with its adjacent sequence. This secondary structure may affect the formation of the natural secondary structure of the ribozyme or destroy the P1 structure, which is unfavorable for splicing. Therefore, in subsequent experiments, in order to investigate the molecular mechanism, the constructed sequence is similar to that used in the original PIE method, but heterologous exon sequences are removed.

### Example 2 Molecular Mechanism of Circularization by Anabaena Group I Ribozyme

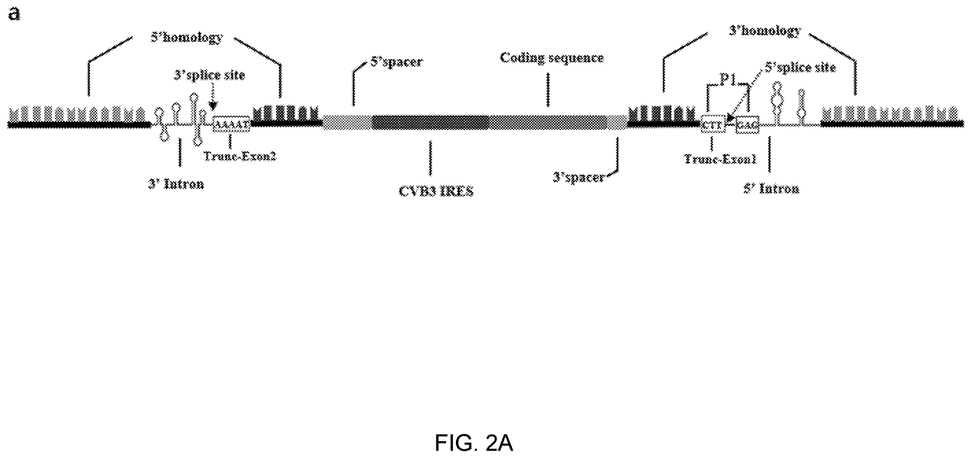

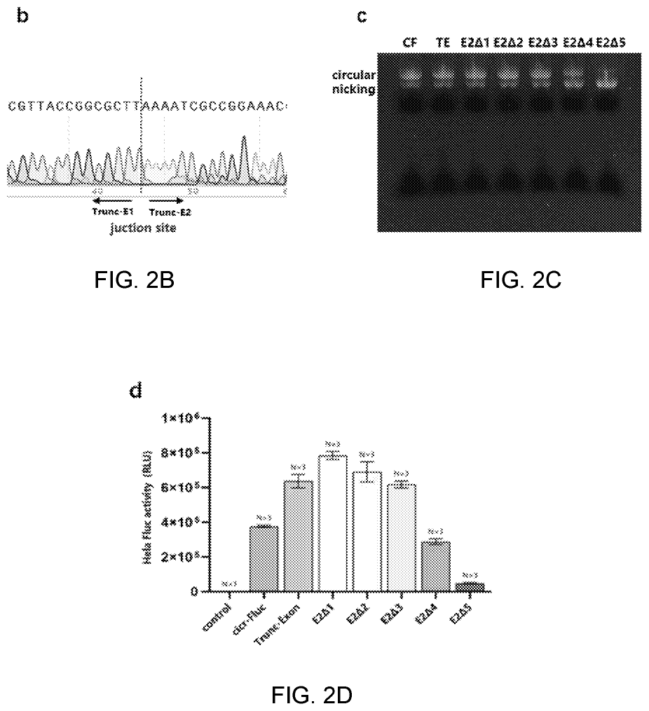

The structures of group I ribozymes are highly conserved, and almost all of them have an IG sequence for recognizing splice sites on both sides. The IG sequence recognizes a 5′ splice site to form P1 and recognizes a 3′ splice site to form P10. However, compared to other group I ribozymes, a group I ribozyme from Anabaena has not been found to have P10, and only a P1 structure formed by a 3 nt of IG sequence and a 5′ splice site is present. This seems to indicate that a group I ribozyme from Anabaena does not need to recognize a 3′ splice site and can perform splicing by simply being adjacent thereto. However, this point is controversial, and some researchers believe that if the group I ribozyme from Anabaena has P10, it may be an “AU” base pair. Due to previous designs only modifying the IG sequence, the circularization rate is too low. Therefore, it is speculated whether a recognition mechanism for a 3′ splice site is present. Therefore, the 5 nt sequence of the original Exon2 as a 3′ splice site in the Anabaena system was first retained for in vitro circularization. This sequence is called “Trunc-Exon” (FIG. 2A). This segment of sequence is successfully circularized, and the circularization efficiency is not reduced compared to the PIE method. After Reverse Transcription Polymerase Chain Reaction (RT-PCR) and sequencing, it is found that this sequence also undergoes correct splicing (FIG. 2B). Then, single nucleotide deletion was performed on this 5 nt sequence. It is found that when four nucleotides are deleted, the circularization efficiency is significantly reduced. When all five nucleotides are deleted, almost no circRNA is produced (FIG. 2C). Cell expression experiments also draw the same conclusion (FIG. 2D). Therefore, a conserved region of 2 nt (AT) is first determined, which is consistent with the previous ‘AU’ base pair conjecture.

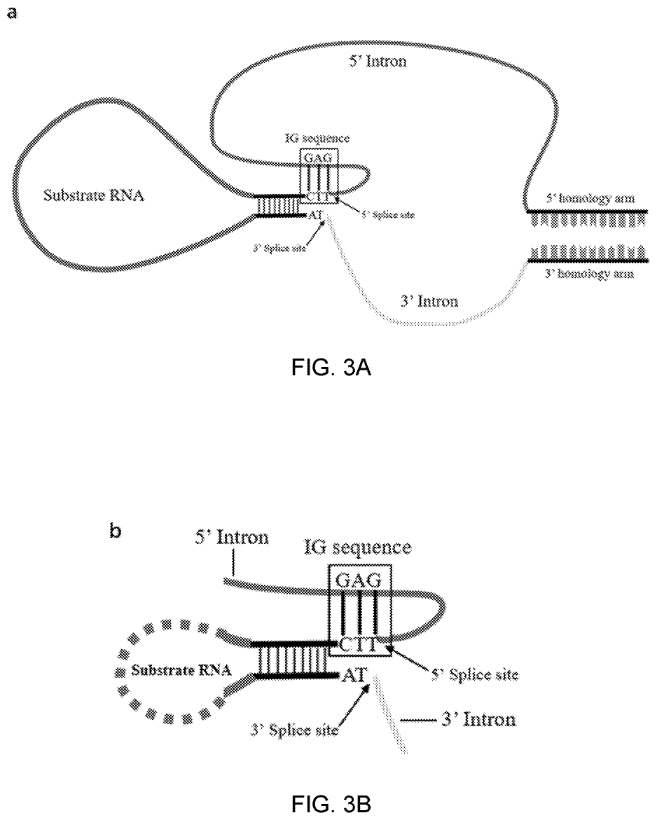

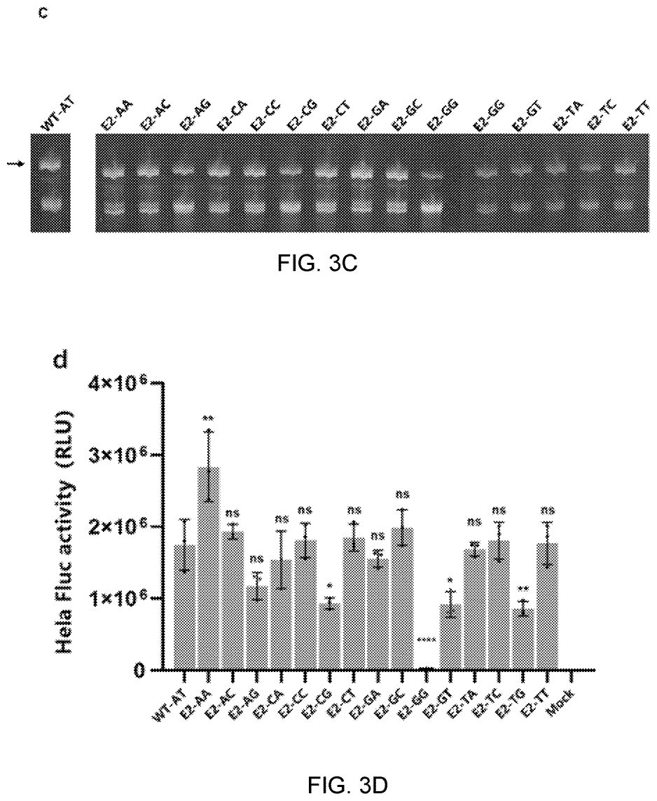

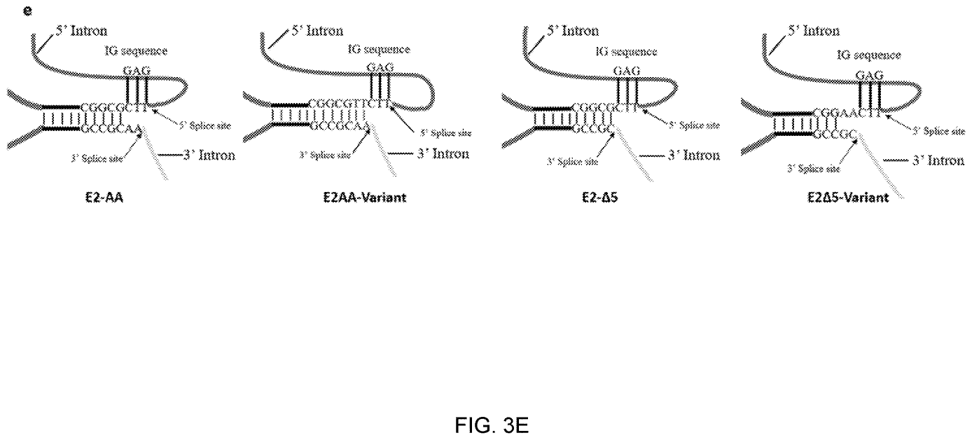

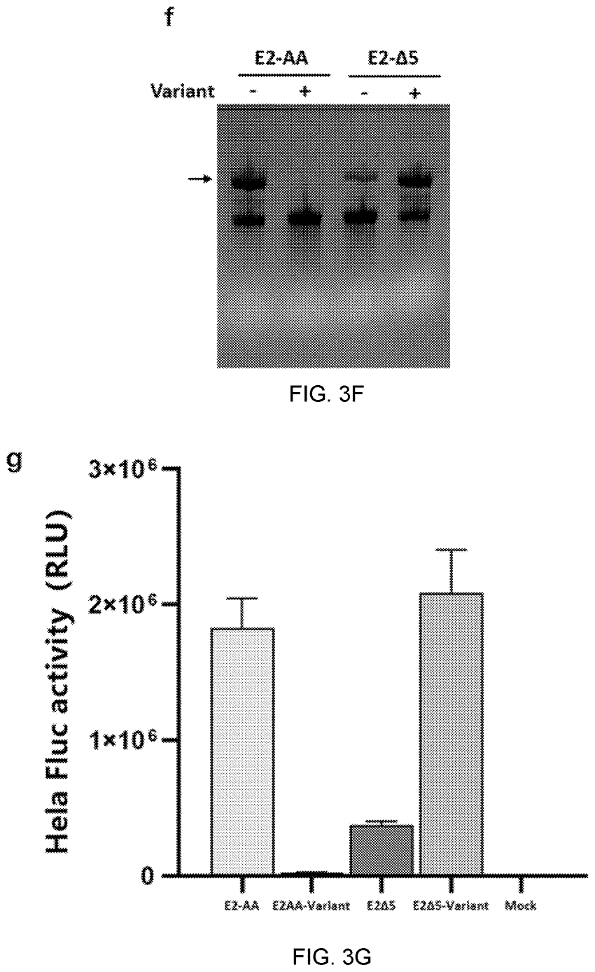

However, in order to further determine whether the nucleotides of the 2 nt conserved region are strictly conserved as “AT”, all other 15 (24-1) possible 2 nt nucleotide combinations were constructed based on the 2 nt structure (FIG. 3A). The results show that almost all 2 nt sequences can be successfully circularized, and the circularization efficiency is not reduced (FIGS. 3C-3D). Only the circularization efficiency of “CG”, “GG”, and “TG” is significantly reduced. This is the same as the previous results of the Exon2A5, because its 2 nt sequence as the 3′ splice site is also “CG”. In the sequences constructed in the present disclosure, Exon2A5, as a 2 nt sequence of the 3′ splice site, is actually located within a 19 nt GC-rich internal homologous sequence. This means that the nucleotides that should serve as the 3′ splice site undergo relatively strong complementary base pairing with the adjacent nucleotides of the 5′ splice site, which is unfavorable for subsequent splicing and may be a reason for the decrease in circularization efficiency.

To verify the above hypothesis, two experiments were planned, a positive one and a negative one. Sequence modification was performed on Exon2-AA with a high circularization rate and the Exon2A5 that cannot be circularized. A “TT” sequence was added to the 5′ splice site to pair with the “AA” of Exon2-AA. Meanwhile, an “AA” sequence was also added to the 5′ splice site of the Exon2Δ5 to disrupt the original “CG” and “GC” pairing (FIG. 3E). The results show that for the Exon2Δ5, when this homology is disrupted, the circularization immediately recovers and exhibits the same circularization efficiency as the original sequence. Conversely, the Exon2-AA that could originally be circularized normally completely lost the circularization capability after the mutation (FIGS. 3F-3G).

This experiment demonstrates that for the Ana system, the 3′ splice site is non-conservative, and there is no other sequence for recognition. However, if the 3′ splice site forms base pairing with the adjacent nucleotide sequence of the 5′ splice site, the circularization efficiency is significantly reduced. This may be due to the fact that when the 3′ splice site forms such base pairing, a relatively tight secondary structure can be formed, thereby making the second splicing process less likely to occur.

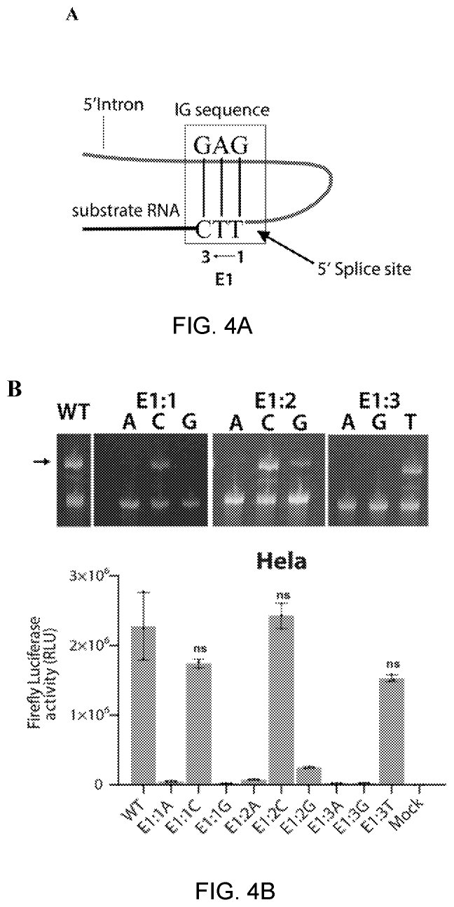

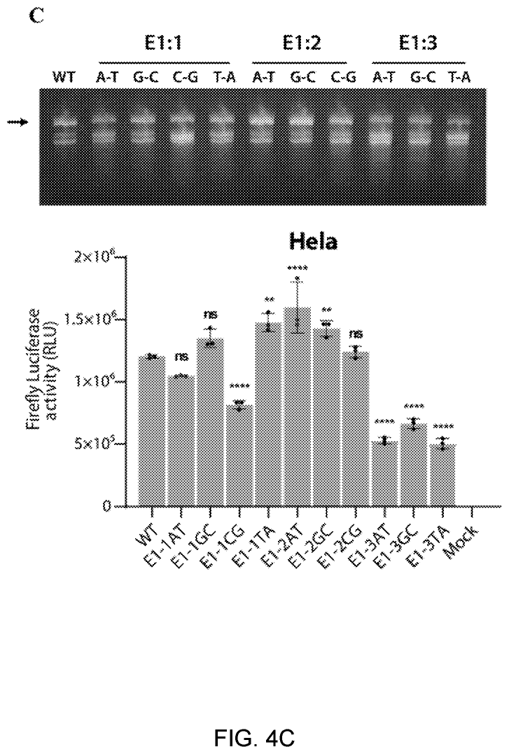

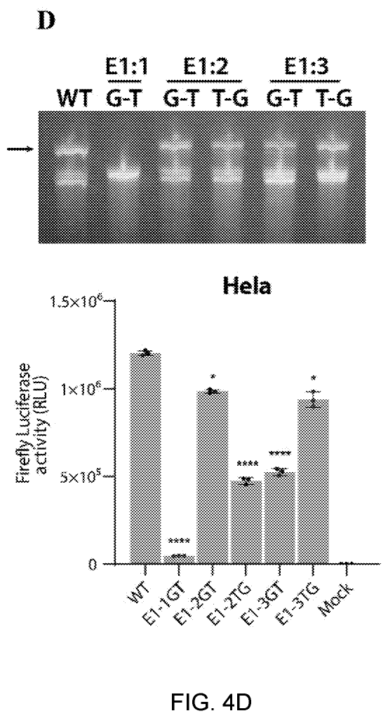

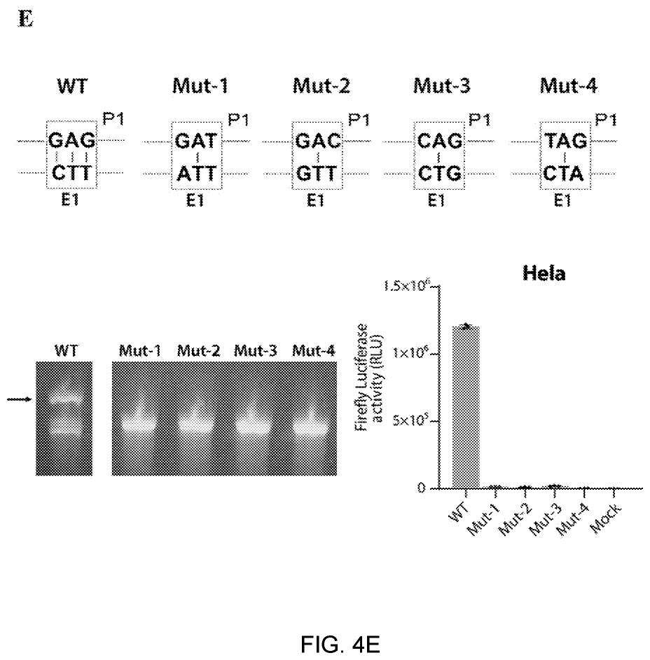

After a detailed study of the 3′ splice site, a study of the 5′ splice site was then conducted. The 5′ splice site is recognized by the IG sequence “GAG” from Anabaena, and the original Exon1 sequence is “CTT”. The Exon1 sequence is ordered from 1 to 3 in a 5′ to 3′ direction (FIG. 4A).

First, in a first experimental step, while keeping the IG sequence unchanged, the Exon1 sequence recognized by it was mutated. For example, the “G” at the first position recognizes the “T” at the first position of the Exon1, the “T” was mutated to the other three nucleotides: “A”, “G”, or “C”. This experiment was performed to investigate the recognition mechanism of the IG sequence for the Exon1 sequence. Electrophoresis results (FIG. 4B) show that the IG sequence recognizes the splice site through complementary base pairing and “GU” wobble pairing, wherein the first and third nucleotides strictly follow this rule, while the second nucleotide does not entirely comply with this rule. For example, the “A-C” combination in the experiment can also undergo splicing and still maintains a high circularization efficiency. This means that the second position of the IG sequence is a non-conservative base position. Cell expression experiments also reflect the same pattern.

The second experimental step is a study of the conservatism of the IG sequence. It can be known from the previous experimental step that normal circularization can occur if complementary base pairing is ensured to satisfy the recognition relationship. Then, when the IG sequence is changed while also ensuring complementary base pairing, does it affect the circularization process? That is, does the IG sequence itself possess a certain degree of conservation? Therefore, a series of subsequent mutations were constructed. For example, the first nucleotide of the IG sequence is recognized through “G-U” wobble pairing, this pair of nucleotides was then modified to “C-G”, “A-T”, “T-A”, and “G-C” to investigate whether it would affect circularization. Electrophoresis results (FIG. 4C) show that when strictly following complementary base pairing, for the base at the second position, the mutation in the IG sequence has no effect on reducing circularization efficiency, and even when mutated to “GC”, the circularization efficiency is even improved. The experimental results of the first-position base mutation show that the modification of the IG sequence has little impact on circularization efficiency, and even if there is some decrease, the impact is not significant. However, mutations in the IG sequence at the third base position all lead to a significant decrease in circularization efficiency. In this experiment, except for the mutation at the third base position where a significant difference can be observed in the electrophoresis diagram, the effects of mutations at other positions are not obvious in the electrophoresis diagram. Therefore, cell expression experiments are crucial for characterizing this circularization efficiency and can quantify the circularization efficiency more clearly.

Furthermore, “GU” wobble pairing is also a possible recognition rule for IG sequence recognition. However, in the original sequence, “GU” wobble pairing only exists at the first position base. Therefore, the effect of “GU” wobble pairing on circularization efficiency was studied at all other positions, and similarly, base pairs at different positions were mutated. It is found that mutating the first position from “T-G” to “G-T” has a dramatic effect on circularization, with almost no circRNA produced. However, such mutations at other positions can all allow circularization, but circularization efficiency decreases to varying degrees (FIG. 4D). Furthermore, it is important to clarify that in the above-mentioned studies, most mutations only reduced circularization efficiency rather than completely preventing the production of circRNA. This is considered to be a fundamental difference. To verify this theory, a group of mutant sequences was therefore constructed, designated as Mut1-4. These sequences do not follow the above-mentioned recognition rules, in addition to maintaining pairing at the non-conservative second base position, the other two positions do not undergo complementary base pairing. After in vitro circularization, it is found that these sequences, which do not follow the recognition rules, produce no circRNA at all (FIG. 4E). This is different from the result of reduced circularization efficiency in the above-mentioned experiments.

Therefore, it is believed that when complementary base pairing or “G-U” wobble pairing is maintained, the decrease in circularization efficiency is caused by a decrease in splicing rate, which is different from the complete absence of circRNA production caused by not satisfying the recognition rule. This decrease in circularization efficiency may be optimized by exploring optimal circularization temperature, metal ion concentration, and circularization reaction time. However, since nicking RNA is generated during the circularization process, it is obvious that a faster splicing rate is more advantageous. Therefore, in combination with the above-mentioned experiments, it is believed that the IG sequence should satisfy the feature of “GNN” and be recognized through complementary base pairing and partial “GU” wobble pairing.

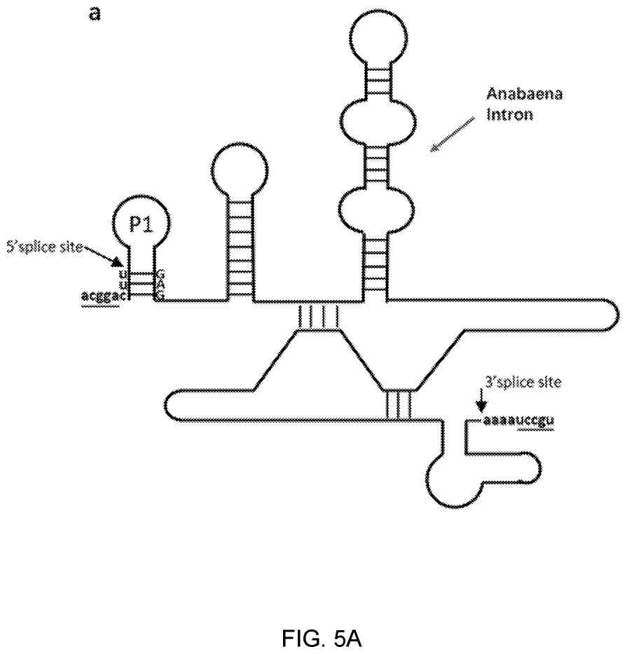

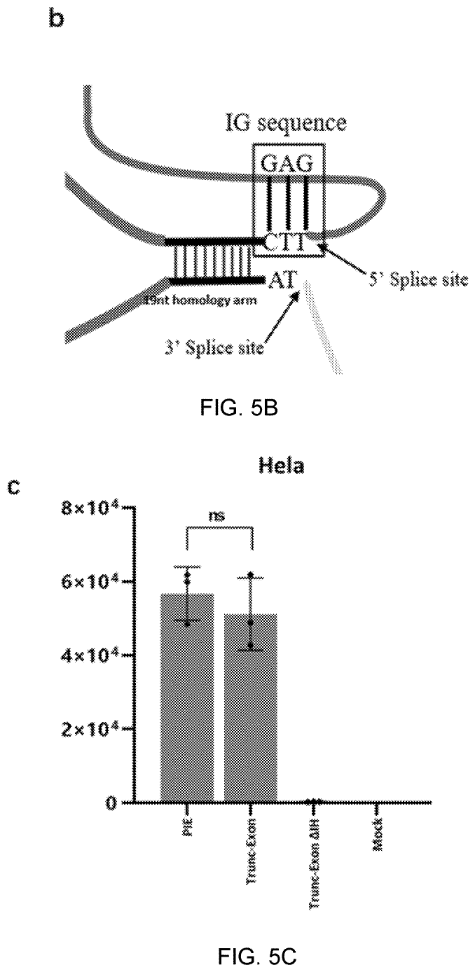

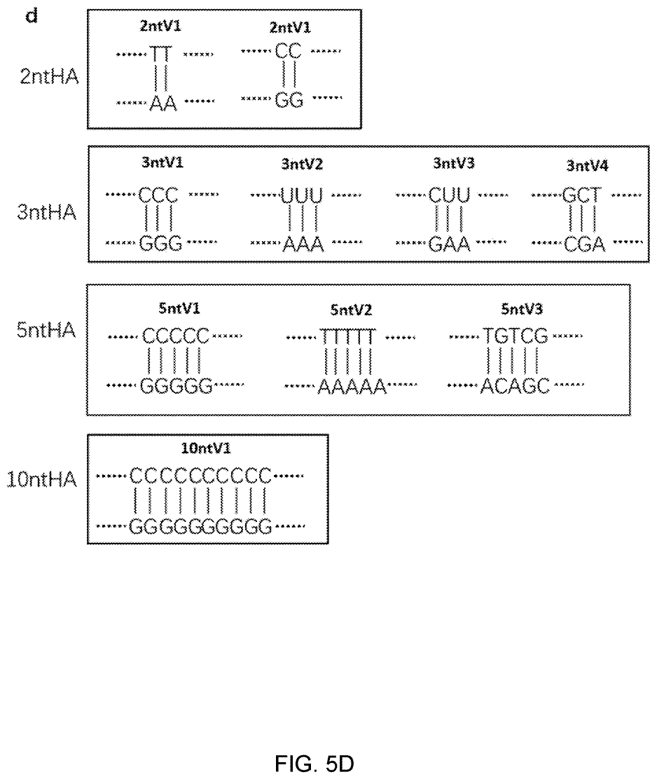

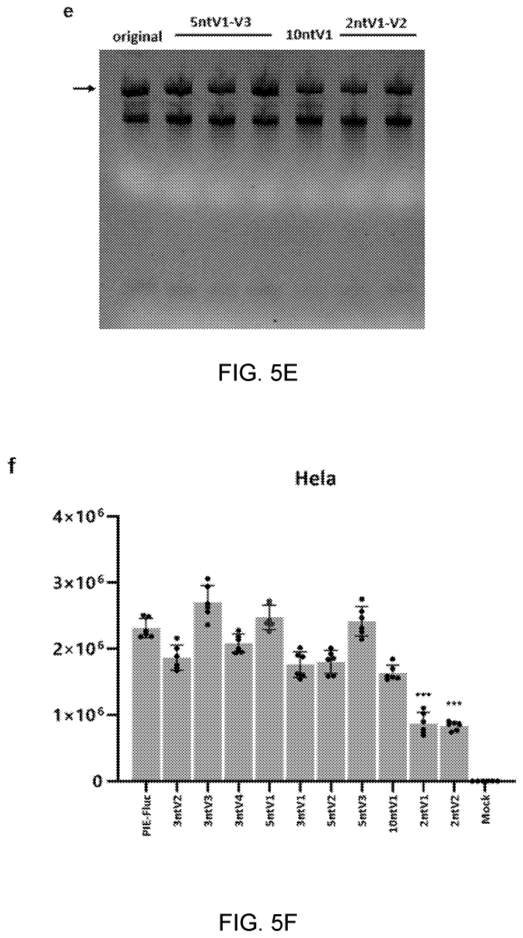

The group I ribozyme system derived from Anabaena was previously studied and found that its adjacent sequences at both splice sites have a 5 nt length of homology. This homology originates from the original Exon of Anabaena (FIG. 5A). Studies (Zaug et al., 1993) indicate that this 5 nt length of homology has a significant effect on circularization efficiency. This may be due to the excessively short IG sequence of the Ana system. In other group I ribozyme systems, the median length of the IG sequence is 5 nt. An excessively short IG sequence leads to instability of the helix formed with the 5′ splice site, requiring an additional homologous sequence to stabilize it. Since this 5 nt homologous sequence is derived from the original heterologous Exon sequence, when scar-free circularization is performed, the heterologous Exon is removed, and this homologous sequence is also removed accordingly. However, in the studied sequences constructed in the present disclosure, a 19 nt length of homology arm exists near the splice site (FIG. 5B). This may also be the reason why previous experiments are not affected. However, after its removal, the results show that no circRNA is produced at all (FIG. 5C).

Experiments show that homology of sequences near the splice site is important for RNA circularization. According to previous studies, the homologous sequence length of the original Exon is 5 nt. Therefore, homologous sequences of different lengths and different GC ratios were constructed based on 5 nt sequence (FIG. 5D) to study their effect on circularization efficiency. The results (FIGS. 5E-5F) show that when there is a 3 nt length of homology arm, the circularization capability is restored, and circularization efficiency is the same as that of the wild type. However, a longer homology arm, such as a 10 nt length, does not improve the circularization capability of circRNA. When the homology arm is shortened to 2 nt, circularization efficiency is significantly reduced. Moreover, there is no significant difference in circularization efficiency between homology arms of the same length but with different GC ratios. This indicates that as long as homology is ensured, no sequence specificity exists.

According to the above-mentioned experiments, several principles affecting the circularization efficiency of in vitro circularization of the Anabaena system were investigated in detail. The following design principles for scar-free circularization are summarized:
(1) The IG sequence needs to satisfy the sequence feature of “GNN”.
(2) When the IG sequence recognizes the 5′ splice site, complementary base pairing or “GU” wobble pairing needs to be ensured.
(3) The 3′ splice site is non-conservative, but when it forms complementary base pairing with sequences near the 5′ splice site, it significantly affects circularization efficiency.
(4) Both splice sites need to have homology arms with at least 3 nt in length for stabilizing the P1 duplex.

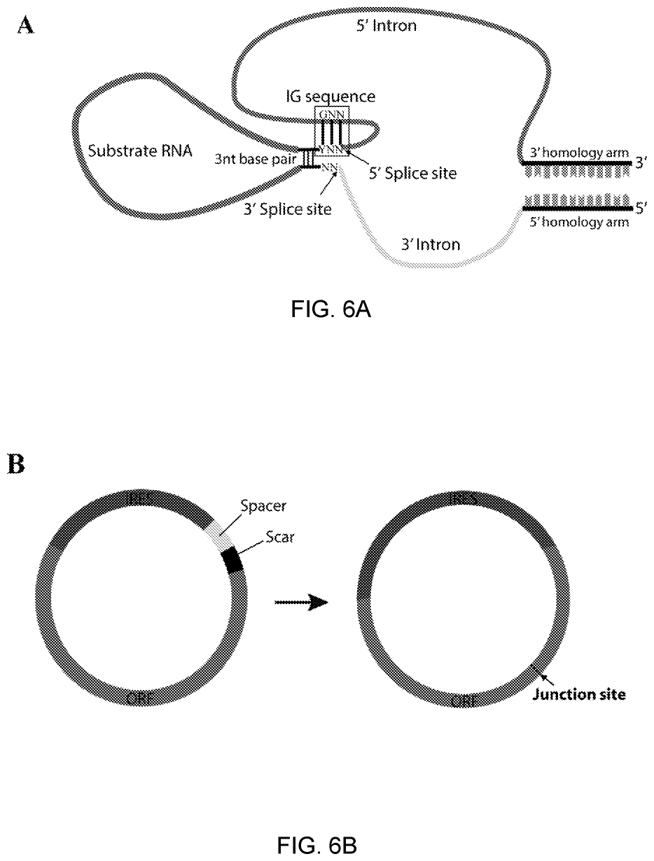

More descriptions regarding the specific structure may be found elsewhere in the present disclosure (e.g., FIG. 6 and related descriptions thereof).

### Example 3 Circularization of IRES+Fluc and IRES+EGFP Using Scar-Free Circularization Strategy Based on Modified Ig Sequence

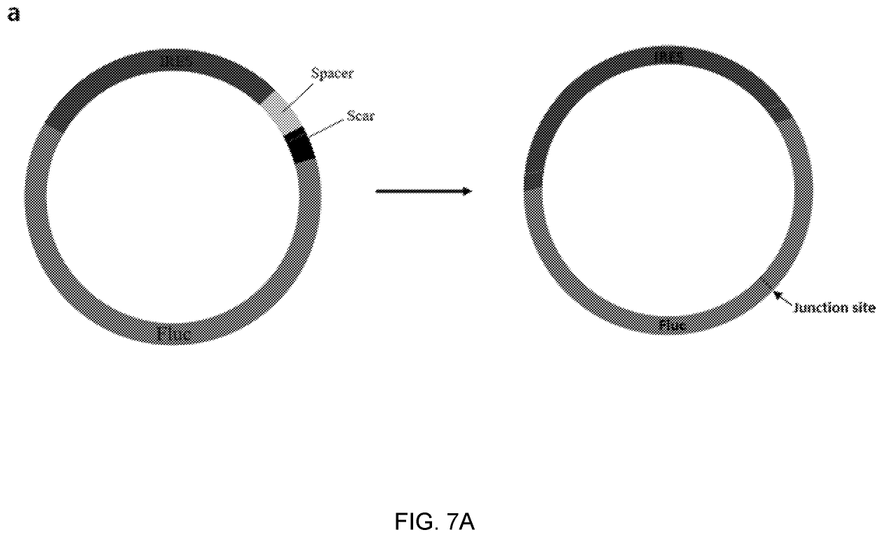

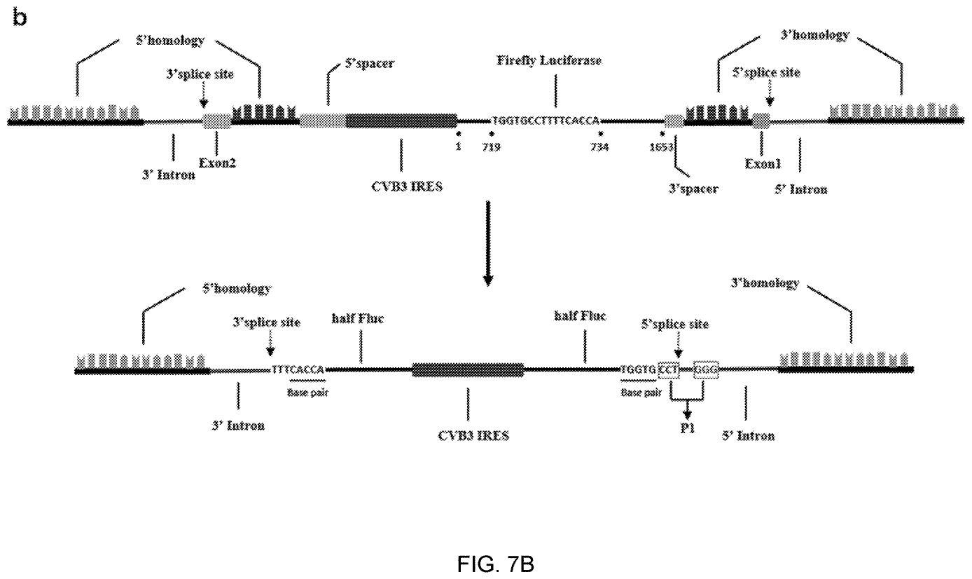

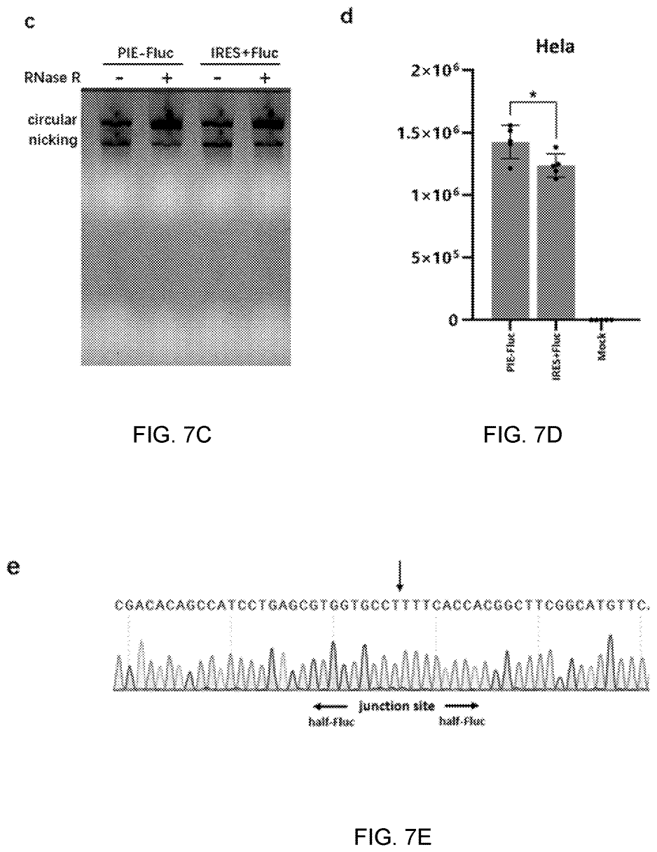

Based on the design principles summarized from previous investigations into the molecular mechanism, first, an appropriate circularization site was found in Firefly Luciferase, then the IG sequence was modified to construct a circRNA containing only “IRES+Fluc” (FIG. 7A). First, a sequence “TGGTGCCTTTTCACCA” from position 719-734 was identified in Fluc, wherein “CCT” can serve as the 5′ splice site, and the IG sequence was modified to “GGG” for recognition; “TT” serves as the 3′ splice site and does not have significant complementary base pairing with nucleotides near the 5′ splice site; and “TGGTG” and “CACCA” on both sides can form a 5 nt length of homologous sequence (FIG. 7B). This sequence is successfully circularized in vitro, and circularization efficiency does not decrease (FIG. 7C). Moreover, it is found through in vitro reverse transcription PCR sequencing that precise splicing is performed (FIG. 7E). However, possibly due to the lack of the polyAC spacer sequence, translation efficiency is slightly decreased (FIG. 7D).

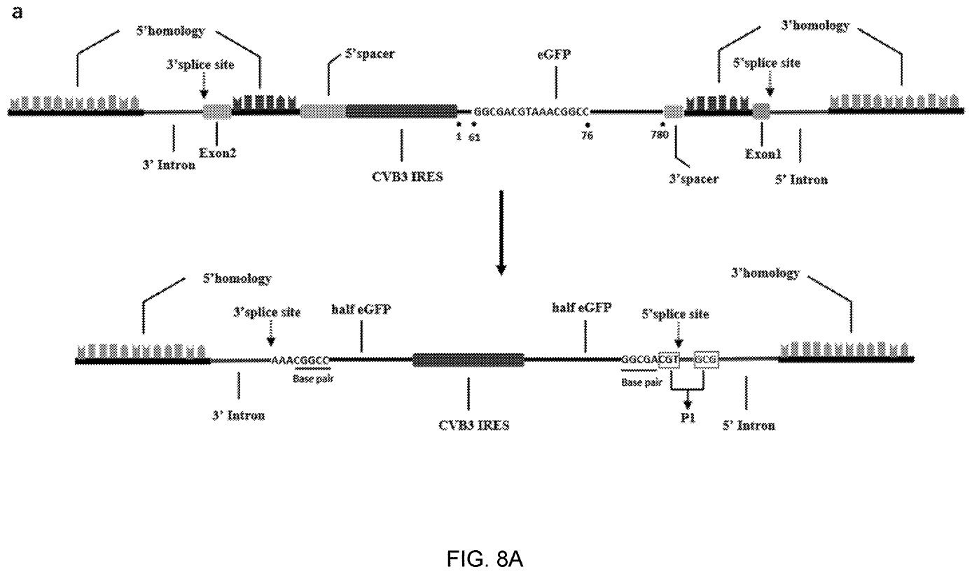

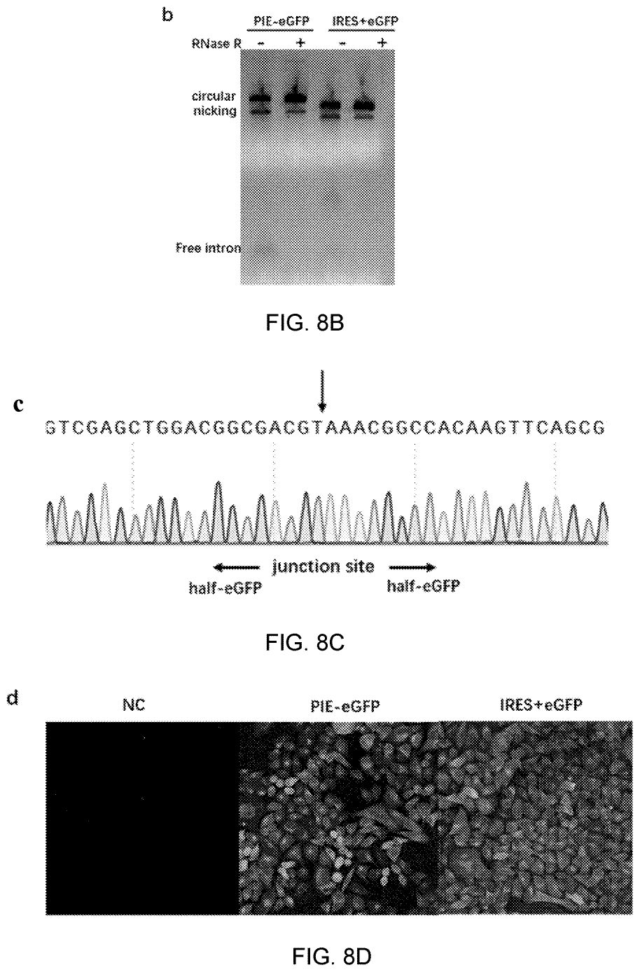

To verify the universality of this modification method, it was planned to perform the same modification on eGFP to construct a circRNA of “IRES+eGFP”. A sequence from positions 61-76 that also conforms to the modification rules was selected in the ORF of eGFP (FIG. 8A), with “CGT” as the 5′ splice site, and the IG sequence was modified to “GCG”. The results show that circular “IRES+eGFP” is also obtained in vitro, and the circularization efficiency is the same as that of PIE method (FIG. 8B). Sequencing results also show that precise splicing is performed (FIG. 8C). Cell expression experiments also demonstrate that the obtained circRNA can express eGFP in cells. However, similarly, compared to the circRNA with polyAC produced by the PIE method, translation is downregulated. The spacer sequence may be important for enhancing the translation capability of circRNA.

According to the above experiments, it is demonstrated that a scar-free circRNA can be successfully prepared in vitro by the above approach. Furthermore, the circularization efficiency is comparable to that of the PIE method, and both approaches are capable of preparing a large-fragment circRNA with high efficiency. However, for different ORFs, it is necessary to select suitable splice sites. Furthermore, the secondary structure of the circularization fragment may affect the functionality of a ribozyme, as has been mentioned in previous literature. This is obviously inconvenient. In addition, our previous studies found that certain spacer sequences can enhance the translational capability of circRNA, which was also confirmed in Howard Chang's research. Therefore, it is planned to screen for spacer sequences that can enhance circRNA translation. Since they have the functionality of enhancing translation, similar to the UTR functionality of linear mRNA, they are hereinafter referred to as UTR sequences. Then, based on the UTR sequence, the IG sequence is modified to construct a universal scar-free circularization method for “IRES+ORF+UTR”.

### Example 4 Screening of UTR Sequences and Construction of “IRES+ORF+UTR” Universal Circularization Method

After researching the mechanism of scar-free circularization via the IG sequence modification, it is planned to screen for UTR sequences that have translation-enhancing effects. This is achieved by modifying the IG sequence to make it recognize the UTR, thereby constructing a universal scar-free system. For different circularization sequences, redesign is not required. The UTR sequences have two functions. First, they can separate the intron sequence from the IRES sequence. These two sequences both have complex secondary structures; if they are adjacent, they could greatly affect the catalytic function of the ribozyme. The second function is to enhance the translational effect of circRNA.

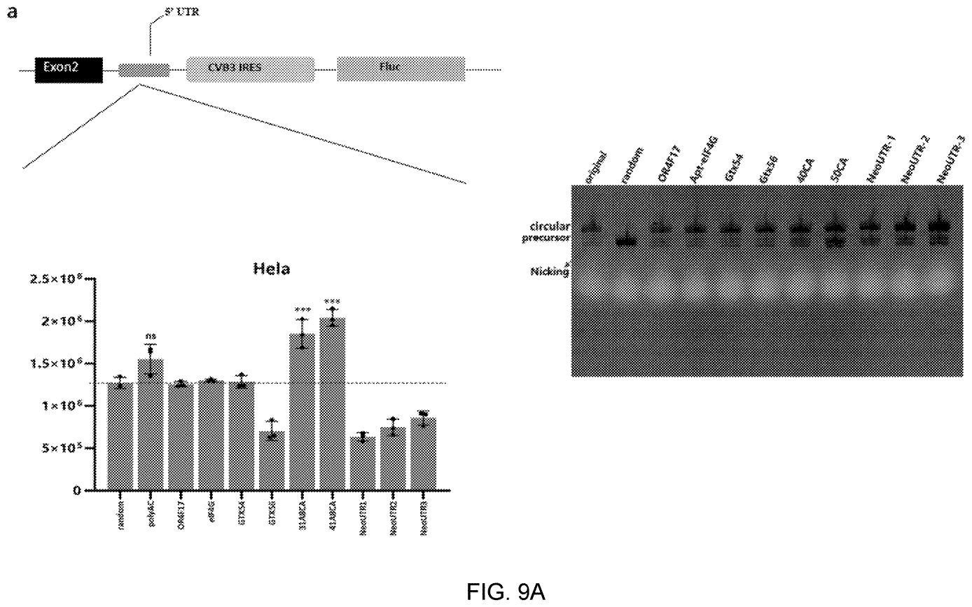

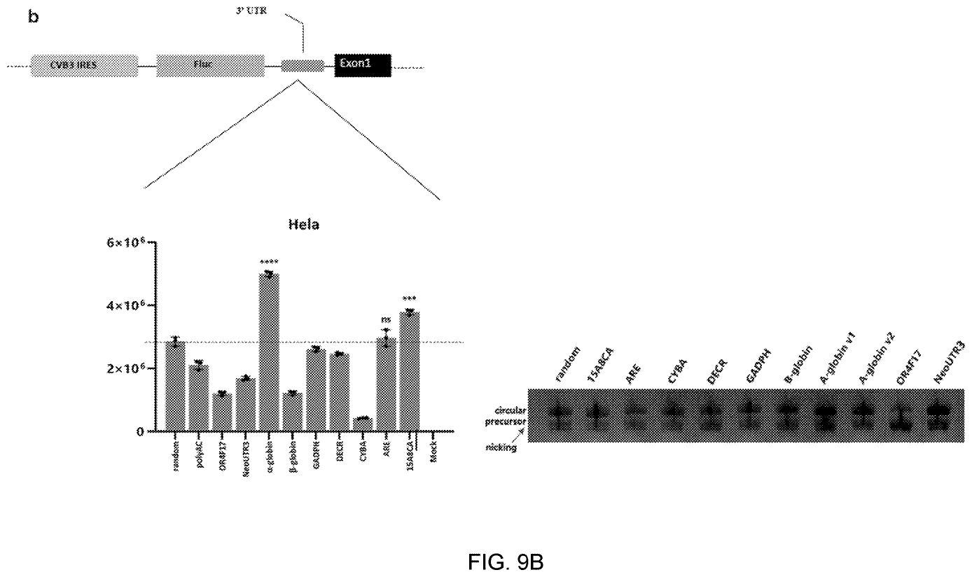

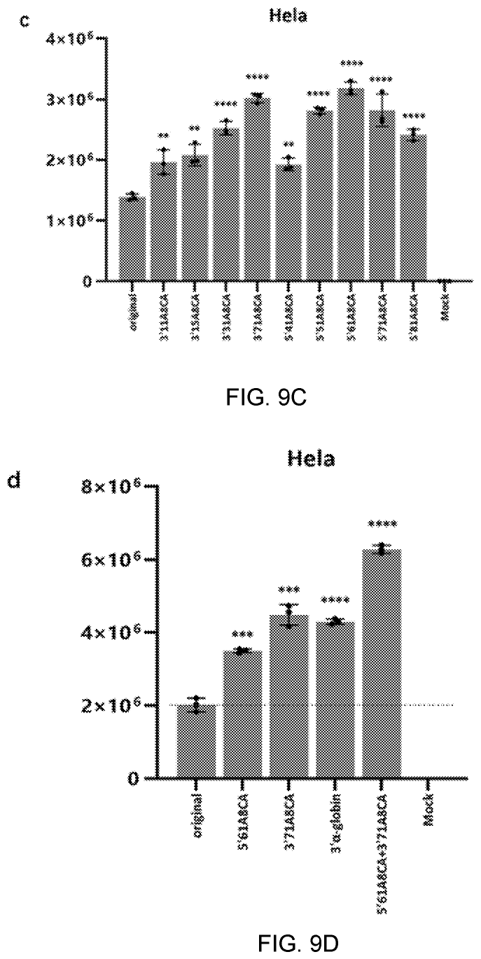

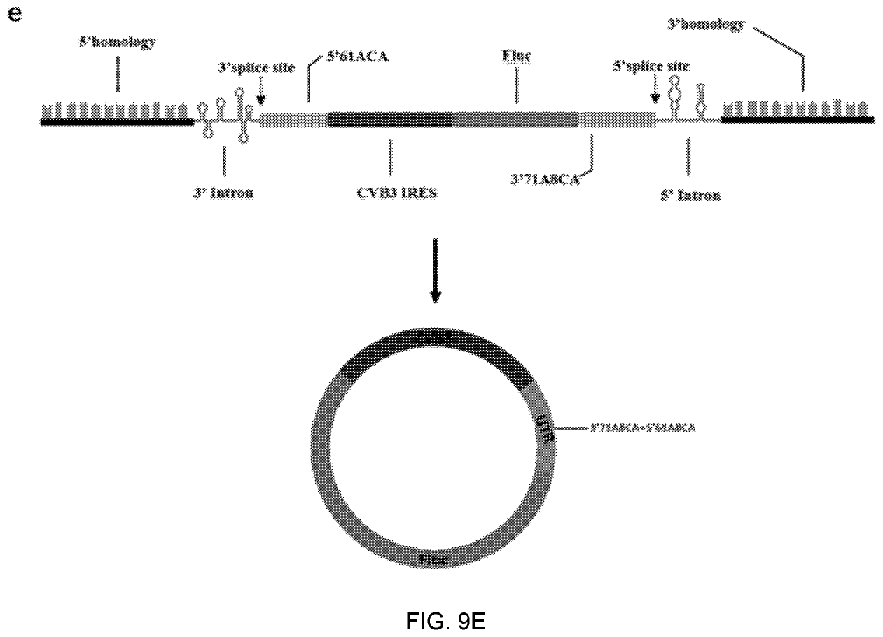

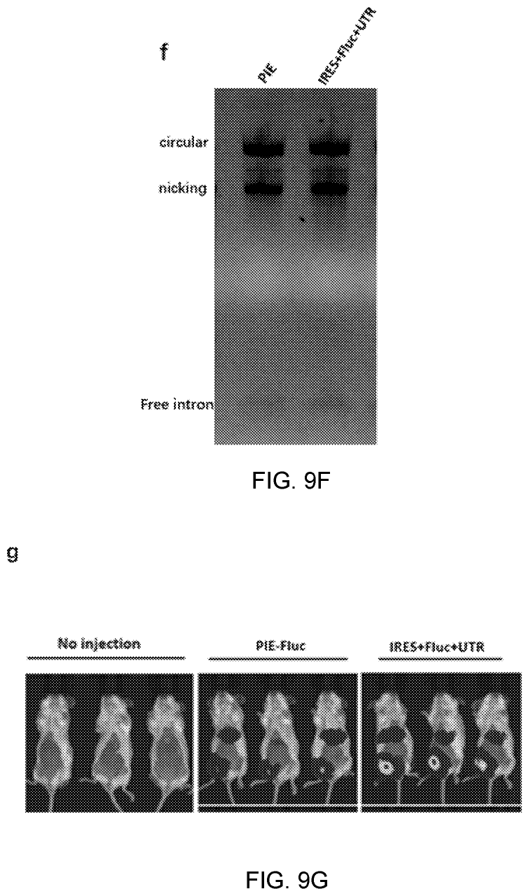

Based on the above principles, the screening of the 5′ UTR was first performed, and three types of sequences were selected. The first type is an IRES enhancer sequence: the IRES sequence binds to 18S rRNA, eIF4G, and eukaryotic initiation factor 4A (eIF4A), thereby initiating translation. If this binding can be enhanced, then the translation efficiency can also be enhanced. Researchers found that there is a 9 nt sequence in the 5′ UTR of the homologous domain protein Gtx, and the sequence is completely paired with nucleotides 1132 to 1124 of 18S rRNA. The article also pointed out that a tandemly repeated 9 nt sequence can greatly enhance the translation efficiency of the IRES sequence. Therefore, the 9 nt sequence was repeated 6 times and designated as ‘Gtx54’ (54 nt). In addition, the author also found in subsequent experiments that a 7 nt sequence within the 9 nt sequence has a more significant enhancing effect on the translation effect. Similarly, after repeated combination, the enhancing effect on the translation effect is more significant. Thus, the 7 nt sequence was repeated 8 times and designated as ‘Gtx56’ (56 nt). Other researchers also found a short ‘IRES-like” sequence that can perform the function of the IRES sequence to initiate translation, and the sequence was designated as “OR4F17” (56 nt). Further, researchers found an aptamer sequence for eIF4G that can recruit eIF4G, thereby enhancing expression, and this sequence was designed as a UTR sequence and designated as “Apt-eIF4G” (51 nt). The second type is a protein-binding sequence. Studies have found that, compared to conventional polyA, inserting cytosine at the tail of a polyA sequence can enhance protein expression, and the sequence can more effectively recruit polyA-binding proteins, thereby achieving an enhanced translation effect. According to the research content of the literature, two sequences, 31A8CA (40 nt) and 41A8CA (50 nt), were selected for research. The third type is a full-length mRNA UTR sequence. Previous studies, through high-throughput screening methods, screened out three UTR sequences that have significant enhancing effects on mRNA from 12,000 sequences. Therefore, experiments were conducted on the enhancing effects of these three sequences on circRNA, which were designated as NeoUTR1 (100 nt), NeoUTR2 (100 nt), and NeoUTR3 (100 nt). In summary, a total of 9 sequences were constructed. In addition, a sequence with a GC content of 50% and a length of 50 nt was also constructed and designated as a random sequence, serving as a control. The original sequence was a polyAC sequence, designated as “polyAC”. After the sequence construction, the influence of these sequences on circularization was analyzed through EX-gel 2% electrophoresis. The gel pattern indicates that these sequences have no effect on the circularization efficiency. Cell expression experiments reveal that 31A8CA and 41A8CA have significant enhancing effects on translation (FIG. 9A).

After screening the 5′ UTR, the 3′ UTR was subsequently screened. For the 3′ UTR, three sequences that have better effects in the 5′ UTR were first selected: OR4F17, 15A8CA, and NeoUTR. Subsequently, a protein-binding sequence, ARE, was selected. The sequence can bind to HuR protein, thereby playing a role in enhancing the translation effect. Finally, five 3′ UTR sequences from highly abundant proteins in mammalians were selected, including GADPH, α-globin, β-globin, CYBA, and DECR1. Similarly, a random fragment with a 50% Guanine-cytosine (GC) content was used as a control, and the original sequence was also “polyAC”. Through EX-gel 2% electrophoresis analysis of the influence of these sequences on circularization, the gel pattern indicates that these sequences have no effect on the circularization efficiency. Cell expression experiments reveal that 15A8CA and α-globin sequences significantly enhance the translation effect (FIG. 9B).

It was pointed out in previous reports that for 41A8CA and 15A8CA, increasing the number of A can enhance their enhancing effect on translation. Therefore, the number of A was gradually increased, and a sequence was designed every 10 A, resulting in a total of 9 sequences. The results show that in the 5′ UTR, as the number of A increases, translation first increases and then decreases, with 61A8CA exhibiting the most significant enhancing effect on translation. In the 3′ UTR, 3′ 71A8CA shows the strongest enhancing effect (FIG. 9C). Finally, the constructed sequence, combining the screened 5′ 61A8CA and 3′ 71A8CA, shows the strongest translation effect, which is increased by nearly 3.7-fold compared to the original sequence (FIG. 9D).

Based on the modification principles of the previous experiments, the UTR sequence and the IG sequence were modified to make them recognize the UTR sequences at both ends. At the third nucleotide position of the 5′ of the 5′ UTR sequence, “CCC” was inserted, two “AA” inherent in 5′ 61A8CA were used as the 3′ splice site, at the 3′ of the 3′ UTR sequence, 6 nucleotides of “GGGCTT” were inserted, wherein “GGG” and the “CCC” at the 5′ constitute the required shortest homology, and “CTT”, serving as a 5′ splice site, is recognized by the “GAG” IG sequence of the ribozyme to complete splicing. According to such a design, the original 108 nt heterologous fragment, including the exon and the internal homologous sequence, can be removed, and only 9 nt of the extra designed sequence needs to be retained to complete circularization, thereby constructing a scar-free circularization system for “IRES+ORF+UTR” (FIG. 9E). Furthermore, 2% EX-gel separation proves that the circularization efficiency is the same as that of the PIE method (FIG. 9F). Introducing the sequence into Balb/C mice, the results show that compared to the original sequence, the translation effect is also significantly enhanced (FIG. 9G).

### Example 5 Enhancement of Translation Efficiency of Circular RNA by IRES Modification

Step 1: Designing a DNA sequence. Using PUC57-Kan as a plasmid vector, XbaLI and PciI were selected as restriction sites on both sides of the target gene. The target gene sequence sequentially comprises a T7 promoter sequence, a homology arm (HA), an intron, a polyAC sequence, a CVB3 IRES sequence, an optimized luciferase sequence, a polyAC sequence, an intron, and a homology arm (HA). Merely by way of example, designing the DNA sequence may include the following steps:

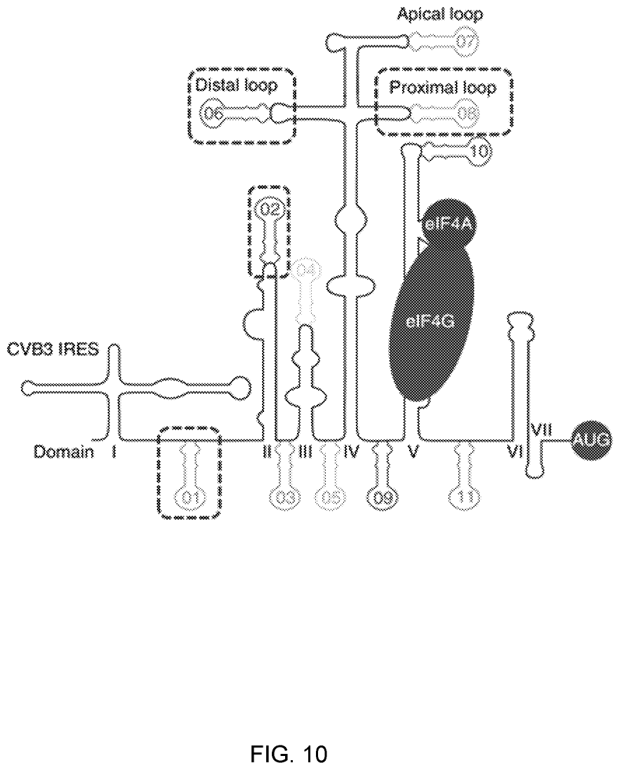

(1). Optimal Position of the IRES Enhancer Element: The present disclosure designed 11 positions for inserting gene sequences. The insertion positions of the IRES enhancer element are shown in FIG. 10. Experimental results found that a total of 4 positions had translation effects. The IRES enhancer element inserted between domain I and domain II was designated as IRES-1. The IRES enhancer element inserted at the stem-loop structure of domain II was designated as IRES-2. The IRES enhancer element inserted at the distal loop of domain IV was designated as IRES-3. The IRES enhancer element inserted at the proximal loop of domain IV was designated as IRES-4. A plasmid without an inserted IRES enhancer element was designated as CVB3 IRES.

(2). IRES Enhancer Elements Screened from a Natural Gene Library: Four sequences were selected from a natural gene library. Except for the DNA sequence of the IRES enhancer element, which was different from IRES-1, all other genes on the plasmid were the same as IRES-1. The four sequences were sequentially designated as Seq IRES A1-4. Specific primer sequences are shown in Table 3, and specific sequences of IRES A1-4 are shown in Table 4.

(3). Investigation of Repeat Design for IRES Enhancer Elements: Repeat design was performed on the screened IRES enhancer elements to investigate the optimal translation efficiency.

(4). The repeated sequences were sequentially designated as IRES-Xn (n: number of repeats). Specific sequences are shown in Table 5.

Step 2: Inserting an IRES Enhancer Element. Primers were designed, and then plasmids with inserted IRES enhancer elements were obtained through PCR. The primer sequences for obtaining the vector by the PCR are shown in Table 1, and the PCR reaction parameters are shown in Table 2.

TABLE 1

| Name | Forward Primer | Reverse Primer |
|---|---|---|
| IRES-1 | TCCCCCAACTGTAAC | GGGGGTATAAAA |
| Vector | TTAGAAGTAACAC | CAGGCGCAC |
| IRES-2 | ACGCGGTTGAAGGAG | GAGCAGTCTATT |
| Vector | AAAG | GATACTCAGTC |
| IRES-3 | CCAGTGTAGATCAGG | GGTAGTGCTGAGCGAA |
| Vector | TCG | |
| IRES-4 | AACCCATGGGACGCT | TCCCCATGGGCAGGCCGC |
| vector | CTAATACAGA | |

TABLE 2

| Reaction step | Temperature | Time | |
|---|---|---|---|
| 1) Pre-denaturation | 98 °C | 30 | s |
| 2) Denaturation | 98 °C | 10 | s |
| 3) Annealing | 60 °C/55 °C/53 °C | 5 | s |
| 4) Extension | 72 °C | 30 | s |
| Steps 2)-4) are cycled 30 times | | | |
| 5) Final extension | 72 °C | 1 | min |
| 6) Hold | 4 °C | 2 | h |

Step 3: PCR reaction purification. The purified vector and IRES enhancer element were obtained using a PCR purification kit according to the manufacturer's instructions. DNA was qualitatively and quantitatively analyzed using an ultramicro ultraviolet spectrophotometer, and agarose gel electrophoresis was performed to verify the integrity and purity of a DNA template.

Step 4: transforming a PCR product into competent cells.

In some embodiments, the specific steps for transforming the PCR product into the competent cells include:

(1) E. coli DH5a competent cells were thawed on ice before use.

(2) 2 μL of the PCR product was added to 50 μL of the cells, and the tube wall was gently flicked to mix.

(3) After gentle mixing, the cells were placed on ice.

(4) The cells were incubated on ice for 30 min, then rapidly subjected to a heat shock in a 42 °C water bath for 45 s, and then rapidly transferred to ice to stand for 2 min.

(5) An SOC medium was added to supplement the volume to 1 mL.

(6) The cells were shaken and cultured at 37 °C for 1 h.

(7) Concentration: The culture was centrifuged at 5000×g for 1 min, 900 μL of supernatant was discarded, and the remaining part was thoroughly mixed by pipetting.

(8) 100 μL of the bacterial solution was spread onto a Luria-Bertani (LB) plate with 100 mg/L Kanamycin and incubated upright at 37 °C until the bacterial solution was absorbed.

(9) The plate was cultured overnight inverted at 37 °C.

(10) The next day, single colonies were picked from the plate and inoculated into a LB medium containing Kanamycin, and thoroughly shaken and cultured in a shaker at 37 °C for 12 to 16 h.

Step 5: In vitro transcription of circRNA: Plasmids were extracted, and the DNA template was linearized by enzyme digestion. An in vitro transcription kit was used and transcription substrates including RNA transcriptase, nucleoside triphosphates, and the DNA template, were added in vitro. After incubation at 37 °C for 2 h, nuclease-free water and GTP were added, followed by incubation at 55 °C for 30 min, thereby transcribing circRNA with the DNA as a template.

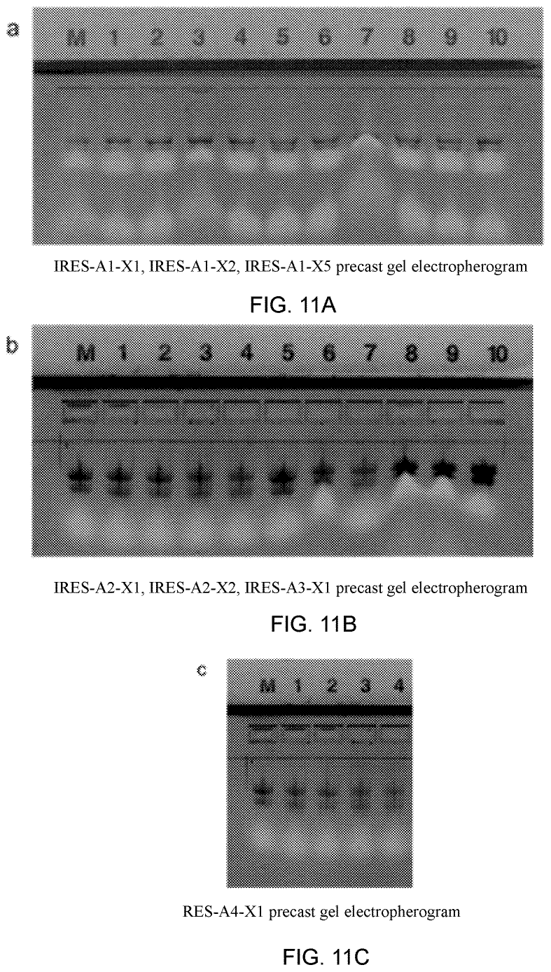

Step 6: pre-cast gel electrophoresis verification: The circularization efficiency of the synthesized circRNA was detected, and the pre-cast gel electrophoresis pattern is shown in FIG. 11.

Step 7: Liposome preparation: Liposomes were prepared according to the following ratio. The molar ratio of a cationic liposome, a helper lipid, cholesterol, and polyethylene glycol was 50:10:38.5:1.5. The cationic liposome is SM-102, the helper lipid is dioleoyl phosphatidylethanolamine (abbreviated as DOPE), and the polyethylene glycol is dimyristoyl glycerol-polyethylene glycol 2000 (abbreviated as DMG-PEG 2000).

Step 8: Synthesis of LNPs: According to a volume ratio of the liposome to mRNA of 3:1, a desired volume of the nanoparticles was prepared, gently shaken and mixed, thereby preparing the LNPs required for cell transfection.

Step 9: Cell transfection. Cells were plated the day before transfection. After the cell state was observed and the cell density grown to approximately 70%-90%, the synthesized circRNA containing IRES enhancer element insertion sites was transfected into HeLa and HEK-293T cells. Specifically, circRNA IRES-A1-X2, IRES-A1-X5, IRES-A2-X2, IRES-A3-X1, and IRES-A4-X1 were transfected into the HeLa and HEK-293T cells. After the transfection, the cells were gently shaken and mixed, and cultured in a cell incubator.

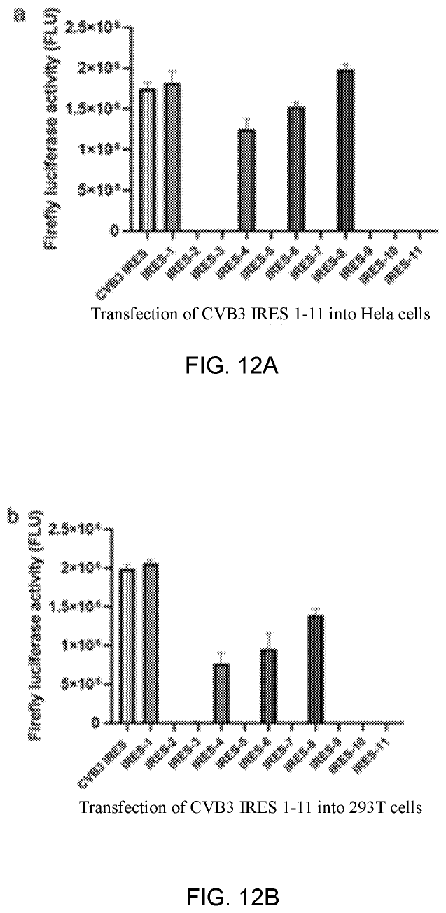

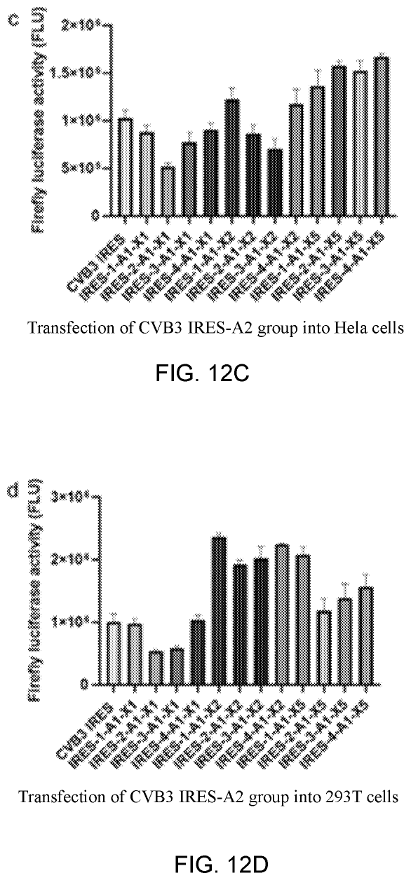

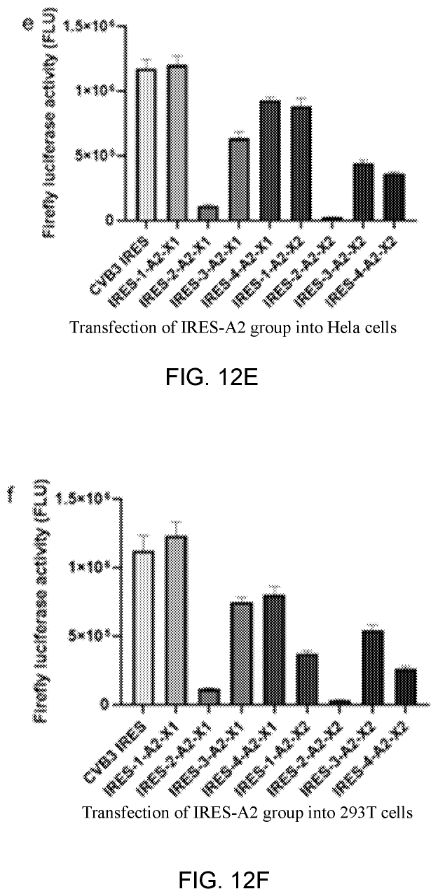

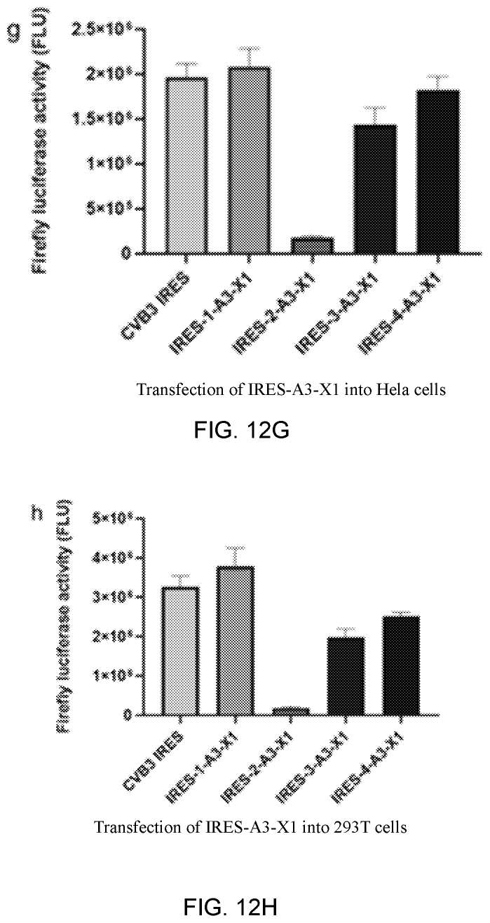

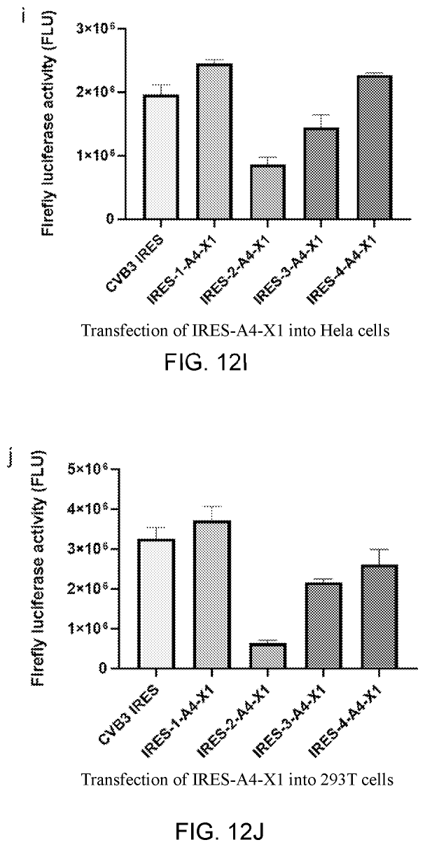

Step 10: Detection of fluorescence intensity after 24 h. After 24 h of the transfection, the plates were removed. After balancing to room temperature, a luciferin substrate was added. After the cells were fully lysed for 10 min, a luminescence signal was detected with a microplate reader. Results of exploring the optimal insertion position of the IRES enhancer element, transfection results of IRES A1-4, and transfection results of IRES-A1-X2, IRES-A1-X5, IRES-A2-X2, IRES-A3-X2, and IRES-A4-X2 are shown in FIG. 12.

Additionally, the present disclosure also modifies the CVB3 IRES sequence. First, the present disclosure investigated an optimal position for inserting an IRES enhancer element into the CVB3 IRES sequence. The present disclosure designed eleven types of positions for inserting gene sequences, and experiments found that four types of insertion positions have a translation effect. The four specific insertion positions are respectively as follows: between the domain I and the domain II of the IRES sequence, designated as IRES-1; at a stem-loop structure of the domain II of the IRES sequence, designated as IRES-2; at a distal loop of the domain IV of the IRES sequence, designated as IRES-3; and at a proximal loop of the domain IV of the IRES sequence, designated as IRES-4. The IRES enhancer element can enhance the binding of the CVB3 IRES sequence with ribosome, thereby promoting translation of circRNA. The IRES enhancer element is present in circRNA, folds into a structure similar to an initial tRNA, recruits more ribosomes and binds translation regulatory factors such as ITAF, and then introduces the ribosomes into an interior of circRNA to bind and initiate protein translation. The ribosome is a highly diversified protein structure found in all cells. Under the action of translation regulatory factors such as ITAF, the ribosomes are introduced into an internal structure of circRNA to bind and initiate. Second, the present disclosure screened four IRES enhancer elements and then explored an effect of IRES enhancer elements having different sequences with the same enhancement effect on the translation effect of circRNA. The four IRES enhancer elements all have a ribosome recruitment effect, thereby affecting the translation of circRNA. The four screened IRES enhancer element sequences were respectively cloned into a DNA template, the composition of which is the same as described above. Screening for an optimal sequence was performed to obtain a gene sequence with high protein expression. Finally, based on the optimal position of the IRES enhancer element and a basis of screening the IRES enhancer element, the present disclosure performed a series of repeat sequence designs on the IRES enhancer element, thereby obtaining a sequence that enables the IRES enhancer element to achieve its function. Among the screened sequences, IRES-1-A1-X2 was increased by approximately 2.3-fold compared with an original sequence.

The IRES enhancer element is a sequence set forth below:

| Serial | |
|---|---|
| No. | |
| No. | IRES Sequence |
| A1 | CCGGCGGGT |
| A2 | GTTTCATTTTATTCCTATAC |
| | (SEQ ID NO: 46) |
| A3 | CAATTGAGAGATCGTTACCA |
| | TATAGCTA (SEQ ID NO: 47) |
| A4 | ATTTTATTCCTA (SEQ ID NO: 48) |

or repeat sequences of the above sequence:

| Serial | IRES Repeat Sequence |
|---|---|
| A1-X2 | CCGGCGGGTCCGGCGGG |
| | T (SEQ ID NO: 49) |
| A1-X5 | CCGGCGGGTCCGGCGGG |
| | TCCGGCGGGTCCGGCGG |
| | GTCCGGCGGGT |
| | (SEQ ID NO: 50) |
| A2-X2 | GTTTCATTTTATTCCTA |
| | TACGTTTCATTTTATTC |
| | CTATAC (SEQ ID NO: 51) |

### Example 6 Development of Circular RNA Purification Process

Related literature has reported the application of arginine in a purification process for biological products. The mechanism of action of arginine mainly includes: (1) it can inhibit the formation of nucleic acid aggregates and promote complex dissociation; and (2) the addition of arginine increases the overall hydrophobicity of the solution, thereby promoting hydrophobic binding between the aggregate-nucleic acid complex and an affinity probe and reducing non-specific adsorption of an affinity chromatography resin.

Related literature has reported the application of PEG in a purification process for biological products. In addition to forming a depletion zone around solutes, studies have found that PEG is also ‘excluded’ from hydrophilic surfaces, such as most chromatography media. The addition of PEG to a mobile phase in size-exclusion chromatography can lead to a delay in retention time, while also enhancing the binding of substances on ion exchange and affinity chromatography resins.

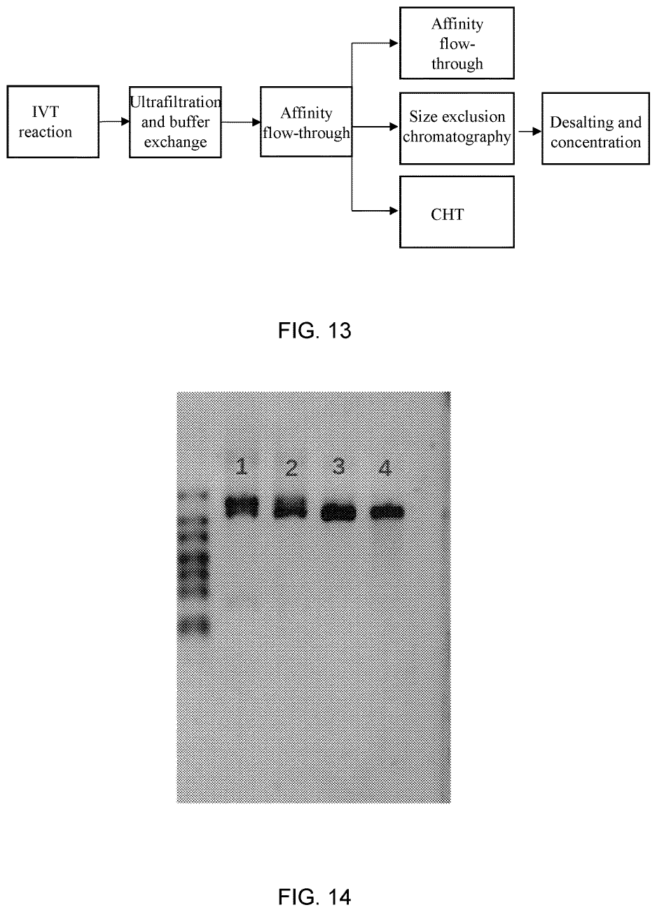

By adding certain concentrations of arginine and PEG to a buffer solution during an ultrafiltration and purification process, the separation degree of collected peaks can be improved, and non-specific adsorption of nucleic acids can be reduced, thereby allowing the purity and overall yield of the circRNA to meet requirements for industrial scale-up. FIG. 13 shows a purification process flow for circRNA.

The selection and precision of IVT reaction equipment determine the yield of an IVT reaction. Common in vitro transcription (IVT) reaction systems include a constant temperature shaking metal bath, a reactor, a temperature-controlled tank, and a waver shaker, or the like.

Commonly used equipment brands include Thermo Scientific (model: Compact) for constant-temperature shaking metal baths, Mettler Toledo (models: EasyMax102 and EasyMax402) and Radeys (model: Mya4) for reactors, LePure and BioLink (model: compatible with 1000 mL and 500 mL disposable square PC bottles, with magnetic stirring, custom type) for temperature-controlled tanks, such as Water-cooled Temperature-controlled Lexiaobao, and Sartorius, LePure, and BioLink (model: Waver10) for Waver shakers.

The reaction conditions controlled by the constant temperature shaking metal bath, the reactor, and the temperature-controlled tank are as follows:

| Control Condition | Control Range | | Example | |
|---|---|---|---|---|
| Temperature | 36 °C-40 °C | | 37 ± 1 | °C |
| Stirring | 10-50 | rpm | 20 rpm or 10-50 rpm gradual | |
| | | | stirring | |
| pH | 8.0-9.2 | | 8.5 | |
| Conductance | 20-200 | us/cm | 52.8 | us/cm |

The reaction conditions of the Waver 10 shaker are controlled as follows:

| Control condition | Control range | | Example | |
|---|---|---|---|---|
| Temperature | 34 °C-40 °C | | 37 ± 2 | °C |
| Stirring | 5-15 | rpm | 10 revolutions per minute (rpm) | |
| | | | or gradient stirring of 5-15 rpm | |
| Rotation Angle | 2-8° | | 5° | |
| pH | 8.0-9.2 | | 8.5 | |
| Conductance | 20-200 | us/cm | 52.8 | us/cm |

The IVT reaction system for circular nucleic acids comprises template DNA, T7 enzyme, dNTP, pyrophosphatase (PPI), PEG, or the like. It is required that the template DNA has a homogeneity of greater than or equal to 95% and a metal residue content of less than 10 parts per million (PPM); the T7 enzyme is required to be from brands such as Promega, Thermo, and Kaika; the PEG is PGE2000-8000 from Thermo or Sigma, and its function is to increase the viscosity of the IVT reaction system and reduce the fluidity of the reaction solution, thereby preventing Mg²⁺ from randomly attacking circular nucleic acid base sequences during the IVT reaction to control the production of Naked RNA.

The IVT reaction control conditions are shown in the table below; the circularization rate is greater than or equal to 75%, and the content of the Naked RNA is less than or equal to 3%:

| Control Condition | Control Range | | Example (1 L) | |
|---|---|---|---|---|
| Fluc linear plasmid | 10-100 | ng/uL | 50 | ng/uL |
| T7 enzyme | 500-800 | ng/uL | 800 | ng/uL |
| Adenosine Triphosphate (ATP) | 75 mM-100 mM | | 100 | mM |
| Cytidine Triphosphate (CTP) | 75 mM-100 mM | | 100 | mM |
| Uridine Triphosphate (UTP) | 75 mM-100 mM | | 100 | mM |
| GTP | 75 mM-100 mM | | 100 | mM |
| Mg²⁺ Buffer | 12.5 mM-50 mM | | 27.5 | mM |
| RNase Inhibitor (RI) | 0.25 to 0.5 | U/uL | 0.4 | U/uL |
| PPI | 0.2 to 0.8 | U/uL | 0.5 | U/uL |

| | | |
|---|---|---|
| PEG2000-8000 | 0.5% to 10% | 5% |

The circularization steps are as follows:

Step 1: ultrafiltration and buffer exchange using a membrane cassette or a hollow fiber column.

The ultrafiltration membrane cassette is made of PES material, and a pore size range thereof is 100 KD, 300 KD, 500 KD, 750 KD, and 1000 KD.

The hollow fiber column is made of PES material, and a pore size range thereof is 100 KD, 300 KD, 500 KD, 750 KD, and 1000 KD.

The ultrafiltration membrane cassettes and the hollow fiber columns are available from manufacturers such as Cytiva, Sartorius, Cobetter, and Asifab.

Through ultrafiltration or hollow fiber column filtration, impurities such as T7 enzyme, DNA residues, dNTPs, and all or a part of the introns, or the like, can be removed.

Ultrafiltration exchange solution 1: NaCl+EDTA, pH=7.4+0.1.

| Control Condition | Control Range | Example |
|---|---|---|
| Sodium chloride | 20 mM to 1M | 100 mM |
| concentration | | |
| EDTA concentration | 5 mM to 20 mM | 10 mM |

Ultrafiltration exchange solution 2: NaCl+arginine+EDTA, pH=7.4 0.1.

| Control Condition | Control Range | Example |
|---|---|---|
| Sodium chloride | 20 mM to 1M | 100 mM |
| concentration | | |
| Arginine concentration | 20 mM to 1M | 100 mM |
| EDTA concentration | 5 mM to 20 mM | 10 mM |

The volume of the ultrafiltration exchange solution is 10 to 50 times (30 times) the initial volume. The results are shown in FIG. 14.

It can be obtained from animal cell experiments that: precursor linear RNA, introns, aggregates, and circular isomers affect cytotoxicity; open rings affect cell activity, and excessive open rings have strong immunogenicity.

Step 2: NHS activated beads based on 4FF or 6FF matrices wer coupled with oligo probes. The oligo probes are designed based on structures of precursor linear RNA and introns generated by a biological reaction (IVT), typically, employing 1 to 3 probes. The resin is named N-Hydroxy succinimide (NHS) affinity resin. The affinity resin has a particle size ranging from 5 μm to 90 μm, and typically is an affinity anion-exchange resin, adopting an anion flow-through mode.

The NHS activated beads based on 4FF or 6FF are available from Cytiva, Chutian Microbeads, Begelone, and Bailinko, or the like.

Process condition 1: NHS affinity+NHS affinity.

The elution solution: NaCl+arginine+EDTA, pH=7.4+0.1.

| Control Condition | Control Range | Example |
|---|---|---|
| Sodium chloride | 20 mM to 1M | 100 mM |
| concentration | | |
| Arginine concentration | 20 mM to 1M | 100 mM |
| EDTA concentration | 5 mM to 20 mM | 10 mM |

In a first NHS affinity process, 5-20% of A260 flow-through peak signal is discarded. The solution collected from a 5%-20% segment primarily comprises aggregates and precursor linear RNA.

The second NHS affinity flow-through peak is entirely collected.

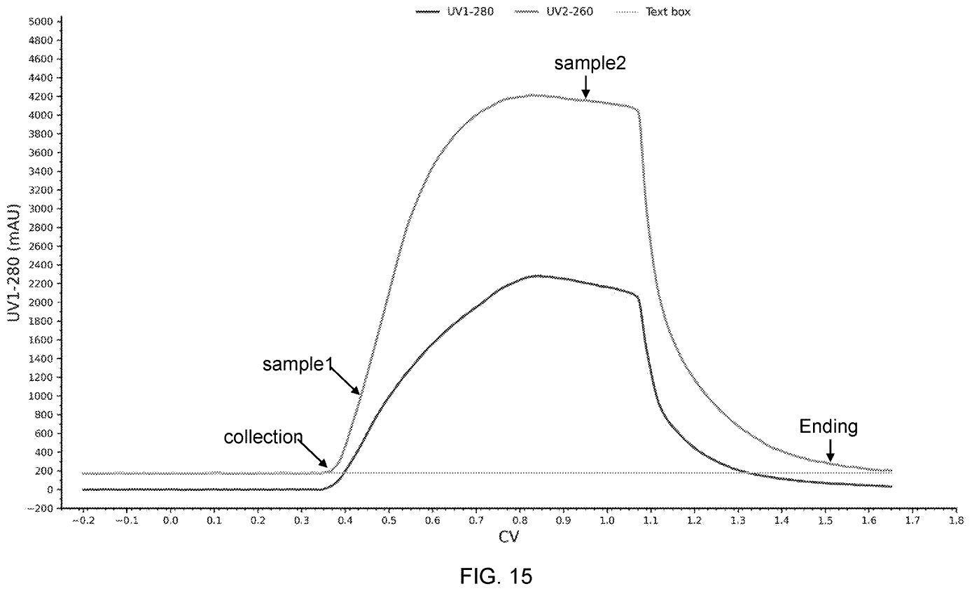

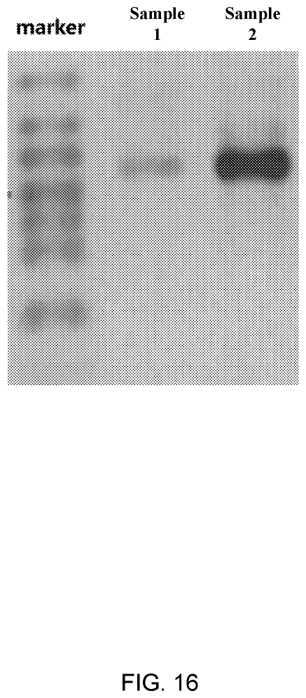

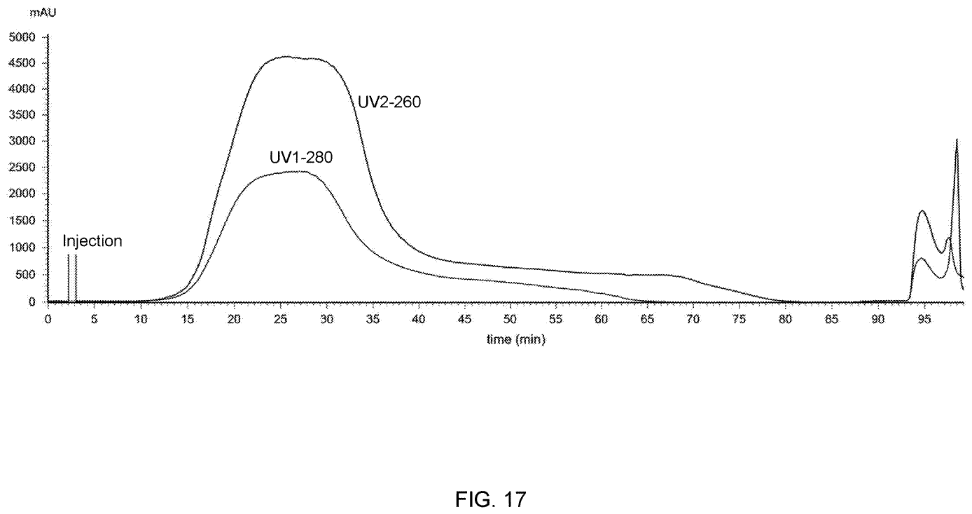

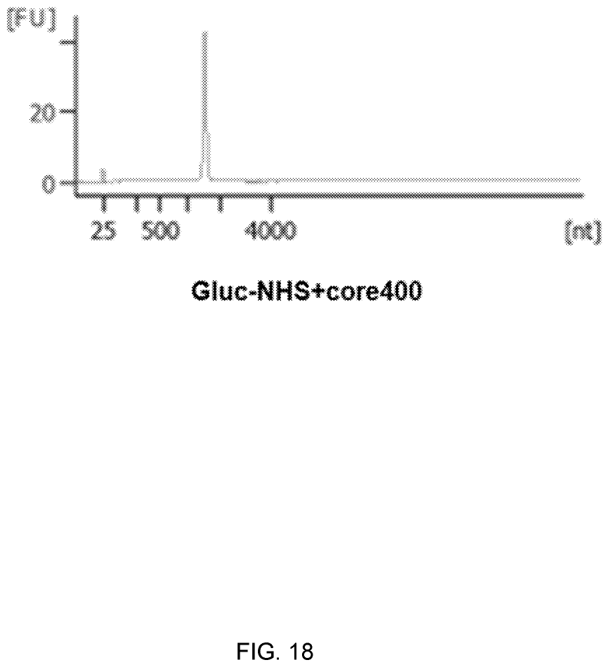

The precursor linear RNA, introns, aggregates, and circular isomers in the samples collected from the two-affinity flow-through processes can be controlled to be less than 1%. A total yield of circular nucleic acids is greater than or equal to 30%, and a theoretical yield of circular nucleic acids is greater than or equal to 70%. Results are shown in FIG. 15, FIG. 16, and FIG. 18.

Process condition 2: NHS affinity+size exclusion chromatography.

The size exclusion chromatography resin includes 4FF, 6FF, core400, core500, core700, core1000, SEC1000 and SEC2000, with a particle size ranging from 5 um to 90 um.

In a first NHS affinity process, 5-20% of an A260 flow-through peak signal is discarded. The solution collected from a 5%-20% segment primarily comprises aggregates and precursor linear RNA.

Size exclusion chromatography: adopting gradient elution.

Equilibration buffer condition: PB+NaCl+EDTA, pH=7.40.1.

| Control Condition | Control Range | Example |
|---|---|---|
| PB concentration | 50 mM to 100 mM | 75 mM |
| NaCl concentration | 0 mM to 200 mM | 100 mM |
| EDTA concentration | 5 mM to 20 mM | 10 mM |

Elution solution condition: PB+NaCl+PEG+EDTA, pH=7.4±0.1.

| Control Condition | Control Range | Example | |
|---|---|---|---|
| PB concentration | 50 mM to 100 mM | 75 | mM |
| NaCl concentration | 200 mM to 2M | 2M | |
| EDTA concentration | 5 mM to 20 mM | 10 | mM |

| | | |
|---|---|---|
| PEG (PEG2000-8000) | 0% to 12% | 12% |

The levels of the precursor linear RNA, introns, aggregates, and circular isomers in the samples collected from a single affinity and size exclusion chromatography can be controlled to be less than 1%, and a small amount of open-circle forms can also be removed. A total yield of circular nucleic acids is greater than or equal to 30%, and a theoretical yield of circular nucleic acids is greater than or equal to 70%.

Process condition 3: NHS affinity+Ceramic Hydroxyapatite (CHT).

In a first NHS affinity process, 5-20% of an A260 flow-through peak signal is discarded. The solution collected from a 5%-20% segment primarily comprises aggregates and precursor linear RNA.

The CHT: adopting gradient elution.

Equilibration buffer condition: PB+NaCl+EDTA, pH=7.40.1.

| | Control | |
|---|---|---|
| Control Condition | Range | Example |
| PB concentration | 50-100 mM | 75 mM |
| Sodium chloride | 0 mM-200 mM | 100 mM |
| concentration | | |
| EDTA concentration | 5 mM-20 mM | 10 mM |

Elution solution condition: Phosphate Buffer (PB)+NaCl+PEG+EDTA, pH=7.4±0.1.

| | Control | | |
|---|---|---|---|
| Control Condition | Range | Case | |
| PB concentration | 50-100 mM | 75 | mM |
| Sodium chloride | 200 mM-2M | 2M | |
| concentration | | | |
| EDTA concentration | 5 mM-20 mM | 10 | mM |

| | | |
|---|---|---|
| PEG (PEG2000-8000) | 0-12% | 12% |

Linear precursor, intron, multimer, and circular isomers in samples collected by a single affinity purification and CHT can be controlled to be less than 1%, and partially open-ring forms can be removed. The total yield of the circular nucleic acid is at least 30%, and the theoretical yield of the circular nucleic acid is at least 70%.

1. A recombinant nucleic acid molecule for preparing circular RNA, wherein in a 5′ to 3′ direction, the recombinant nucleic acid molecule comprises elements operably connected in sequence, the elements comprising an optional 5′ homology arm, a 3′ half-intron fragment, a circularization fragment,
a 5′ half-intron fragment, and an optional 3′ homology arm, wherein a 5′ of the circularization fragment comprises a 3′ circularization recognition segment, and a 3′ of the circularization fragment comprises a 5′ circularization recognition segment;
the 5′ half-intron fragment and the 3′ half-intron fragment are derived from a group I intron, and the group I intron is derived from Anabaena, specifically, Anabaena tRNAleu;
the 5′ half-intron fragment and the 3′ half-intron fragment form an intron sequence in the 5′ to 3′ direction; a nucleotide sequence of the 5′ half-intron fragment comprises a part of the intron sequence proximal to the 5′, a nucleotide sequence of the 3′ half-intron fragment comprises a remaining part of the intron sequence proximal to the 3′, and the 5′ half-intron fragment comprises an internal guide (IG) sequence of the intron sequence;
wherein
(a) the IG sequence of the intron sequence is 3 nt of 5′-GNN-3′, and the 5′ circularization recognition segment is 3 nt of 5′-NNY-3′, wherein Y is C or T/U; N is any nucleotide, and the IG sequence and bases corresponding to the 5′ circularization recognition segment have strict complementary base pairing or GU wobble pairing;
(b) the 5′ of the circularization fragment comprises at least 2 nt of the 3′ circularization recognition segment, and the 3′ circularization recognition segment does not have significant homology with the 5′ circularization recognition segment and adjacent nucleotide sequences; and
(c) an adjacent nucleotide sequence of the 5′ circularization recognition segment and an adjacent nucleotide sequence of the 3′ circularization recognition segment comprise at least 3 nt of an internal homologous sequence, wherein the significant homology refers to complementary base pairing between two or more nucleotides.

2. The recombinant nucleic acid molecule for preparing the circular RNA according to claim 1, wherein the circularization fragment has a 3′ circularization recognition segment-internal homologous sequence segment at 5′ and an internal homologous sequence segment-5′ circularization recognition segment at 3′, with optionally 0, 1, 2, 3, 4, or 5 unpaired bases between the 3′ circularization recognition segment or the 5′ circularization recognition segment and the internal homologous sequence;
preferably, the internal homologous sequence is 3 nt, 4 nt, or 5 nt in length; and/or the 3′ circularization recognition segment has a length of 2 nt.

3. The recombinant nucleic acid molecule for preparing the circular RNA according to claim 1, wherein the circularization fragment comprises the following elements operably linked in sequence:
a first part of a target polypeptide coding region or a non-coding region comprising the 3′ circularization recognition segment, a translation initiation element, and a second part of the target polypeptide coding region or the non-coding region comprising the 5′ circularization recognition segment;
wherein the target polypeptide coding region or the non-coding region is cleaved as follows: the second part containing the 5′ circularization recognition segment I the first part containing the 3′ circularization recognition segment.

4. The recombinant nucleic acid molecule for preparing the circular RNA according to claim 1, wherein the circularization fragment comprises the following elements operably linked in sequence:
a first part of a translation initiation element comprising the 3′ circularization recognition segment, a first part of a target polypeptide coding region or a non-coding region, and a second part of the translation initiation element comprising the 5′ circularization recognition segment;
wherein the translation initiation element is cleaved as follows: the second part containing the 5′ circularization recognition segment | the first part containing the 3′ circularization recognition segment.

5. The recombinant nucleic acid molecule for preparing the circular RNA according to claim 1, wherein the circularization fragment comprises the following elements operably linked in sequence:
a 5′ spacer sequence, an optional translation initiation element, a target polypeptide coding region or a non-coding region, and a 3′ spacer sequence;
preferably, a total number of exogenously introduced bases in the 5′ spacer sequence and the 3′ spacer sequence being less than 10;
preferably, inserting 3 nucleotides at a third or fourth nucleotide position of the 5′ spacer sequence as the internal homologous sequence, using two or three nucleotides carried by the 5′ spacer sequence as the 3′ circularization recognition segment, and inserting 6 nucleotides at 3′ of the 3′ spacer sequence, wherein the 1st-3rd nucleotides are complementary to the nucleotides inserted in the 5′ spacer sequence, and the 4th-6th sequences are NNY, and NNY has strict complementary base pairing or GU wobble pairing with nucleotides corresponding to the internal guide sequence; or
more preferably, inserting 5 nucleotides at 5′ of the 5′ spacer sequence, wherein the 1st-2nd nucleotides serve as the 3′ circularization recognition segment, the 3rd-5th nucleotides serve as the internal homologous sequence, using two or three nucleotides carried by the 5′ spacer sequence as the 3′ circularization recognition segment; inserting 6 nucleotides at 3′ of the 3′ spacer sequence, wherein the 1st-3rd nucleotides are complementary to the nucleotides inserted in the 5′ spacer sequence, and the 4th-6th sequences are NNY.

6. The recombinant nucleic acid molecule for preparing the circular RNA according to claim 5,
wherein the 5′ spacer sequence is selected from:

| | |
|---|---|
| 5′random | GGAGTGCTAGAGGATGACCTTAAATCAGGGACGATATTATAATTGAC |
| | TAA (SEQ ID NO: 8) |
| 5′Apt- | ACTCACTATTTGTTTTCGCGCCCAGTTGCAAAAAGTGTCGTAATTGA |
| elF4G | CTAA (SEQ ID NO: 9) |
| 5′Gtx56 | CCGGCGGCCGGCGGCCGGCGGCCGGCGGCCGGCGGCCGGCGG |
| | CCGGCGGCCGGCGG (SEQ ID NO: 10) |
| 5′Gtx54 | CCGGCGGGTCCGGCGGGTCCGGCGGGTCCGGCGGGTCCGGCG |
| | GGTCCGGCGGGT (SEQ ID NO: 11) |
| 5′OR4F17 | TACTCCCTACTCCCTACTCCCTACTCCCTACTCCCTACTCCCTACTC |
| | CCTACTCCC (SEQ ID NO: 12) |
| 5′NeOUT | CATTCTGTGGTCTGATCATCCTGTGGTTTCGTCGCCGCCATCCTCG |
| R1 | TCGCGACACGCTGTTTTCGGTTCTCGGCCCGACGAGCCATCGCCA |
| | TCCTACAGC (SEQ ID NO: 13) |
| 5′NeOUT | CACTCGCGCTGCCATCACTCTTCCGCCGTCTTCGCCGCCATCCTC |
| R2 | GGCGCGACTCGCTTCTTTCGGTTCTACCAGGTAGAGTCCGCCGCC |
| | ATCCTCCACC (SEQ ID NO: 14) |
| 5′NeOUT | CTTGTCTCGCTCCGGGGAACGCTCGGAAACTCCCGGCCGCCGCC |
| R3 | ACCCGCGTCTGTTCTGTTACACAAGGGAAGAAAAGCCGCTGCCGC |
| | ACTCCGAGTGT (SEQ ID NO: 15) |
| 5′31A8CA | AAAAAAAAAAAAAAAAAAAAAAAAAAAAAAACCCCCCCCA (SEQ ID |
| | NO: 16) |
| 5′41A8CA | AAAAAAAAAAAAAAAAAAAAAAAAAAAAAAAAAAAAAAAAACCCCCC |
| | CCA (SEQ ID NO: 17) |
| 5′51A8CA | AAAAAAAAAAAAAAAAAAAAAAAAAAAAAAAAAAAAAAAAAAAAAAA |
| | AAAACCCCCCCCA (SEQ ID NO: 18) |
| 5′61A8CA | AAAAAAAAAAAAAAAAAAAAAAAAAAAAAAAAAAAAAAAAAAAAAAA |
| | AAAAAAAAAAAAAACCCCCCCCA (SEQ ID NO: 19) |
| 5′71A8CA | AAAAAAAAAAAAAAAAAAAAAAAAAAAAAAAAAAAAAAAAAAAAAAA |
| | AAAAAAAAAAAAAAAAAAAAAAAACCCCCCCCA (SEQ ID NO: 20) |
| 5′81A8CA | AAAAAAAAAAAAAAAAAAAAAAAAAAAAAAAAAAAAAAAAAAAAAAA |
| | AAAAAAAAAAAAAAAAAAAAAAAAAAAAAAAAAACCCCCCCCA (SEQ |
| | ID NO: 21) |

and the 3′ spacer sequence is selected from:

| | |
|---|---|
| 3ARE | TAATATTTATATATTTATATTTTTAAAATATTTATTTATTTATTTATTTA |
| | (SEQ ID NO: 23) |
| 3′CYBA | CCTCGCCCCGGACCTGCCCTCCCGCCAGGTGCACCCACCTGCAATA |
| | AATGCAGCGAAGCCGGGA (SEQ ID NO: 24) |
| 3′DECR1 | GACCACTTTGGCCTTCATCTTGGTTACAGAAAAGGGAATAGAAATGAA |
| | ACAAATTATCTCTCATCTTTTGACTATTTCAAGTCTAATAAATTCTTA |
| | ATTAAC (SEQ ID NO: 25) |
| 3′ | GACCCCTGGACCACCAGCCCCAGCAAGAGCACAAGAGGAAGAGAG |
| GADPH | AGACCCTCACTGCTGGGGAGTCCCTGCCACACTCAGTCCCCCACCA |
| | CACTGAATCTCCCCTCCTCACAGTTGCCATGTAGACCCCTTGAAGAG |
| | GGGAGGGGCCTAGGGAGCCGCACCTTGTCATGTACCATCAATAAAGT |
| | ACCCTGTGCTCAACCAGGAGCTCCC (SEQ ID NO: 26) |
| 3′ | GCTGGAGCCTCGGTGGCCATGCTTCTTGCCCCTTGGGCCTCCCCCC |
| human | AGCCCCTCCTCCCCTTCCTGCACCCGTACCCCCGTGGTCTTTGAATA |
| α- | AAGTCTGA (SEQ ID NO: 27) |
| globin | |
| 3′human | GCTCGCTTTCTTGCTGTCCAATTTCTATTAAAGGTTCCTTTGTTCCCTA |
| β- | AGTCCAACTACTAAACTGGGGGATATTATGAAGGGCCTTGAGCATCTG |
| globin | GATTCTGCCT (SEQ ID NO: 28) |
| 3′OR4F17 | TACTCCCTACTCCCTACTCCCTACTCCCTACTCCCTACTCCCTACTCC |
| | CTACTCCC (SEQ ID NO: 29) |
| 3′NeOUT | CACTCGCGCTGCCATCACTCTTCCGCCGTCTTCGCCGCCATCCTCGG |
| R2 | CGCGACTCGCTTCTTTCGGTTCTACCAGGTAGAGTCCGCCGCCATCC |
| | TCCACC (SEQ ID NO: 30) |
| 3′11A80A | AAAAAAAAAAACCCCCCCCA (SEQ ID NO: 31) |
| 3′15A8CA | AAAAAAAAAAAAAAACCCCCCCCA (SEQ ID NO: 32) |
| 3′21A8CA | AAAAAAAAAAAAAAAAAAAAACCCCCCCCA (SEQ ID NO: 33) |
| 3′31A8CA | AAAAAAAAAAAAAAAAAAAAAAAAAAAAAAACCCCCCCCA (SEQ ID |
| | NO: 34) |
| 3′41A8CA | AAAAAAAAAAAAAAAAAAAAAAAAAAAAAAAAAAAAAAAAACCCCCCC |
| | CA(SEQ ID NO: 35) |
| 3′71A8CA | AAAAAAAAAAAAAAAAAAAAAAAAAAAAAAAAAAAAAAAAAAAAAAAAA |
| | AAAAAAAAAAAAAAAAAAAAAACCCCCCCCA (SEQ ID NO: 36) |

preferably, the 5′ spacer sequence and the 3′ spacer sequence are used individually or in combination; preferably, the 5′ spacer sequence and the 3′ spacer sequence are both a combination structure of polyA+8CA, preferably, the polyA segment contains 10 to 100 A, more preferably, the polyA segment contains 60 to 80 A, most preferably, the 5′ of the polyA segment has 61 A and the 3′ of the polyA segment has 71 A;
most preferably, the 5′ spacer sequence and the 3′ spacer sequence are a combination of 5′61A8CA and 3′71A8CA.

7. The recombinant nucleic acid molecule for preparing the circular RNA according to claim 1, wherein the translation initiation element is an IRES sequence;
the IRES sequence is derived from one of Taura syndrome virus, Triatoma virus, Theiler's encephalomyelitis virus, simian virus 40, Solenopsis invicta virus 1, Rhopalosiphum padi virus, reticuloendotheliosis virus, Poliovirus type 1 (Mahoney), Chrysodeixis includens virus, Kashmir bee virus, human rhinovirus 2, Homalodisca coagulata virus-1, human immunodeficiency virus type 1, Homalodisca coagulata virus-1, Himetobi P virus, hepatitis C virus, hepatitis A virus, GB hepatitis virus, foot-and-mouth disease virus, human enterovirus 71, equine rhinitis virus, Ectropis obliqua picorna-like virus, encephalomyocarditis virus (EMCV), Drosophila C virus, crucifer-infecting tobamovirus, cricket paralysis virus, bovine viral diarrhea virus 1, black queen cell virus, aphid lethal paralysis virus, avian encephalomyelitis virus, acute bee paralysis virus, hibiscus chlorotic ringspot virus, classical swine fever virus, human FGF2, human SFTPA1, human AMLI/RUNXI, Drosophila antennapedia, human AQP4, human AT1R, human BAG-1, human BCL2, human BiP, human c-IAPI, human c-myc, human eIF4G, mouse NDST4L, human LEF1, mouse HIFia, human n-myc, mouse Gtx, human p27kip1, human PDGF2/c-sis, human p53, human Pim-1, mouse Rbm3, Drosophila reaper, canine Scamper, Drosophila Ubx, human UNR, mouse UtrA, human VEGF-A, human XIAP, salivirus, coxsackievirus, parechovirus, Drosophila hairless, Saccharomyces cerevisiae TFIID, Saccharomyces cerevisiae YAP1, human c-src, human FGF-1, simian picornavirus, turnip crinkle virus, aptamer of eIF4G, coxsackievirus B3 (CVB3), or coxsackievirus A (CVB1/2);
preferably, the IRES sequence is an IRES sequence of Coxsackievirus B3 (CVB3); more preferably, the IRES sequence is an IRES sequence of Encephalomyocarditis virus (EMCV).

8. The recombinant nucleic acid molecule for preparing the circular RNA according to claim 1, wherein the translation initiation element is a CVB3 IRES sequence into which an IRES enhancer element is inserted;
wherein the IRES enhancer element is inserted between domain I and domain II of the IRES sequence (designated as IRES-1), at a stem-loop structure of the domain II of the IRES sequence (designated as IRES-2), at a distal loop of domain IV of the IRES sequence (designated as IRES-3), or at a proximal loop of domain IV of the IRES sequence (designated as IRES-4);
the IRES enhancer element is a sequence set forth below:

| Serial No. | IRES sequence |
|---|---|
| A1 | CCGGCGGGT |
| A2 | GTTTCATTTTATTCC |
| | TATAC (SEQ ID NO: 46) |
| A3 | CAATTGAGAGATCGTTAC |
| | CATATAGCTA (SEQ ID NO: 47) |
| A4 | ATTTTATTCCTA (SEQ ID NO: 48) |

or repeat sequences of the above sequences:

| Serial | IRES repeat sequence |
|---|---|
| A1-X2 | CCGGCGGGTCCGGCGGG |
| | T (SEQ ID NO: 4950) |
| A1-X5 | CCGGCGGGTCCGGCGGGTCCG |
| | GCGGGTCCGGCGGGTCCGGC |
| | GGGT (SEQ ID NO: 501) |
| A2-X2 | GTTTCATTTTATTCCTATACG |
| | TTTCATTTTATTCCTATAC |
| | (SEQ ID NO: 512) |

9. The recombinant nucleic acid molecule for preparing the circular RNA according to claim 1, wherein the translation initiation element comprises at least one of an IRES sequence, a 5′ UTR sequence, a Kozak sequence, a sequence containing an m6A modification, or a complementary sequence of ribosomal 18S rRNA.

10. The recombinant nucleic acid molecule for preparing the circular RNA according to claim 1, wherein a target polypeptide coding region or a non-coding region is a protein coding region encoding a human protein or a non-human protein; wherein
the protein coding region encodes an antibody;
the human protein or the non-human protein is selected from a group consisting of hFIX, SP-B, VEGF-A, human methylmalonyl-CoA mutase (hMUT), CFTR, a cancer autoantigen, and a gene editing enzyme, wherein the gene editing enzyme includes Cpf1, a zinc finger nuclease (ZFN), and a transcription activator-like effector nuclease (TALEN);
a protein is a protein for therapeutic use;
the antibody is a human anti-HIV antibody;
the antibody is a bispecific antibody;
the bispecific antibody targets CD3 and CLDN6, or targets CD19 and CD22;
the protein is a protein for diagnostic use;
the protein coding region encodes Gaussia luciferase (Gluc), firefly luciferase (Fluc), enhanced green fluorescent protein (eGFP), human erythropoietin (hEPO), or Cas9 endonuclease;
wherein the protein coding region includes at least two coding regions, and any two adjacent coding regions are connected by a linker; and the linker is a polynucleotide encoding a 2A peptide; and
a translation initiation element is linked between any two adjacent coding regions; the translation initiation element located between any two adjacent coding regions comprises at least one of the following sequences: an IRES sequence, a 5′ UTR sequence, a Kozak sequence, a sequence comprising an m6A modification, or a complementary sequence of ribosome18S rRNA.

11. The recombinant nucleic acid molecule for preparing the circular RNA according to claim 1, wherein the recombinant nucleic acid molecule further comprises an insertion element located upstream of a translation initiation element; the insertion element is selected from at least one of a transcriptional regulatory element, a translational regulatory element, or a purification element;
wherein the insertion element comprises at least one of an untranslated region sequence, a polyA sequence, an aptamer sequence, a riboswitch sequence, or a sequence that binds a transcriptional regulatory factor.

12. The recombinant nucleic acid molecule for preparing the circular RNA according to claim 1, wherein a target polypeptide coding region or a non-coding region is a protein coding region encoding a human protein or a non-human protein; the recombinant nucleic acid molecule comprises homology arms, wherein each of the homology arms has a length of about 5-50 nucleotides, wherein each of the homology arms has a length of about 9-19 nucleotides.

13. A recombinant expression vector, wherein the recombinant expression vector comprises the recombinant nucleic acid molecule according to claim 1.

14. A circular RNA molecule, which is obtained by transcribing the recombinant nucleic acid molecule according to claim 1.

15. A composition, comprising the recombinant nucleic acid molecule according to claim 1, a recombinant expression vector comprising the recombinant nucleic acid molecule, or a circular RNA prepared based on the recombinant nucleic acid molecule; and
one or more pharmaceutically acceptable carriers;
wherein the pharmaceutically acceptable carrier comprises at least one of a lipid, a polymer, or a lipid-polymer complex.

16. An IRES enhancer element for insertion into a CVB3 IRES sequence to enhance a translation efficiency, wherein the IRES enhancer element is inserted between domain I and domain II of an IRES sequence (designated IRES-1), at a stem-loop structure of domain II of the IRES sequence (designated IRES-2), at a distal loop of domain IV of the IRES sequence (designated IRES-3), at a proximal loop of domain IV of the IRES sequence (designated IRES-4);
the IRES enhancer element is a sequence set forth below:

| Serial No. | IRES sequence |
|---|---|
| A1 | CCGGCGGGT |
| A2 | GTTTCATTTTATTCCT |
| | ATAC (SEQ ID NO: 46) |
| A3 | CAATTGAGAGATCGTTAC |
| | CATATAGCTA (SEQ ID |
| A4 | ATTTTATTCCT |
| | A (SEQ ID NO: 48) |

or repeat sequences of the above sequence:

| Serial | IRES repeat sequence |
|---|---|
| A1-X2 | CCGGCGGGTCCGGCGGGT |
| | (SEQ ID NO: 4950) |
| A1-X5 | CCGGCGGGTCCGGCGGGTCCGG |
| | CGGGTCCGGCGGGTCCGGCGG |
| | GT (SEQ ID NO: 501) |
| A2-X2 | GTTTCATTTTATTCCTATACGT |
| | TTCATTTTATTCCTATAC (SEQ ID |
| | NO: 512) |
